(19) United States
(12) Patent Application Publication
(10) Pub. No.: US 2024/0109910 A1
(43) Pub. Date: Apr. 4, 2024

(54) HEXAHYDRO-5,8-EPOXYCYCLOHEPTA[c]PYRAZOLE DERIVATIVES USEFUL AS MODULATORS OF THE CB1 AND / OR CB2 RECEPTORS

(71) Applicant: JANSSEN PHARMACEUTICA NV, BEERSE (BE)

(72) Inventors: SANATH K MEEGALLA, Gamet Valley, PA (US); Mark R. Player, Phoenixville, PA (US)

(73) Assignee: JANSSEN PHARMACEUTICA NV, BEERSE (BE)

(21) Appl. No.: 17/767,500

(22) PCT Filed: Oct. 9, 2020
(86) PCT No.: PCT/EP2020/078428

§ 371 Date: Apr. 8, 2022

Related U.S. Application Data
(60) Provisional application No. 62/912,842, filed on Oct. 9, 2019.

(51) Int. Cl. C07D 491/18

(52) U.S. Cl. CPC C07D 491/18

(57) ABSTRACT

The present invention is related to compounds of formula (I) and compounds of formula (II) and pharmaceutical composition thereof, useful as agonists of the CB1 and/or CB2 receptor(s).

### CROSS REFERENCE TO RELATED APPLICATION

This application claims priority to U.S. Provisional Application No. 62/912,842, filed Oct. 9, 2019. The disclosure is incorporated herein by reference in its entirety.

### FIELD OF THE INVENTION

The present invention is related to compounds of formula (I) and compounds of formula (II) and pharmaceutical composition thereof, useful as agonists of the CB1 and/or CB2 receptor(s), for the treatment of diseases, syndromes, conditions and/or disorders mediated by CB1 and/or CB2 receptor(s).

### BACKGROUND OF THE INVENTION

Cannabinoids are a group of extracellular signaling molecules found in both plants and animals. These molecules signal through two G-protein coupled receptors, Cannabinoid Receptor 1 (CB1) and Cannabinoid Receptor 2 (CB2). CB1 is mainly expressed in CNS neurons but also present in other peripheral tissues at lower concentrations (Matsuda, L. A. et al (1990) Nature 346: 561-564). The CB2 receptor is predominantly expressed in non-neuronal tissues, e.g. in hematopoietic cells, endothelial cells osteoblasts, osteoclasts, the endocrine pancreases, and cancerous cell lines (Packer, P. et al (2006) Pharmacol. Rev. 58 (3); 389-462). Consequently, CB1 is believed to be responsible for the psychotropic effects of cannabinoids and CB2 is responsible for their non-neural effects.

Compounds capable of CB receptor activity can be used in the treatment of CB receptor mediated syndromes, diseases or disorders which include appetite, metabolism, diabetes, obesity, glaucoma associated intra-ocular pressure, mood disorders, seizures, substance abuse, leaning disorders, cognition disorders, memory disorders, organ contraction, muscle spasm, respiratory disorders, locomotor activity disorders, movement disorders, immune disorders, immune disorders, inflammation, cell growth disorders, eye-disease, allergies and allergic reactions, pain, anxiety, psychotic afflictions, pathological states of brain, gastrointestinal disorders, nausea, vomiting, giddiness, urinary and fertility problems, cardiovascular disease, neuroinflammatory pathologies, dermatological disorders and nephrological disorders.

There remains a need for CB1, CB2 and dual CB1/CB2 agonists for the treatment of syndromes, diseases or disorders which include appetite, metabolism, diabetes, obesity, glaucoma associated intra-ocular pressure, mood disorders, seizures, substance abuse, leaning disorders, cognition disorders, memory disorders, organ contraction, muscle spasm, respiratory disorders, locomotor activity disorders, movement disorders, immune disorders, immune disorders, inflammation, cell growth disorders, eye-disease, allergies and allergic reactions, pain, anxiety, psychotic afflictions, pathological states of brain, gastrointestinal disorders, nausea, vomiting, giddiness, urinary and fertility problems, cardiovascular disease, neuroinflammatory pathologies, dermatological disorders and nephrological disorders.

### SUMMARY OF THE INVENTION

Provided herein are compounds useful as CB1, CB2 and dual CB1/CB2 agonists. In one aspect, provided herein are compounds, and pharmaceutically acceptable salts thereof, having formula (I) and formula (II)

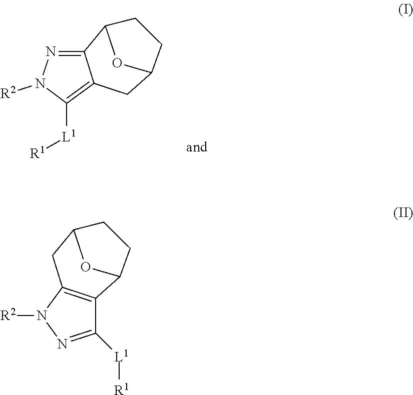

wherein
L¹-R¹ is selected from the group consisting of
—C(O)—NH—R¹,

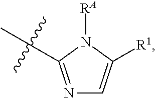

wherein R^{A} is hydrogen, methyl or ethyl;

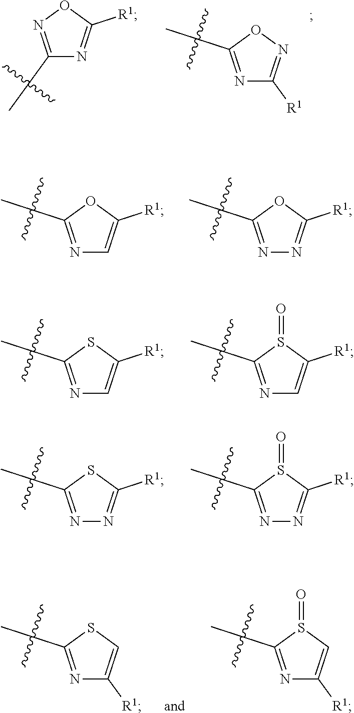

R¹ is selected from the group consisting of C₁₋₁₂alkyl, C₃₋₁₂cycloalkyl and -(C₁₋₂alkyl)-C₃₋₁₂cycloalkyl;
wherein the C₁₋₁₂alkyl or C₃₋₁₂cycloalkyl, whether alone or as part of a substituent group may be optionally substituted with one to three substituents independently selected from the group consisting of halogen, hydroxy and fluorinated C₁₋₄alkyl;
provided that when -L¹-R¹ is other than —C(O)—NH—R¹, then R¹ is selected from the group consisting of C₁₋₁₂alkyl; wherein the C₁₋₁₂alkyl is optionally substituted with one to three substituents independently selected from the group consisting of halogen, hydroxy and fluorinated C₁₋₄alkyl,
R² is selected from the group consisting of phenyl, pyrazin-2-yl and pyrazin-2-yl -1-oxide;
wherein the phenyl is optionally substituted with one or more substituents independently selected from the group consisting of halogen, C₁₋₂alkyl and CF₃;
provided that when -L¹-R¹ is selected from the group consisting of

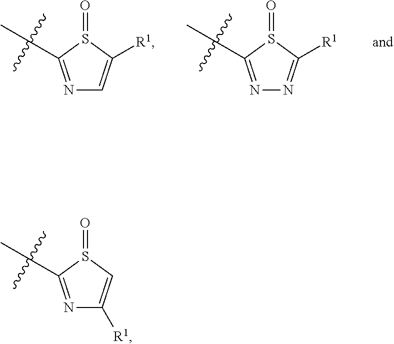

then R² is pyrazin-2-yl-1-oxide.

The present invention also provides a pharmaceutical composition comprising a therapeutically effective amount of a compound for formula (I) and/or formula (II), or a pharmaceutically acceptable salt thereof for use in medicine, and optionally a pharmaceutically acceptable carrier. The pharmaceutical composition may be used in human or veterinary medicine.

The present invention further provides a method of treating syndromes, disorders, conditions and/or diseases associated with CB1 and/or CB2 receptors, the method comprising administering to a patient in need thereof a therapeutically effective amount of a compound of or composition comprising a compound of formula (I) and/or formula (II), or a pharmaceutically acceptable salt thereof. Syndromes, disorders, conditions and/or diseases associated with CB1 and/or CB2 receptors include, but are not limited to, appetite, metabolism, diabetes, obesity, glaucoma associated intra-ocular pressure, mood disorders, seizures, substance abuse, leaning disorders, cognition disorders, memory disorders, organ contraction, muscle spasm, respiratory disorders, locomotor activity disorders, movement disorders, immune disorders, immune disorders, inflammation, cell growth disorders, eye-disease, allergies and allergic reactions, pain, anxiety, psychotic afflictions, pathological states of brain, gastrointestinal disorders, nausea, vomiting, giddiness, urinary and fertility problems, cardiovascular disease, neuroinflammatory pathologies, dermatological disorders and nephrological disorders, neurodegenerative disorders, eating disorders, fibrotic diseases, or for weight loss, weight control or treatment of obesity.

The present invention also provides a compound for use in any of the methods described herein. The present invention further provides use of a compound for the preparation of a medicament for use in any of the methods described herein. The details of one or more embodiments of the invention are set forth in the description below. Other features, objects, and advantages of the invention will be apparent from the description and claims.

### DETAILED DESCRIPTION OF THE INVENTION

The present invention is directed to compounds of formula (I) and compounds of formula (II), useful as agonists of the CB1 and/or CB2 receptors. The compound of formula (I) and formula (II) are useful for the treatment of syndromes, diseases or disorders which include appetite, metabolism, diabetes, obesity, glaucoma associated intra-ocular pressure, mood disorders, seizures, substance abuse, leaning disorders, cognition disorders, memory disorders, organ contraction, muscle spasm, respiratory disorders, locomotor activity disorders, movement disorders, immune disorders, immune disorders, inflammation, cell growth disorders, eye-disease, allergies and allergic reactions, pain, anxiety, psychotic afflictions, pathological states of brain, gastrointestinal disorders, nausea, vomiting, giddiness, urinary and fertility problems, cardiovascular disease, neuroinflammatory pathologies, dermatological disorders and nephrological disorders, pain, neurodegenerative disorders, eating disorders, fibrotic diseases, or for weight loss, weight control or the treatment of obesity.

In an embodiment of the present invention, the compounds of formula (I) and/or the compounds of formula (II) are agonists of the CBI receptor. In another embodiment of the present invention, the compounds of formula (I) and/or the compounds of formula (II) are agonists of the CB2 receptor. In another embodiment of the present invention, the compounds of formula (I) and/or the compounds of formula (II) are dual agonists of the CB1 receptor and the CB2 receptor.

Also, in other embodiments of the present invention compounds and compositions of formula (I) and/or the compounds and compositions of formula (II) are used in therapeutically effective amounts to treat subjects having syndromes, diseases or disorders including, but not limited to, appetite, metabolism, diabetes, obesity, glaucoma associated intra-ocular pressure, mood disorders, seizures, substance abuse, leaning disorders, cognition disorders, memory disorders, organ contraction, muscle spasm, respiratory disorders, locomotor activity disorders, movement disorders, immune disorders, immune disorders, inflammation, cell growth disorders, eye-disease, allergies and allergic reactions, pain, anxiety, psychotic afflictions, pathological states of brain, gastrointestinal disorders, nausea, vomiting, giddiness, urinary and fertility problems, cardiovascular disease, neuroinflammatory pathologies, dermatological disorders and nephrological disorders, pain, neurodegenerative disorders, eating disorders, fibrotic diseases, or for weight loss, weight control or the treatment of obesity. Other embodiments include the use of compounds and compositions of formula (I) and/or formula (II) in making pharmaceutical compositions for the treatment of these diseases, disorders, syndromes and conditions.

### Exemplary Compounds and Compositions

Provided herein, inter alia, are compounds of formula (I) and compounds of formula (II)

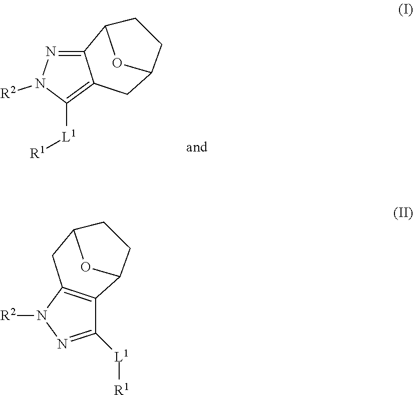

wherein R¹, R² and L¹ are as herein defined, and pharmaceutically acceptable salts thereof.

One skilled in the art will recognize that throughout the specification and claims, “L¹-R¹” is intended to denote the combination of a linking ring L¹ substituted with an R¹ group; and to specifically define the binding of the L¹ and R¹ substituents relative to each other and the rest of the compound of formula (I) to which they are bound. For example, wherein L¹-R¹ is

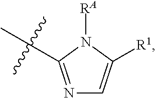

then the L¹ group is the

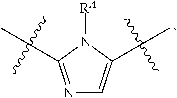

wherein the L¹ group is bound to the rest of the compound of formula (I) through the 2-position of the R^{A}-substituted imidazole (L¹ group), and wherein the R¹ group is bound to the 5-position of the R^{A}-substituted imidazole (L¹ group).

In certain embodiments, the present invention is directed to compounds of formula (I) and/or compounds of formula (II) wherein L¹-R¹ is —C(O)—NH—R¹.

In certain embodiments, the present invention is directed to compounds of formula (I) and/or compounds of formula (II) wherein L¹-R¹ is

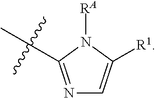

In certain embodiments, the present invention is directed to compounds of formula (I) and/or compounds of formula (II) wherein L¹-R¹ is selected from the group consisting of

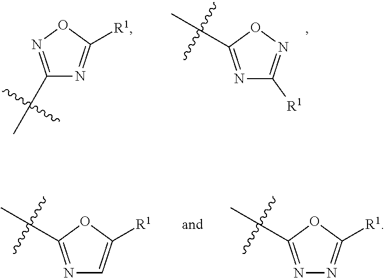

In certain embodiments, the present invention is directed to compounds of formula (I) and/or compounds of formula (II) wherein L¹-R¹ is selected from the group consisting of

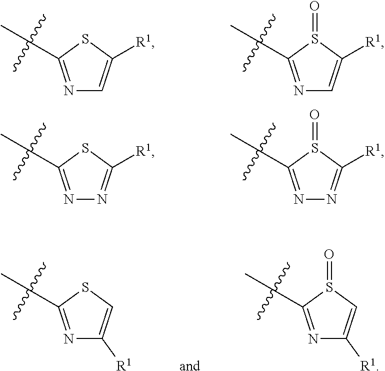

In certain embodiments, the present invention is directed to compounds of formula (I) and/or compounds of formula (II) wherein L¹-R¹ is selected from the group consisting of —C(O)—NH—R¹,

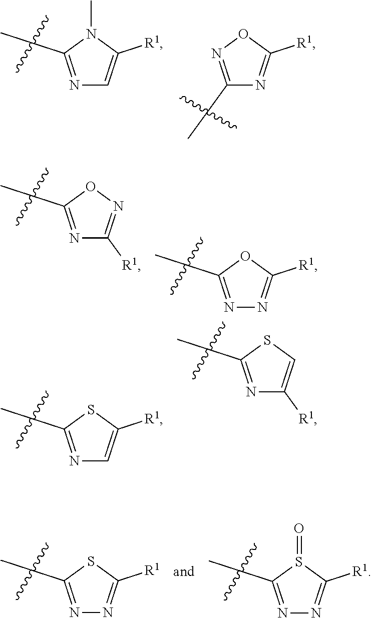

In certain embodiments, the present invention is directed to compounds of formula (I) and/or compounds of formula (II) wherein R^{A} is selected from the group consisting of hydrogen, methyl and ethyl. In certain embodiments, the present invention is directed to compounds of formula (I) and or compounds of formula (II) wherein R^{A} is selected from the group consisting of hydrogen and methyl. In certain embodiments, the present invention is directed to compounds of formula (I) and/or compounds of formula (II) wherein R^{A} is selected from the group consisting of hydrogen and methyl.

In certain embodiments, the present invention is directed to compounds of formula (I) and/or compounds of formula (II) wherein R¹ is selected from the group consisting of C₁₋₈alkyl, C₃₋₁₂cycloalkyl and -(C₁₋₂alkyl)-C₃₋₁₂cycloalkyl; wherein the C₁₋₈alkyl or C₃₋₁₂cycloalkyl, whether alone or as part of a substituent group may be optionally substituted with one to two substituents independently selected from the group consisting of halogen, hydroxy and fluorinated C₁₋₂alkyl.

In certain embodiments, the present invention is directed to compounds of formula (I) and/or compounds of formula (II) wherein R¹ is selected from the group consisting of C₁₋₆alkyl, C₃₋₁₂cycloalkyl and —(CH₂)-C₃₋₁₂cycloalkyl; wherein the C₁₋₆alkyl or C₃₋₁₂cycloalkyl, whether alone or as part of a substituent group may be optionally substituted a substituent selected from the group consisting of hydroxy and fluorinated C₁₋₂alkyl.

In certain embodiments, the present invention is directed to compounds of formula (I) and/or compounds of formula (II) wherein R¹ is selected from the group consisting of t-butyl, 2-methyl-n-but-2-yl, 1,1-dimethyl-2-hydroxy-ethyl, 1-hydroxy-3,3-dimethyl-n-but-2-yl, 1-(trifluoromethyl)-cycloprop-1-yl, 1-(trifluoromethyl)-cyclobut-1-yl, bicyclo[1.1.1]pent-1-yl, bicyclo[2.2.2]octan-1-yl, octahydro-2,5-methanopentalen-3-yl and (1R,3R,5S,7R)-tetracyclo[5.2.1.0^{3,8}.0^{5,8}]decan-3-yl-methyl and adamant-1-yl -methyl-.

In certain embodiments, the present invention is directed to compounds of formula (I) and/or compounds of formula (II) wherein R¹ is selected from the group consisting of t-butyl, octahydro-2,5-methanopentalen-3-yl and adamant-1-yl-methyl-.

In certain embodiments, the present invention is directed to compounds of formula (I) and/or compounds of formula (II) wherein R¹ is selected from the group consisting of t-butyl, 2-methyl-n-but-2-yl, 1,1-dimethyl-2-hydroxy-ethyl, 1-hydroxy-3,3-dimethyl-n-but-2-yl, 1-(trifluoromethyl)-cycloprop-1-yl, 1-(trifluoromethyl)-cyclobut-1-yl, bicyclo[1.1.1]pent-1-yl, bicyclo[2.2.2]octan-1-yl, octahydro-2,5-methanopentalen-3-yl and (1R,3R,5S,7R)-tetracyclo[5.2.1.0^{3,8}.0^{5,8}]decan-3-yl-methyl.

In certain embodiments, the present invention is directed to compounds of formula (I) and/or compounds of formula (II) wherein R¹ is selected from the group consisting of t-butyl, 1,1-dimethyl-2-hydroxy-ethyl, 1-hydroxy-3,3-dimethyl-n-but-2-yl, 1-(trifluoro-methyl)-cycloprop-1-yl and 1-(trifluoro-methyl)-cyclobut-1-yl.

In certain embodiments, the present invention is directed to compounds of formula (I) and/or compounds of formula (II) wherein R² is selected from the group consisting of phenyl, pyrazin-2-yl and pyrazin-2-yl-1-oxide; wherein the phenyl is optionally substituted with one to two substituents independently selected from the group consisting of halogen, C₁₋₂alkyl and CF₃.

In certain embodiments, the present invention is directed to compounds of formula (I) and/or compounds of formula (II) wherein R² is selected from the group consisting of phenyl, pyrazin-2-yl and pyrazin-2-yl-1-oxide; wherein the phenyl is optionally substituted with one to two substituents independently selected from the group consisting of fluoro and CF₃.

In certain embodiments, the present invention is directed to compounds of formula (I) and/or compounds of formula (II) wherein R² is selected from the group consisting of 2,4-difluorophenyl, pyrazin-2-yl and pyrazin-3-yl-1-oxide. In certain embodiments, the present invention is directed to compounds of formula (I) and/or compounds of formula (II) wherein R² is selected from the group consisting of pyrazin-2-yl and pyrazin-3-yl-1-oxide.

Certain embodiments of the present invention include those wherein the substituents selected for one or more of the variables defined herein (i.e. L¹, R¹, R², etc.) are independently selected to be any individual substituent or any subset of substituents selected from the complete list as defined herein. Additional embodiments of the present invention, include those wherein the substituents selected for one or more of the variables defined herein (i.e. L¹, R¹, R², etc.) are independently selected to correspond to any of the embodiments as defined herein.

In another embodiment of the present invention is any single compound or subset of compounds selected from the representative compounds listed in Table 1 and/or Table 2, below.

Specific, non-limiting compounds of the present invention are found in Table 1 and Table 2 below:

TABLE 1
Representative Compounds of Formula (1)

| Compound # | Structure | -L¹-R¹ | R¹ | R² |
|---|---|---|---|---|
| 2 | 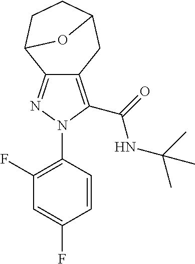 [SMILES: CC(C)(C)NC(=O)c1c2c(nn1-c1ccc(F)cc1F)C1CCC(C2)O1] | —C(O)—NH—R¹ | t-butyl | 2,4-difluoro- phenyl |
| 4 | 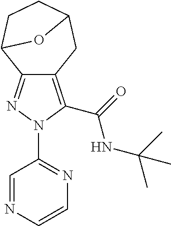 [SMILES: CC(C)(C)NC(=O)c1c2c(nn1-c1cnccn1)C1CCC(C2)O1] | —C(O)—NH—R¹ | t-butyl | pyrazin-2-yl |
| 5 | 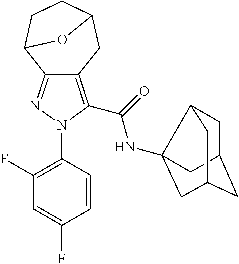 [SMILES: O=C(NC12CC3CC(CC1C3)C2)c1c2c(nn1-c1ccc(F)cc1F)C1CCC(C2)O1] | —C(O)—NH—R¹ | octahydro-2,5- methano pentalen-3-yl | 2,4-difluoro- phenyl |
| 6 | 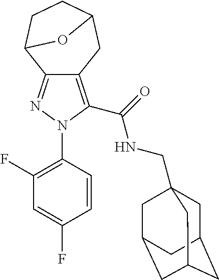 [SMILES: O=C(NCC12CC3CC(CC(C3)C1)C2)c1c2c(nn1-c1ccc(F)cc1F)C1CCC(C2)O1] | —C(O)—NH—R¹ | adamant-1-yl- methyl- | 2,4-difluoro- phenyl |
| 30 | 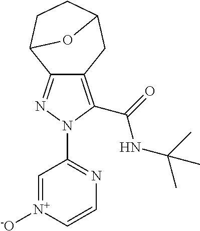 | —C(O)—NH—R¹ | t-butyl | pyrazin-3-yl- 1-oxide |
| 46 | 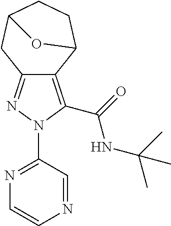 [SMILES: CC(C)(C)NC(=O)c1c2c(nn1-c1cnccn1)CC1CCC2O1] | —C(O)—NH—R¹ | t-butyl | pyrazin-2-yl |
| 49 | 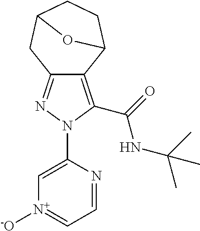 | —C(O)—NH—R¹ | t-butyl | pyrazin-3-yl- 1-oxide |

TABLE 2
Representative Compounds of Formula (I)

| Compound # | Structure | -L¹-R¹ | R¹ | R² |
|---|---|---|---|---|
| 1 | 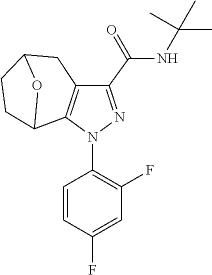 [SMILES: CC(C)(C)NC(=O)c1nn(-c2ccc(F)cc2F)c2c1CC1CCC2O1] | —C(O)—NH—R¹ | t-butyl | 2,4-difluoro- phenyl |
| 3 | 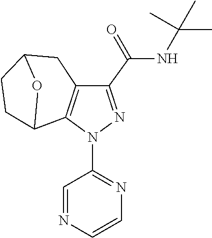 [SMILES: CC(C)(C)NC(=O)c1nn(-c2cnccn2)c2c1CC1CCC2O1] | —C(O)—NH—R¹ | t-butyl | pyrazin-2-yl |
| 7 | 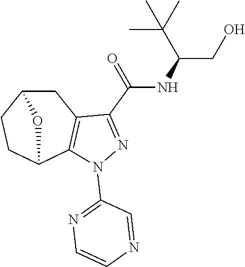 [SMILES: CC(C)(C)[C@@H](CO)NC(=O)c1nn(-c2cnccn2)c2c1C[C@@H]1CC[C@H]2O1] | —C(O)—NH—R¹ | 1-hydroxy-3,3- dimethyl-n-but- 2-yl | pyrazin-2-yl |
| 8 | 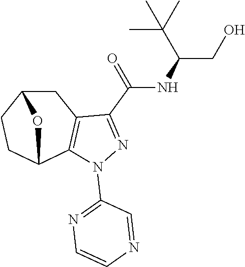 [SMILES: CC(C)(C)[C@@H](CO)NC(=O)c1nn(-c2cnccn2)c2c1C[C@H]1CC[C@@H]2O1] | —C(O)—NH—R¹ | 1-hydroxy-3,3- dimethyl-n-but- 2-yl | pyrazin-2-yl |
| 9 | 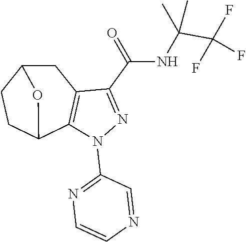 [SMILES: CC(C)(NC(=O)c1nn(-c2cnccn2)c2c1CC1CCC2O1)C(F)(F)F] | —C(O)—NH—R¹ | 1-(trifluoro- methyl)- cycloprop-1-yl | pyrazin-2-yl |
| 10 | 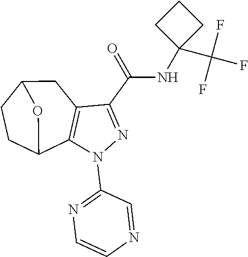 [SMILES: O=C(NC1(C(F)(F)F)CCC1)c1nn(-c2cnccn2)c2c1CC1CCC2O1] | —C(O)—NH—R¹ | 1-(trifluoro- methyl)- cyclobut-1-yl | pyrazin-2-yl |
| 11 | 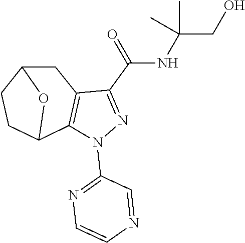 [SMILES: CC(C)(CO)NC(=O)c1nn(-c2cnccn2)c2c1CC1CCC2O1] | —C(O)—NH—R¹ | 1,1-dimethyl-2- hydroxy-ethyl | pyrazin-2-yl |
| 12 | 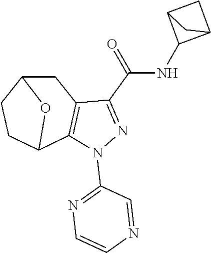 [SMILES: O=C(NC1C2CC1C2)c1nn(-c2cnccn2)c2c1CC1CCC2O1] | —C(O)—NH—R¹ | bicyclo[1.1.1] pent-1-yl | pyrazin-2-yl |
| 13 | 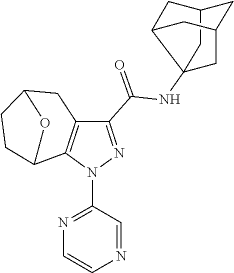 [SMILES: O=C(NC12CC3CC(CC1C3)C2)c1nn(-c2cnccn2)c2c1CC1CCC2O1] | —C(O)—NH—R¹ | octahydro-2,5- methano pentalen-3-yl | pyrazin-2-yl |
| 14 | 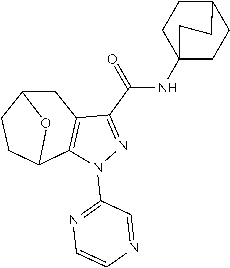 [SMILES: O=C(NC12CCC(CC1)CC2)c1nn(-c2cnccn2)c2c1CC1CCC2O1] | —C(O)—NH—R¹ | bicyclo[2.2.2] octan-1-yl | pyrazin-2-yl |
| 15 | 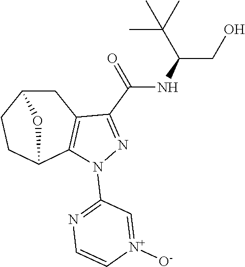 [SMILES: CC(C)(C)[C@@H](CO)NC(=O)c1nn(-c2c[n+]([O-])ccn2)c2c1C[C@@H]1CC[C@H]2O1] | —C(O)—NH—R¹ | 1-hydroxy-3,3- dimethyl-n-but- 2-yl | pyrazin-3-yl- 1-oxide |
| 16 | 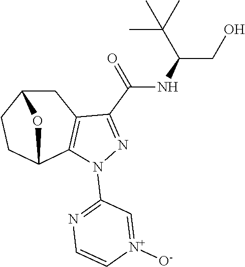 [SMILES: CC(C)(C)[C@@H](CO)NC(=O)c1nn(-c2c[n+]([O-])ccn2)c2c1C[C@H]1CC[C@@H]2O1] | —C(O)—NH—R¹ | 1-hydroxy-3,3- dimethyl-n-but- 2-yl | pyrazin-3-yl- 1-oxide |
| 17 | 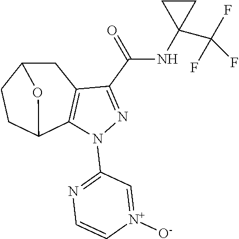 [SMILES: O=C(NC1(C(F)(F)F)CC1)c1nn(-c2c[n+]([O-])ccn2)c2c1CC1CCC2O1] | —C(O)—NH—R¹ | 1-(trifluoro- methyl)- cycloprop-1-yl | pyrazin-3-yl- 1-oxide |
| 18 | 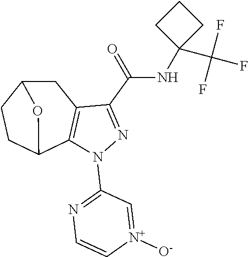 [SMILES: O=C(NC1(C(F)(F)F)CCC1)c1nn(-c2c[n+]([O-])ccn2)c2c1CC1CCC2O1] | —C(O)—NH—R¹ | 1-(trifluoro- methyl)- cyclobut-1-yl | pyrazin-3-yl- 1-oxide |
| 19 | 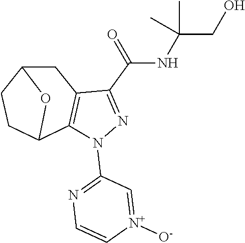 [SMILES: CC(C)(CO)NC(=O)c1nn(-c2c[n+]([O-])ccn2)c2c1CC1CCC2O1] | —C(O)—NH—R¹ | 1,1-dimethyl-2- hydroxy-ethyl | pyrazin-3-yl- 1-oxide |
| 20 | 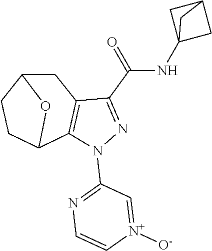 [SMILES: O=C(NC12CC(C1)C2)c1nn(-c2c[n+]([O-])ccn2)c2c1CC1CCC2O1] | —C(O)—NH—R¹ | bicyclo[1.1.1] pent-1-yl | pyrazin-3-yl- 1-oxide |
| 21 | 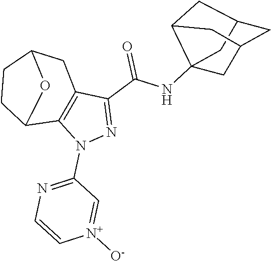 [SMILES: O=C(NC12CC3CC(CC1C3)C2)c1nn(-c2c[n+]([O-])ccn2)c2c1CC1CCC2O1] | —C(O)—NH—R¹ | octahydro-2,5- methano pentalen-3-yl | pyrazin-3-yl- 1-oxide |
| 22 | 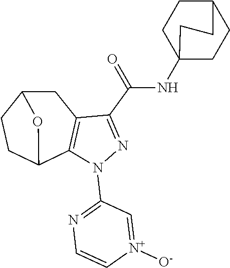 [SMILES: O=C(NC12CCC(CC1)CC2)c1nn(-c2c[n+]([O-])ccn2)c2c1CC1CCC2O1] | —C(O)—NH—R¹ | bicyclo[2.2.2] octan-1-yl | pyrazin-3-yl- 1-oxide |
| 23 | 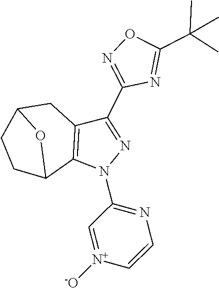 | 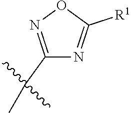 | t-butyl | pyrazin-3-yl- 1-oxide |
| 24 | 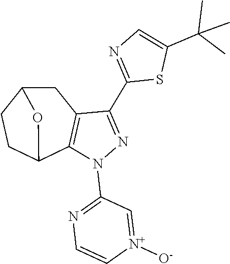 [SMILES: CC(C)(C)c1cnc(-c2nn(-c3c[n+]([O-])ccn3)c3c2CC2CCC3O2)s1] | 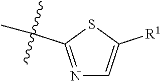 | t-butyl | pyrazin-3-yl- 1-oxide |
| 25 | 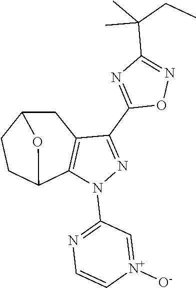 [SMILES: CCC(C)(C)c1noc(-c2nn(-c3c[n+]([O-])ccn3)c3c2CC2CCC3O2)n1] | 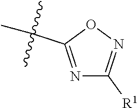 | 2-methyl-n-but- 2-yl | pyrazin-3-yl- 1-oxide |
| 26 | 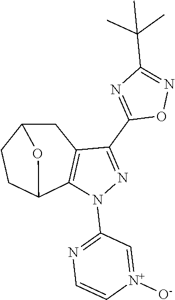 [SMILES: CC(C)(C)c1noc(-c2nn(-c3c[n+]([O-])ccn3)c3c2CC2CCC3O2)n1] | 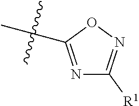 | t-butyl | pyrazin-3-yl- 1-oxide |
| 27 | 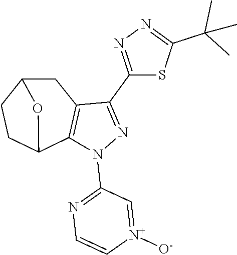 [SMILES: CC(C)(C)c1nnc(-c2nn(-c3c[n+]([O-])ccn3)c3c2CC2CCC3O2)s1] | 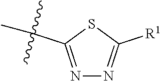 | t-butyl | pyrazin-3-yl- 1-oxide |
| 28 | 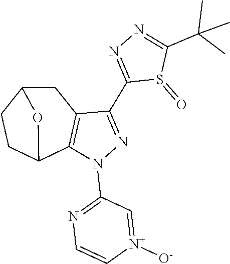 [SMILES: CC(C)(C)C1=NN=C(c2nn(-c3c[n+]([O-])ccn3)c3c2CC2CCC3O2)S1=O] | 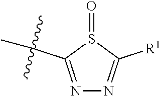 | t-butyl | pyrazin-3-yl- 1-oxide |
| 29 | 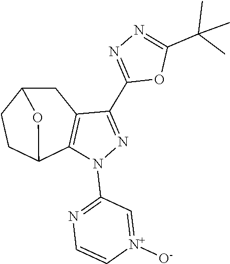 [SMILES: CC(C)(C)c1nnc(-c2nn(-c3c[n+]([O-])ccn3)c3c2CC2CCC3O2)o1] | 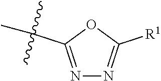 | t-butyl | pyrazin-3-yl- 1-oxide |
| 31 | 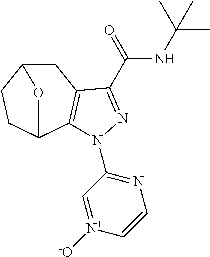 | —C(O)—NH—R¹ | t-butyl | pyrazin-3-yl- 1-oxide |
| 32 | 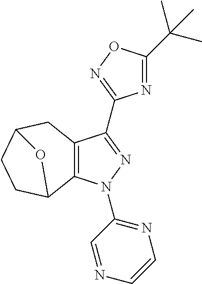 [SMILES: CC(C)(C)c1nc(-c2nn(-c3cnccn3)c3c2CC2CCC3O2)no1] | 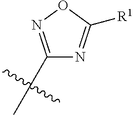 | t-butyl | pyrazin-2-yl |
| 33 | 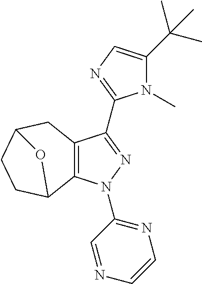 [SMILES: Cn1c(C(C)(C)C)cnc1-c1nn(-c2cnccn2)c2c1CC1CCC2O1] | 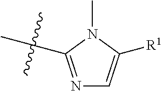 | t-butyl | pyrazin-2-yl |
| 34 | 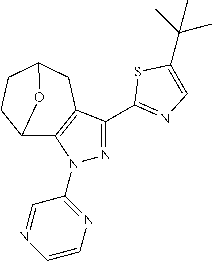 [SMILES: CC(C)(C)c1cnc(-c2nn(-c3cnccn3)c3c2CC2CCC3O2)s1] | 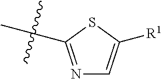 | t-butyl | pyrazin-2-yl |
| 35 | 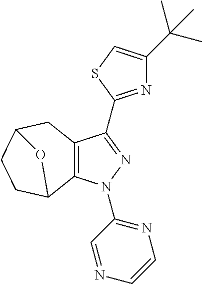 [SMILES: CC(C)(C)c1csc(-c2nn(-c3cnccn3)c3c2CC2CCC3O2)n1] | 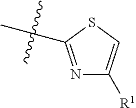 | t-butyl | pyrazin-2-yl |
| 36 | 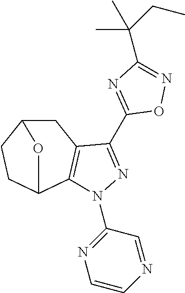 [SMILES: CCC(C)(C)c1noc(-c2nn(-c3cnccn3)c3c2CC2CCC3O2)n1] | 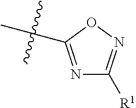 | 2-methyl-n-but- 2-yl | pyrazin-2-yl |
| 37 | 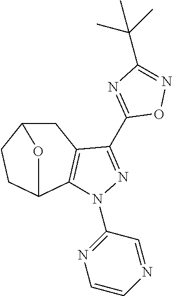 [SMILES: CC(C)(C)c1noc(-c2nn(-c3cnccn3)c3c2CC2CCC3O2)n1] | 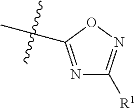 | t-butyl | pyrazin-2-yl |
| 38 | 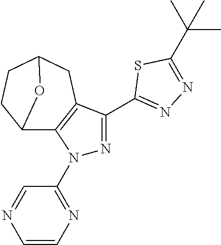 [SMILES: CC(C)(C)c1nnc(-c2nn(-c3cnccn3)c3c2CC2CCC3O2)s1] | 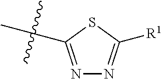 | t-butyl | pyrazin-2-yl |
| 39 | 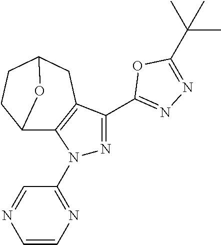 [SMILES: CC(C)(C)c1nnc(-c2nn(-c3cnccn3)c3c2CC2CCC3O2)o1] | 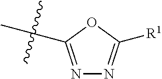 | t-butyl | pyrazin-2-yl |
| 40 | 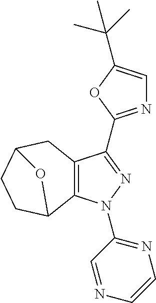 [SMILES: CC(C)(C)c1cnc(-c2nn(-c3cnccn3)c3c2CC2CCC3O2)o1] | 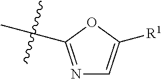 | t-butyl | pyrazin-2-yl |
| 41 | 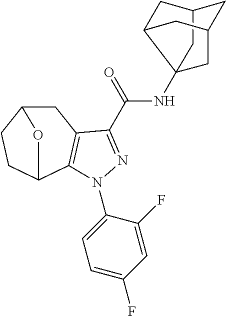 [SMILES: O=C(NC12CC3CC(CC1C3)C2)c1nn(-c2ccc(F)cc2F)c2c1CC1CCC2O1] | —C(O)—NH—R¹ | octahydro-2,5- methano pentalen-3-yl | 2,4-difluoro- phenyl |
| 42 | 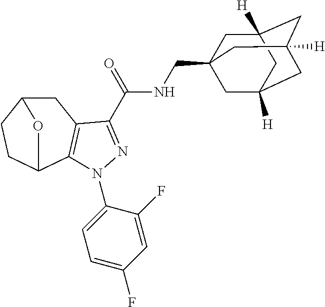 [SMILES: O=C(NC[C@]12C[C@@H]3C[C@@H](C[C@@H](C3)C1)C2)c1nn(-c2ccc(F)cc2F)c2c1CC1CCC2O1] | —C(O)—NH—R¹ | (1R,3R,5S,7R)- tetracyclo- [5.2.1.0^{3,8},0^{5,8}]- decan-3-yl- methyl | 2,4-difluoro- phenyl |
| 44 | 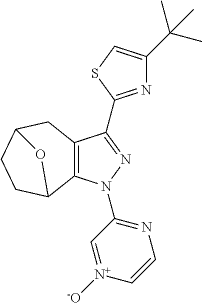 | 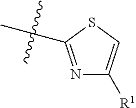 | t-butyl | pyrazin-3-yl- 1-oxide |
| 45 | 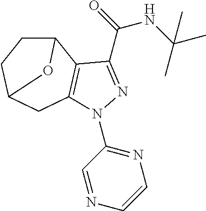 [SMILES: CC(C)(C)NC(=O)c1nn(-c2cnccn2)c2c1C1CCC(C2)O1] | —C(O)—NH—R¹ | t-butyl | pyrazin-2-yl |
| 47 | 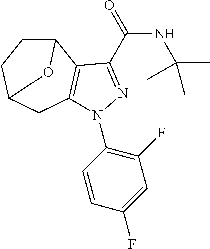 [SMILES: CC(C)(C)NC(=O)c1nn(-c2ccc(F)cc2F)c2c1C1CCC(C2)O1] | —C(O)—NH—R¹ | t-butyl | 2,4-difluoro- phenyl |
| 48 | 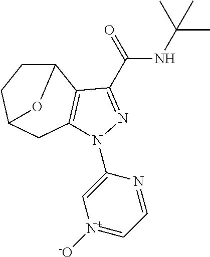 | —C(O)—NH—R¹ | t-butyl | pyrazin-3-yl-1- oxide |

In another embodiment, the present invention is directed to Compound #50, a compound of the following structure

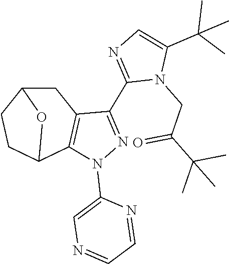 [SMILES: CC(C)(C)C(=O)Cn1c(C(C)(C)C)cnc1-c1nn(-c2cnccn2)c2c1CC1CCC2O1]

also known as 1-(5-(tert-butyl)-2-(1-(pyrazin-2-yl)-1,4,5,6,7,8-hexahydro-5,8-epoxycyclohepta[c]pyrazol-3-yl)-1H-imidazol-1-yl)-3,3-dimethylbutan-2-one, and pharmaceutically acceptable salts thereof.

In certain embodiments, the present invention is directed to one or more compounds independently selected from the group consisting of
N-(tert-butyl)-1-(pyrazin-2-yl)-1,4,5,6,7,8-hexahydro-5,8-epoxycyclohepta[c]pyrazole-3-carboxamide;
3-(3-(tert-butylcarbamoyl)-5,6,7,8-tetrahydro-5,8-epoxycyclohepta[c]pyrazol -1(4H)-yl)pyrazine 1-oxide;
N-(1-hydroxy-2-methylpropan-2-yl)-1-(pyrazin-2-yl)-1,4,5,6,7,8-hexahydro-5,8-epoxycyclohepta[c]pyrazole-3-carboxamide;
3-(3-((1-hydroxy-2-methylpropan-2-yl)carbamoyl)-5,6,7,8-tetrahydro-5,8-epoxycyclohepta[c]pyrazol-1(4H)-yl)pyrazine 1-oxide;
1-(pyrazin-2-yl)-N-(1-(trifluoromethyl)cyclopropyl)-1,4,5,6,7,8-hexahydro-5,8-epoxycyclohepta[c]pyrazole-3-carboxamide;
3-(5S,8R)-3-(((S)-1-hydroxy-3,3-dimethylbutan-2-yl)carbamoyl)-5,6,7,8-tetrahydro-5,8-epoxycyclohepta[c]pyrazol-1(4H)-yl)pyrazine 1-oxide;
34(5R,8S)-3-(((S)-1-hydroxy-3,3-dimethylbutan-2-yl)carbamoyl)-5,6,7,8-tetrahydro-5,8-epoxycyclohepta[c]pyrazol-1(4H)-yl)pyrazine 1-oxide;
1-(pyrazin-2-yl)-N-(1-(trifluoromethyl)cyclobutyl)-1,4,5,6,7,8-hexahydro-5,8-epoxycyclohepta[c]pyrazole-3-carboxamide;
3-(3-((1-(trifluoromethyl)cyclopropyl)carbamoyl)-5,6,7,8-tetrahydro-5,8-epoxycyclohepta[c]pyrazol-1(4H)-yl)pyrazine 1-oxide;
and pharmaceutically acceptable salts thereof.

In certain embodiments, the present invention is directed to compounds of formula (I) and/or compounds of formula (II) wherein the compound exhibits a measured EC₅₀ (agonism of the hCB2 receptor), measured as described in Biological Example 1 which follows hereinafter, of less than or equal to about 5 μM, preferably less than or equal to about 1 μM, more preferably less than or equal to about 500 nM, more preferably less than or equal to about 100 nM, more preferably less than or equal to about 50 nM, more preferably less than or equal to about 10 nM, more preferably less than or equal to about 1 nM, more preferably less than or equal to about 0.10 nM, more preferably less than or equal to about 0.05 nM

In certain embodiments, the present invention is directed to compounds of formula (I) and/or compounds of formula (II) wherein the compound exhibits a measured EC₅₀ (agonism of the hCB1 receptor), measured as described in Biological Example 1 which follows hereinafter, of less than or equal to about 5 μM, preferably less than or equal to about 1 μM, more preferably less than or equal to about 500 nM, more preferably less than or equal to about 100 nM, more preferably less than or equal to about 50 nM, more preferably less than or equal to about 20 nM, more preferably less than or equal to about 10 nM, more preferably less than or equal to about 1 nM.

The present invention also provides a pharmaceutical composition comprising a therapeutically effective amount of a compound of formula (I) and/or formula (II), or a pharmaceutically acceptable salt thereof for use in medicine, and optionally a pharmaceutically acceptable carrier. The pharmaceutical composition may be used in human and veterinary medicine.

### Certain Definitions

As used herein, “halogen” shall mean chloro, brava, fluoro and iodo, preferably chloro, bromo or fluoro, more preferably fluoro.

As used herein, the term “C_{X-Y}alkyl” wherein X and Y are integers, whether used alone or as part of a substituent group, include straight and branched chains containing between X and Y carbon atoms. For example, C₁₋₄alkyl radicals include straight and branched chains of between 1 and 4 carbon atoms, including methyl, ethyl, propyl, isopropyl, butyl, isobutyl, sec-butyl and t-butyl.

One skilled in the art will recognize that the term “-C_{X-Y}alkyl)-”, wherein X and Y are integers, shall denote any straight or branched C_{X-Y}alkyl carbon chain as herein defined, wherein said C_{X-Y}alkyl chain is divalent and is further bound through two points of attachment, preferably through two terminal carbon atoms. For example, the term “-(C₁₋₂alky)-” shall include —CH₂— and —CH₂CH₂—.

As used herein, unless otherwise noted, the term “fluorinated C_{X-Y}alkyl” shall mean any C_{X-Y}alkyl group as defined above, substituted with at least one fluorine atom. For example, C₁₋₄alkyl include, but are not limited to, —CF₃, —CH₂—CF₃, —CF₂—CF₂—CF₂—CF₃, and the like. Preferably, the fluorinated CX-Yalkyl is selected from the group consisting —CF₃, —CH₂CF₃ and —CF₂—CF₃, more preferably —CF₃.

As used herein, unless otherwise noted, the term “C_{X-Y}cycloalkyl”, wherein X and Y are integers, shall mean any stable X- to Y-membered monocyclic, bicyclic, spiro-cyclic and/or bridged saturated ring system. For example, the term “C₃₋₆cycloalkyl” shall include cyclopropyl, cyclobutyl, cyclopentyl and cyclohexyl.

In certain embodiments of the present invention, the C₃₋₁₂cycloalkyl is selected from the group consisting of C₃₋₆monocyclic cycloalkyl, C₈₋₁₂bicyclic cycloalkyl, C₈₋₁₂spiro-cyclic cycloalkyl and C₈₋₁₂bridged cycloalkyl.

When a particular group is “substituted” (e.g. alkyl, cycloalkyl, phenyl, etc.), that group may have one or more substituents, preferably from one to five substituents, more preferably from one to three substituents, most preferably from one to two substituents, independently selected from the list of substituents.

With reference to substituents, the term “independently” means that when more than one of such substituents is possible, such substituents may be the same or different from each other.

Under standard nomenclature used throughout this disclosure, the terminal portion of the designated side chain is described first, followed by the adjacent functionality toward the point of attachment. Thus, for example, a “phenylC₁-₆alkylaminocarbonylC₁-C₆alkyl” substituent refers to a group of the formula

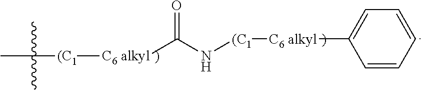

Abbreviations used in the specification, particularly the Schemes and Examples, are as listed in Table A, below.

TABLE A
Abbreviations

| | |
|---|---|
| Boc or BOC = | tert-Butoxycarbonyl |
| BSA = | Bovine serum Albumin |
| t-BuOH or tert-BuOH = | tert-Butanol |
| Burgess reagent = | (Methoxycarbonylsulfamoyl)triethylammonium |
| | hydroxide, inner salt |
| cAMP = | Cyclic adenosine monophosphate |
| CB1 = | Cannabinoid Receptor 1 |
| CB2 = | Cannabinoid Receptor 2 |
| CNS = | Central Nervous System |
| conc. = | Concentrated |
| Cu(OAc)₂ = | Copper Acetate |
| DCE = | 1,1-Dichloroethane |
| DCM = | Dichloromethane |
| DIPEA or DIEA = | Diisopropylethylamine |
| DME = | Dimethoxyethane |
| DMF = | N,N-Dimethylformamide |
| DMF-DMA = | N,N-Dimethylformamide Dimethylacetal |
| DMSO = | Dimethylsulfoxide |
| dppf = | 1,1′-Bis(diphenylphosphino)ferrocene |
| EDCI = | 1-Ethyl-3-(3-dimetylaminopropyl)carbodiimide |
| EtOH = | Ethanol |
| Et₂O = | Diethyl Ether |
| Et₃N or TEA = | Triethylamine |
| EtOAc or EA = | Ethyl acetate |
| FBS = | Fetal Bovine Serum |
| HATU = | O-(7-Azabenzotriazol-1-yl)-N,N,N″,N″- |
| | Tetramethyl Uronium Hexafluorophosphate |
| HBSS = | Hank's Balanced Salt Solution |
| HEPES = | 4-(2-hydroxyethyl)-1-piperazineethanesulfonic |
| | acid) |
| HOBT or HOBt = | 1-Hydroxybenzotriazole |
| HPLC = | High Pressure Liquid Chromatography |
| IBMX = | (3-isobutyl-1-methylxanthine) |
| IPA = | Isopropyl Alcohol |
| Lawesson's Reagent = | 2,4-Bis(4-methoxyphenyl)-1,3,2,4- |
| | dithiadiphosphetane-2,4-dithione |
| LC-MS or LCMS = | Liquid chromatography-mass spectrometry |
| LDA = | Lithium diisopropylamide |
| LiHMDS or LHMDS = | Lithium hexamethyldisilazide or Lithium |
| | bis(trimethylsilyl)amide |
| LiTMP = | Lithium tetramethylpiperidide |
| Martin's Sulfurane = | Bis[α,α-bis(trifluoromethyl) |
| | benzenemethanolato]diphenylsulfur |
| mCPBA = | meta-Chloroperoxybenzoic Acid |
| Me = | Methyl (i.e. —CH₃) |
| MeOH = | Methanol |
| MOM = | Methoxy methyl |
| MTBE = | Methyl t-butyl ether |
| NaOt-Bu = | Sodium tert-Butoxide |
| NMR = | Nuclear magnetic resonance |
| PdCl₂(dppf) = | 1,1′-Bis(diphenylphosphino) |
| | ferrocenedichloropalladium(II) |
| Pd(P(o-tol)₃)₂ = | Dichlorobis(tri-o-tolylphosphine)palladium(II) |
| PyBrop = | Bromortripyrrolidinophosphonium |
| | hexafluorophosphate |
| Rh(cod)BF₄ = | Bis(1,5-cyclooctadiene)rhodium(I) |
| | tetrafluoroborate |
| RT or r.t. or rt = | Room Temperature (~20° C.) |
| satd. = | Saturated |
| TEA = | Triethylamine |
| TFA = | Trifluoroacetic Acid |
| TFAA = | Trifluoroacetic acid anhydride |
| THF = | Tetrahydrofuran |
| THP = | Tetrahydropyran |
| TLC = | Thin Layer Chromatography |
| TMS = | Trimethylsilyl |
| pTSA = | para-Toluenesulfonic Acid |

As used herein, the notation “*” shall denote the presence of a stereogenic center.

Where the compounds according to this invention have at least one chiral center, they may accordingly exist as enantiomers. Where the compounds possess two or more chiral centers, they may additionally exist as diastereomers. It is to be understood that all such isomers and mixtures thereof are encompassed within the scope of the present invention. Preferably, wherein the compound is present as an enantiomer, the enantiomer is present at an enantiomeric excess of greater than or equal to about 80%, more preferably, at an enantiomeric excess of greater than or equal to about 90%, more preferably still, at an enantiomeric excess of greater than or equal to about 95%, more preferably still, at an enantiomeric excess of greater than or equal to about 98%, most preferably, at an enantiomeric excess of greater than or equal to about 99%. Similarly, wherein the compound is present as a diastereomer, the diastereomer is present at an diastereomeric excess of greater than or equal to about 80%, more preferably, at an diastereomeric excess of greater than or equal to about 90%, more preferably still, at an diastereomeric excess of greater than or equal to about 95%, more preferably still, at an diastereomeric excess of greater than or equal to about 98%, most preferably, at an diastereomeric excess of greater than or equal to about 99%.

Furthermore, some of the crystalline forms for the compounds of the present invention may exist as polymorphs and as such are intended to be included in the present invention. In addition, some of the compounds of the present invention may form solvates with water (i.e., hydrates) or common organic solvents, and such solvates are also intended to be encompassed within the scope of this invention.

As used herein, unless otherwise noted, the term “isotopologues” shall mean molecules that differ only in their isotopic composition. More particularly, an isotopologue of a molecule differs from the parent molecule in that it contains at least one atom which is an isotope (i.e. has a different number of neutrons from its parent atom). For example, isotopologues of water include, but are not limited to, “light water” (HOH or H₂O), “semi-heavy water” with the deuterium isotope in equal proportion to protium (HDO or ¹H²HO), “heavy water” with two deuterium isotopes of hydrogen per molecule (D₂O or ²H₂O). “super-heavy water” or tritiated water (T₂O or ³H₂O), where the hydrogen atoms are replaced with tritium (³H) isotopes, two heavy-oxygen water isolopologues (H₂¹⁸O and H₂¹⁷O) and isotopologues where the hydrogen and oxygen water isotobologue D₂¹⁸O.

It is intended that within the scope of the present invention, any one or more element(s), in particular when mentioned in relation to a compound of formula (I) or compound of formula (II), shall comprise all isotopes and isotopic mixtures of said element(s), either naturally occurring or synthetically produced, either with natural abundance or in an isotopically enriched form. For example, a reference to hydrogen includes within its scope ¹H, ²H (D), and ³H (T). Similarly, references to carbon and oxygen include within their scope respectively ¹²C, ¹³ C and ¹⁴C and 16O and 18O. The isotopes may be radioactive or non-radioactive. Radio labelled compounds of formula (I) and/or compounds of formula (II) may comprise one or more radioactive isotope(s) selected from the group of ³H, ¹¹C, ¹⁸F, ¹²²I, ¹²³I, ¹²⁵I, ¹³¹I, ⁷⁵Br, ⁷⁶Br, ⁷⁷Br, and ⁸²Br, . Preferably, the radioactive isotope is selected from the group of ³H, ¹¹C and ¹⁸F.

As used herein, unless otherwise noted, the term “isolated form” shall mean that the compound is present in a form which is separate from any solid mixture with another compound(s), solvent system or biological environment. In an embodiment of the present invention, the compound of formula (I) is present in an isolated form. In an embodiment of the present invention, the compound of formula (II) is present in an isolated form.

As used herein, unless otherwise noted, the term “substantially pure form” shall mean that the mole percent of impurities in the isolated compound is less than about 5 mole percent, preferably less than about 2 mole percent, more preferably, less than about 0.5 mole percent, most preferably, less than about 0.1 mole percent. In an embodiment of the present invention, the compound of formula (I) is present as a substantially pure form. In an embodiment of the present invention, the compound of formula (II) is present as a substantially pure form.

As used herein, unless otherwise noted, the term “substantially free of a corresponding salt form(s)” when used to described the compound of formula (I) shall mean that mole percent of the corresponding salt form(s) in the isolated base of formula (I) is less than about 5 mole percent, preferably less than about 2 mole percent, more preferably, less than about 0.5 mole percent, most preferably less than about 0.1 mole percent. In an embodiment of the present invention, the compound of formula (I) is present in a form which is substantially free of corresponding salt form(s). In an embodiment of the present invention, the compound of formula (II) is present in a form which is substantially free of corresponding salt form(s).

As used herein, unless otherwise noted, the terms “treating”, “treatment” and the like, shall include the management and care of a subject or patient (preferably mammal, more preferably human) for the purpose of combating a disease, condition, or disorder and includes the administration of a compound of the present invention to prevent the onset of the symptoms or complications, alleviate the symptoms or complications (including, to reduce the frequency or severity of one or more symptoms), or eliminate the disease, condition, or disorder.

As used herein, unless otherwise noted, the term “prevention” shall include (a) the delay or avoidance of the development of additional symptoms; and/or (b) delay or avoidance of the development of the disorder or condition along a known development pathway.

One skilled in the art will recognize that wherein the present invention is directed to methods of prevention, a subject in need of thereof (i.e. a subject in need of prevention) shall include any subject or patient (preferably a mammal, more preferably a human) who has experienced or exhibited at least one symptom of the disorder, disease or condition to be prevented. A subject in need thereof may additionally be a subject (preferably a mammal, more preferably a human) who has not exhibited any symptoms of the disorder, disease or condition to be prevented, but who has been deemed by a physician, clinician or other medical profession to be at risk of developing said disorder, disease or condition. For example, the subject may be deemed at risk of developing a disorder, disease or condition (and therefore in need of prevention or preventive treatment) as a consequence of the subject's medical history, including, but not limited to, family history, pre-disposition, co-existing (comorbid) disorders or conditions, genetic testing, and the like.

The term “subject” as used herein, refers to an animal, preferably a mammal, most preferably a human, who has been the object of treatment, observation or experiment. Preferably, the subject has experienced and/or exhibited at least one symptom of the syndrome, condition, disease or disorder to be treated and/or prevented.

The term “therapeutically effective amount” as used herein, means that amount of active compound or pharmaceutical agent that elicits the biological or medicinal response in a tissue system, animal or human that is being sought by a researcher, veterinarian, medical doctor or other clinician, which includes alleviation of the symptoms of the disease or disorder being treated.

As used herein, the term “composition” is intended to encompass a product comprising the specified ingredients in the specified amounts, as well as any product which results, directly or indirectly, from combinations of the specified ingredients in the specified amounts.

As more extensively provided in this written description, terms such as “reacting” and “reacted” are used herein in reference to a chemical entity that is any one of: (a) the actually recited form of such chemical entity, and (b) any of the forms of such chemical entity in the medium in which the compound is being considered when named.

One skilled in the art will recognize that, where not otherwise specified, the reaction step(s) is performed under suitable conditions, according to known methods, to provide the desired product. One skilled in the art will further recognize that, in the specification and claims as presented herein, wherein a reagent or reagent class/type (e.g. base, solvent, etc.) is recited in more than one step of a process, the individual reagents are independently selected for each reaction step and may be the same of different from each other. For example, wherein two steps of a process recite an organic or inorganic base as a reagent, the organic or inorganic base selected for the first step may be the same or different than the organic or inorganic base of the second step. Further, one skilled in the art will recognize that wherein a reaction step of the present invention may be carried out in a variety of solvents or solvent systems, said reaction step may also be carried out in a mixture of the suitable solvents or solvent systems.

To provide a more concise description, some of the quantitative expressions given herein are not qualified with the term “about”. It is understood that whether the term “about” is used explicitly or not, every quantity given herein is meant to refer to the actual given value, and it is also meant to refer to the approximation to such given value that would reasonably be inferred based on the ordinary skill in the art, including approximations due to the experimental and/or measurement conditions for such given value.

To provide a more concise description, some of the quantitative expressions herein are recited as a range from about amount X to about amount Y. It is understood that wherein a range is recited, the range is not limited to the recited upper and lower bounds, but rather includes the full range from about amount X through about amount Y, or any amount or range therein.

Examples of suitable solvents, bases, reaction temperatures, and other reaction parameters and components are provided in the detailed descriptions which follow herein. One skilled in the art will recognize that the listing of said examples is not intended, and should not be construed, as limiting in any way the invention set forth in the claims which follow thereafter.

As used herein, unless otherwise noted, the term “aprotic solvent” shall mean any solvent that does not yield a proton. Suitable examples include, but are not limited to DMF, 1,4-dioxane, THF, acetonitrile, pyridine, dichloroethane, dichloromethane, MTBE, toluene, acetone, and the like.

As used herein, unless otherwise noted, the term “leaving group” shall mean a charged or uncharged atom or group which departs during a substitution or displacement reaction. Suitable examples include, but are not limited to, Br, Cl, I, mesylate, tosylate, and the like.

During any of the processes for preparation of the compounds of the present invention, it may be necessary and/or desirable to protect sensitive or reactive groups on any of the molecules concerned. This may be achieved by means of conventional protecting groups, such as those described in Protective Groups in Organic Chemistry, ed, J. E. W. McOmie, Plenum Press, 1973; and T. W. Greene & P. O. M. Wuts, Protective Groups in Organic Synthesis, John Wiley & Sons, 1991. The protecting groups may be removed at a convenient subsequent stage using methods known from the art

As used herein; unless otherwise noted, the term “nitrogen protecting group” shall mean a group which may be attached to a nitrogen atom to protect said nitrogen atom from participating in a reaction and which may be readily removed following the reaction. Suitable nitrogen protecting groups include, but are not limited to carbamates—groups of the formula —C(O)O—R wherein R is for example methyl, ethyl, t-butyl, benzyl, phenylethyl, CH₂—CH—CH₂—, and the like; amides—groups of the formula —C(O)—R′ wherein R′ is for example methyl, phenyl, trifluoromethyl, and the like; N-sulfonyl derivatives—groups of the formula —SO₂-R″ wherein R″ is for example tolyl, phenyl, trifluoromethyl, 2,2,5,7,8-pentamethylchroman-6-yl-, 2,3,6-trimethyl-4-methoxybenzene, and the like. Other suitable nitrogen protecting groups may be Measured: in texts such as T. W. Greene & P. G. M. Wuts, Protective Groups in Organic Synthesis, John Wiley & Sons, 1991.

As used herein, unless otherwise noted, the term “oxygen protecting group” shad mean a group which may be attached to a oxygen atom to protect said oxygen atom from participating in a reaction and which may be readily removed following the reaction. Suitable oxygen protecting groups include, but are not limited to, acetyl, benzoyl, t-butyl-dimethylsilyl, trimethylsilyl (TMS), MOM, THP, and the like. Other suitable oxygen protecting groups may be Measured: in texts such as T. W. Greene & P. G. M. Wuts. Protective Groups in Organic Synthesis, John Wiley & Sons, 1991.

One skilled in the art will recognize that wherein a reaction step of the present invention may be carried out in a variety of solvents or solvent systems, said reaction step may also be carried out in a mixture of the suitable solvents or solvent systems.

Where the processes for the preparation of the compounds according to the invention give rise to mixture of stereoisomers, these isomers may be separated by conventional techniques such as preparative chromatography. The compounds may be prepared in racemic form, or individual enantiomers may be prepared either by enantiospecific synthesis or by resolution. The compounds may, for example, be resolved into their component enantiomers by standard techniques, such as the formation of diastereomeric pairs by salt formation with an optically active acid, such as (−)-di-p-toluoyl-D-tartaric acid and/or (+)-di-p-toluoyl-L-tartaric acid followed by fractional crystallization and regeneration of the free base. The compounds may also be resolved by formation of diastereomeric esters or amides, followed by chromatographic separation and removal of the chiral auxiliary. Alternatively, the compounds may be resolved using a chiral HPLC column.

Additionally, chiral HPLC against a standard may be used to determine percent enantiomeric excess (% ee). The enantiomeric excess may be calculated as follows

[(Rmoles−Smoles)/(Rmoles+Smoles)]×100%

where Rmoles and Smoles are the R and S mole fractions in the mixture such that Rmoles+Smoles=1. The enantiomeric excess may alternatively be calculated from the specific rotations of the desired enantiomer and the prepared mixture as follows:

ee=([α-obs]/[α-max])×100.

The present invention includes within its scope prodrugs of the compounds of this invention. In general, such prodrugs will be functional derivatives of the compounds which are readily convertible in vivo into the required compound. Thus, in the methods of treatment of the present invention, the term “administering” shall encompass the treatment of the various disorders described with the compound specifically disclosed or with a compound which may not be specifically disclosed, but which converts to the specified compound in vivo after administration to the patient. Conventional procedures for the selection and preparation of suitable prodrug derivatives are described, for example, in “Design of Prodrugs”, ed. H. Bundgaard, Elsevier, 1985.

For use in medicine, the salts of the compounds of this invention refer to non-toxic “pharmaceutically acceptable salts.” Other salts may, however, be useful in the preparation of compounds according to this invention or of their pharmaceutically acceptable salts. Suitable pharmaceutically acceptable salts of the compounds include acid addition salts which may, for example, be formed by mixing a solution of the compound with a solution of a pharmaceutically acceptable acid such as hydrochloric acid, sulfuric acid, fumaric acid, maleic acid, succinic acid, acetic acid, benzoic acid, citric acid, tartaric acid, carbonic acid or phosphoric acid. Furthermore, where the compounds of the invention carry an acidic moiety, suitable pharmaceutically acceptable salts thereof may include alkali metal salts, e.g., sodium or potassium salts; alkaline earth metal salts, e.g., calcium or magnesium salts; and salts formed with suitable organic ligands, e.g., quaternary ammonium salts. Thus, representative pharmaceutically acceptable salts include, but are not limited to, the following: acetate, benzenesulfonate, benzoate, bicarbonate, bisulfate, bitartrate, borate, bromide, calcium edetate, camsylate, carbonate, chloride, clavulanate, citrate, dihydrochloride, edetate, edisylate, estolate, esylate, fumarate, gluceptate, gluconate, glutamate, alycollylarsanilate, hexylresorcinate, hydrabamine, hydrobromide, hydrochloride, hydroxynaphthoate, iodide, isothionate, lactate, lactobionate, laurate, malate, maleate, mandelate, mesylate, methylbromide, methylnitrate, methylsulfate, mutate, napsylate, nitrate, N-methylglucamine ammonium salt, oleate, pamoate (embonate), palmitate, pantothenate, phosphate/diphosphate, polygalacturonate, salicylate, stearate, sulfate, subacetate, succinate, tannate, tartrate, teoclate, tosylate, triethiodide and valerate.

Representative acids which may be used in the preparation of pharmaceutically acceptable salts include, but are not limited to, the following: acids including acetic acid, 2,2-dichloroacetic acid, acylated amino acids, adipic acid, alginic acid, ascorbic acid, L-aspartic acid, benzenesulfonic acid, benzoic acid, 4-acetamidobenzoic acid, (+)-camphoric acid, camphorsulfonic acid, (+)-(1S)-camphor-10-sulfonic acid, capric acid, caproic acid, caprylic acid, cinnamic acid, citric acid, cyclamic acid, dodecylsulfuric acid, ethane-1,2-disulfonic acid, ethanesulfonic acid, 2-hydroxy-ethanesulfonic acid, formic acid, fumaric acid, galactaric acid, gentisic acid, glucoheptonic acid, D-gluconic acid, D-glucoronic acid, L-glutamic acid, α-oxo-glutaric acid, glycolic acid, hipuric acid, hydrobromic acid, hydrochloric acid, (+)-L-lactic acid, (±)-DL-lactic acid, lactobionic acid, maleic acid, (−)-L-malic acid, malonic acid, (±)-DL-mandelic acid, methanesulfonic acid, naphthalene-2-sulfonic acid, naphthalene-1,5-disulfonic acid, 1-hydroxy-2-naphthoic acid, nicotinc acid, nitric acid, oleic acid, orotic acid, oxalic acid, palmitic acid, pamoic acid, phosphoric acid, L-pyroglutamic acid, salicylic acid, 4-amino-salicylic acid, sebaic acid, stearic acid, succinic acid, sulfuric acid, tannic acid, (+)-L-tartaric acid, thiocyanic acid, p-toluenesulfonic acid and undecylenic acid.

Representative bases which may be used in the preparation of pharmaceutically acceptable salts include, but are not limited to, the following: bases including ammonia, L-arginine, benethamine, benzathine, calcium hydroxide, choline, deanol, diethanolamine, diethylamine, 2-(diethylarnino)-ethanol, ethanolamine, ethylenediamine, N-methyl-glucamine, hydrabamine, 1H-imidazole, L-lysine, magnesium hydroxide, 4-(2-hydroxyethyl)-morpholine, piperazine, potassium hydroxide, 1-(2-hydroxyethyl) -pyrrolidine, secondary amine, sodium hydroxide, triethanolamine, tromethamine and zinc hydroxide.

### General Synthesis Scheme(s)

Compounds of formula (I) and compounds of formula (II) may be prepared as described in Scheme 1, below.

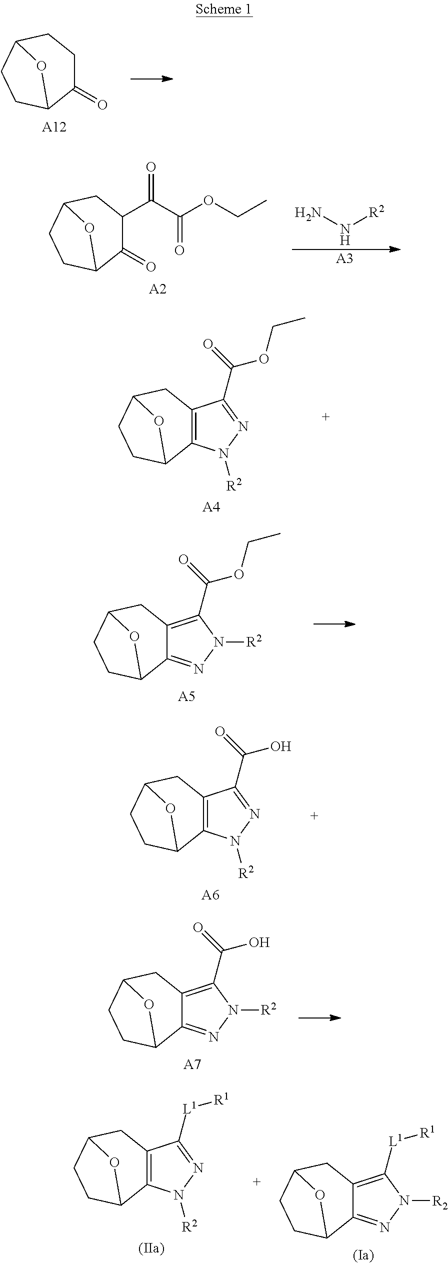

Accordingly, a suitably substituted compound of formula A1, a known compound or compound prepared by known methods is reacted with a suitably selected de-protonating base such as LDA, LiHMDS, LiTMP, and the like; at a temperature in the range of from about −78° C. to about room temperature; in a suitably selected solvent such as THF, Et₂O, DME, and the like; to yield the corresponding metal-enolate (e.g. lithium-enolate) which is then directly reacted with a suitably selected acylating agent such as diethyl oxalate, dimethyl oxalate, ethoxyoxalyl chloride and the like; to yield the corresponding compound of formula A2.

The compound of formula A2 is reacted with a suitably substituted compound of formula A3, wherein R² is optionally substituted phenyl or pyrazin-2-yl, a known compound or compound prepared by known methods; optionally in the presence of a suitably selected acid catalyst such as pTSA, acetic acid, and the like; in a suitably selected organic solvent such as EtOH, 2-propanol, t-BuOH, and the like; at a temperature in the range of from about room temperature to about 150° C.; preferably in the presence of acetic acid, at a temperature of about 90° C.; to yield a mixture of the corresponding compound of formula A4 and the corresponding compound of formula A5.

One skilled in the art will recognize that the compound of formula A2 may alternatively be reacted with a substituted hydrazine, a compound of the formula R²—NH—NH₂, wherein R² is hydrogen or R² is a suitably selected protecting group such as benzyl, BOC, and the like. When R² is hydrogen, the resulting compound is ethyl 1,4,5,6,7,8-hexahydro-5,8-epoxycyclohepta[c]pyrazole-3-carboxylate. When R² is a suitably selected protecting group, the resulting compound may be de-protected, according to known methods, and then further functionalized by reacting with a suitably substituted compound of the formula X—R²; where in X is a suitably selected leaving group such as bromine, chlorine, iodine, triflate, and the like; to yield a mixture of the corresponding compound of formula A4 and the corresponding compound of formula A5.

For example, the mixture of the compound of formula A4 and the compound of formula A5 wherein R² is hydrogen may be reacted with a suitably substituted compound of the formula X—R² wherein R² is an optionally substituted phenyl or pyrazin-2-yl, and wherein X is chloro, bromo or iodo, a known compound or compound prepared by known methods; in the presence of a suitably selected catalyst such as PdCl₂(dppf), Pd (P(o-tol)₃)₂, Rh (cod)BF₄, in a suitably selected solvent such as 1,4-dioxane, DME, THF and the like, in the presence of a base such as NaOt-Bu, Cs₂CO₃, LiHMDS and the like, at a temperature in the range of from room temperature to 120° C., preferably at about 100° C., to yield a mixture of the corresponding compound of formula A4 and formula A5.

Alternatively, the mixture of the compound of formula A4 and the compound of formula A5 wherein R² is hydrogen may be reacted with a suitably substituted compound of the formula X—R², wherein R² is an optionally substituted phenyl or pyrazin-2yl, and wherein X is —B(OH)₂, a known compound or compound prepared by known methods; in the presence of a suitably selected catalyst such as Cu(OAc)₂, NiCl₂·6H₂0, in a suitably selected solvent such as DCM, CH₃CN; in the presence of a base such as Et₃N, DIPEA, and the like, at a temperature in the range of from room temperature to 100° C. to yield a mixture of the corresponding compounds of formula A4 and formula A5.

The mixture of the compound of formula A4 and compound of formula A5 is reacted with a suitably selected hydrolyzing agent such as an aqueous base such as NaOH, LiOH, and the like; in a suitably selected solvent or mixture of solvents such as THF/MeOH, 1,4-dioxane/MeOH, THF, and the like; preferably at about room temperature; to yield a mixture of the corresponding compound of formula A6 and the corresponding compound of formula A7.

The mixture of the compound of formula A6 and the compound of formula A7 is reacted with a suitably substituted secondary amine of the formula R¹—NH₂, where in R¹ is an optionally substituted, C₁₋₆alkyl or a C₃₋₁₀cycloalkyl group, a known compound or compound prepared by known methods; according to known methods, to yield the corresponding compound of formula (IIa) and the corresponding compound of formula (Ia), wherein L¹-R¹ is an secondary alkyl amide (i.e. a group of the formula —C(O)—NH—R¹).

In an example, the acid functionality (e.g. the —C(O)OH group) on the compound of formula A6 and the compound of formula A7 may be reacted with a suitably substituted amine, a compound of the formula R¹—NH₂; in the presence of a suitably selected coupling agent such as 1-(3-dimethylaminopropyl)-3-ethylcarbodiimide hydrochloride (EDCl), 1-hydroxybenzotriazole-6-sulfonamidomethyl hydrochloride (HOBt), bromotripyrrolidinophosphonium hexafluorophosphate (PyBrOP), and the like; in a suitably selected solvent such as DCM, DCE, DMF, and the like; at a temperature in the range of from about −40° C. to about 100° C.; to yield a mixture of the corresponding compound of formula (IIa) and the corresponding compound of formula (Ia), wherein -L¹-R¹ is an secondary alkyl amide (i.e. a group of the formula —C(O)—NH—R¹).

In another example, the acid functionality (e.g. the —C(O)OH group) on the compound of formula A6 and the compound of formula A7 is reacted with a suitably selected chlorinating agent such as oxalyl chloride, thionyl chloride, and the like, in a solvent such as DCM, DCE, THF, and the like; at a temperature ranging from about −20° C. to about room temperature; to yield a mixture of the corresponding acid chloride compounds (i.e. —C(O)Cl derivatives); which intermediate acid chloride compounds are then reacted with a suitably substituted amine of the formula R¹—NH₂; in the presence of a suitably selected base such as triethylamine, diisopropylethyl amine, and the like; in a suitably selected solvent such as DCM, THF, DMF, and the like; at a temperature ranging from about −20° C. to about room temperature; to yield a mixture of the corresponding compound of formula (Ia) and the corresponding compound of formula (Ia) wherein -L¹-R¹ is an secondary alkyl amide (i.e. a group of the formula —C(O)—NH—R¹).

Certain compounds of formula (Ia) and formula (IIa) wherein -L¹-R¹ is other than —C(O)—NH—R¹ may be prepared from the corresponding compound of formula B1 and compound of formula B2

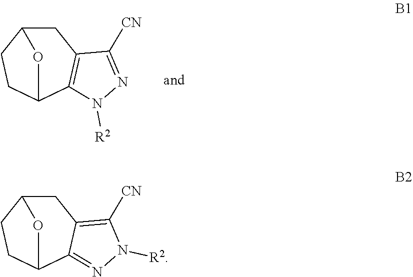

The compounds of formula B1 and formula B2 may be prepared as described in Scheme 2, below.

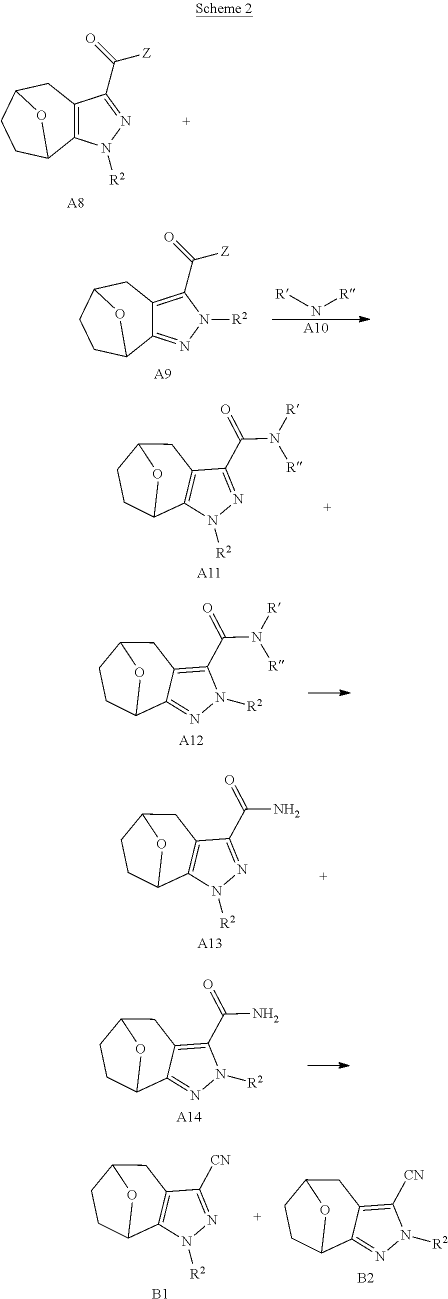

Accordingly, a mixture of suitably substituted compound of formula A8 and suitably substituted compound of formula A9, wherein Z is OH or Cl (i.e. the corresponding acid or acid chloride compounds), prepared for example as described above, is reacted with a suitably substituted compound of formula A10, wherein A10 is ammonia (such that R′ and R″ are both hydrogen), or an ammonia equivalents such as dibenzyl amine (such that R′ and R″ are both benzyl), p-methoxybenylamine (such R′ is hydrogen and R″ is p-methoxybenzyl), benzyl amine (such that R′ is hydrogen and R″ is benzyl), and the like; a known compound or compound prepared by known methods; according to known methods; to yield a mixture of the corresponding compound of formula All and corresponding compound of formula A12.

For example, wherein A10 is p-methoxybenzyamine, the mixture of the compound of formula A8 and formula A9 is reacted with the compound of formula A10; in the presence of suitably selected base such as TEA, DIPEA, pyridine, and the like; in a suitably selected solvent such as DCM, DCE, 1,4-dioxane, and the like. In another example, wherein A10 is ammonia, the mixture of the compound of formula A8 and formula A9, wherein Z is Cl is reacted with A10 (ammonia); in a suitably selected solvent such as THF, and the like, at about 0° C.

The mixture of the compound of formula A11 and the compound of formula A12 is reacted according to known methods; to yield the corresponding compound of formula A13 and the corresponding compound of formula A14. For example, wherein the compound of formula A13 and formula A14 R′ is hydrogen and R″ is p-methoxybenzyl, said mixture is reacted with a suitably selected acid such as TFA, conc. HCl, and the like; in a suitably selected solvent such as DCM. DCE, and the like.

The mixture of the compound of formula A13 and the compound of formula A14 is reacted with a suitably selected dehydrating agent such as trifluoroacetic anhydride, phosphorus oxychloride, thionyl chloride, and the like; in a suitable solvent such as DCM, DCE, and the like; or in an excess of the dehydrating reagent (such that the dehydrating reagent also acts as the solvent); at a temperature in the range of from about 0° C. to about 100° C.; to yield a mixture of the corresponding compound of formula B1 and the corresponding compound of formula B2.

Compounds of formula (I) and formula (Ia) wherein -L¹-R¹ is

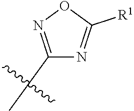

may be prepared as described in Scheme 3, below.

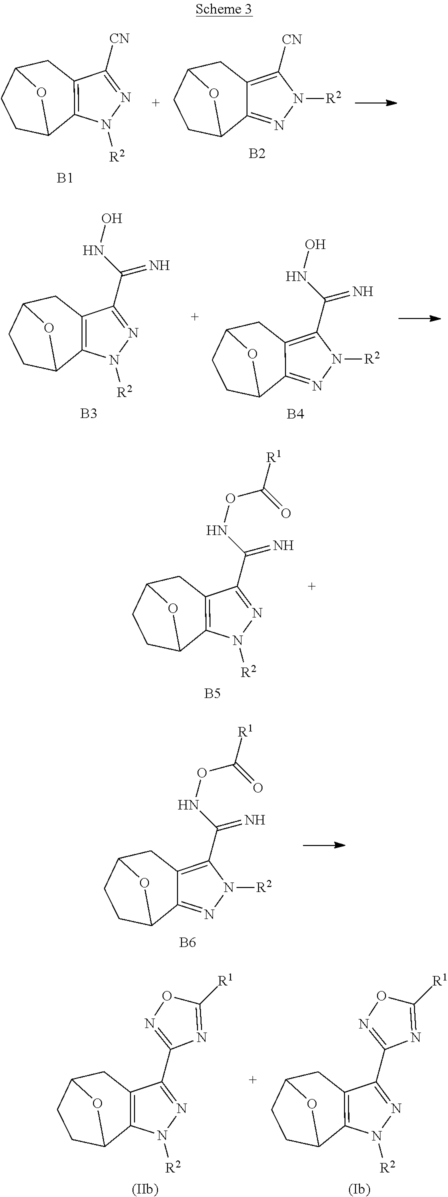

Accordingly, a mixture of suitably substituted compound of formula B1 and formula B2 is reacted with hydroxyl amine, a known compound, in a suitably selected solvent such as tert-BuOH, EtOH, 2-propanol, and the like; at a temperature of about 90° C.; to yield a mixture of the corresponding compound of formula B33 and corresponding compound of formula B4.

The mixture of the compound of formula B3 and formula B4 is reacted with a suitably selected esterification reagent such as a suitably substituted acid chloride of the formula R¹—C(O)—Cl; in the presence of a suitably selected organic base such as TEA, DIPEA, pyridine, and the like; in a suitably selected solvent such as DCM, ICE, THF and the like; to yield a mixture of the corresponding compound of formula B5 and the corresponding compound of formula B6.

The mixture of the compound of formula B5 and formula B6 is reacted under thermal ring closure conditions, for example, heating to a temperature of about 120° C.; in a suitably selected solvent such as toluene, xylene, ethyl benzene, and the like; for a time period sufficient to effect the ring closure; to yield a mixture of the corresponding compounds of formula (IIb) and formula (Ib) (compounds of formula (IIa) and formula (Ia) wherein -L¹-R¹ is

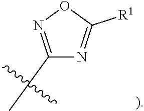

Compounds of formula (Ia) and compounds of formula (IIa) wherein -L¹-R¹ is

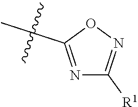

may be prepared as described in Scheme 4 below.

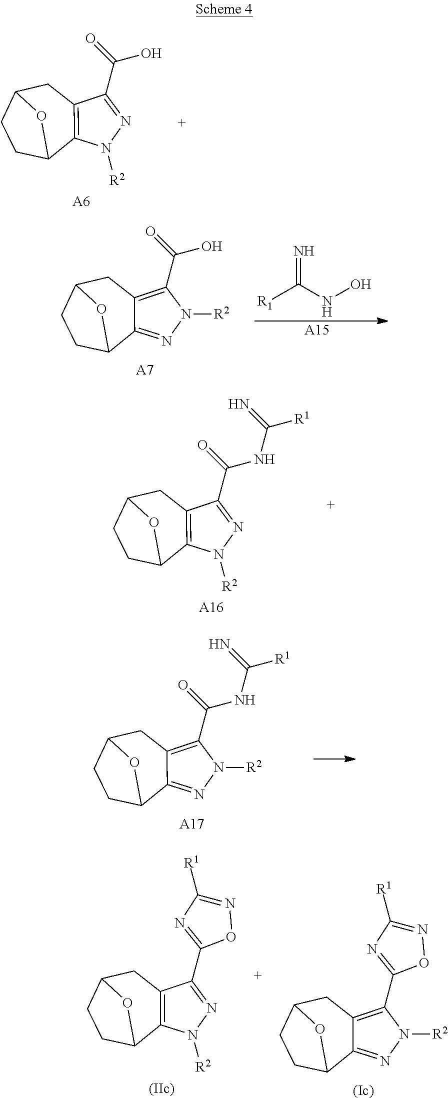

Accordingly, a mixture of suitably substituted compound of formula A6 and formula A7 is reacted with a suitably substituted compound of formula A15, a known compound or compound prepared by known methods; in the presence of a suitably selected coupling agent such 1-(3-dimethylaminopropyl)-3-ethylcarbodiimide hydrochloride (EDCl), 1-hydroxybenzotriazole-6-sulfonamidomethyl hydrochloride (HOBt), bromotripyrrolidinophosphonium hexafluorophosphate (PyBrOP), and the like; in a suitably selected solvent such as DCM, DCE, THF and the like; to yield a mixture of the corresponding compound of formula A16 and the corresponding compound of formula A17.

The mixture of compound of formula A16 and formula A17 is subjected to heating to effect thermal ring closure, for example heating the mixture of compound of formula A16 and formula at about 120° C.; in a suitably selected solvent such as toluene, ethyl benzene, xylene and the like; to yield a mixture of the corresponding compounds of formula (IIc) and corresponding compound of formula (Ic) (compounds of formula (Ia) and formula (Ia) wherein L¹-R¹ is

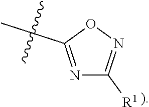

Compounds of (Ia) and compounds of formula (IIa) wherein -L¹-R¹ is

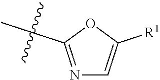

may be prepared as described in Scheme 5, below.

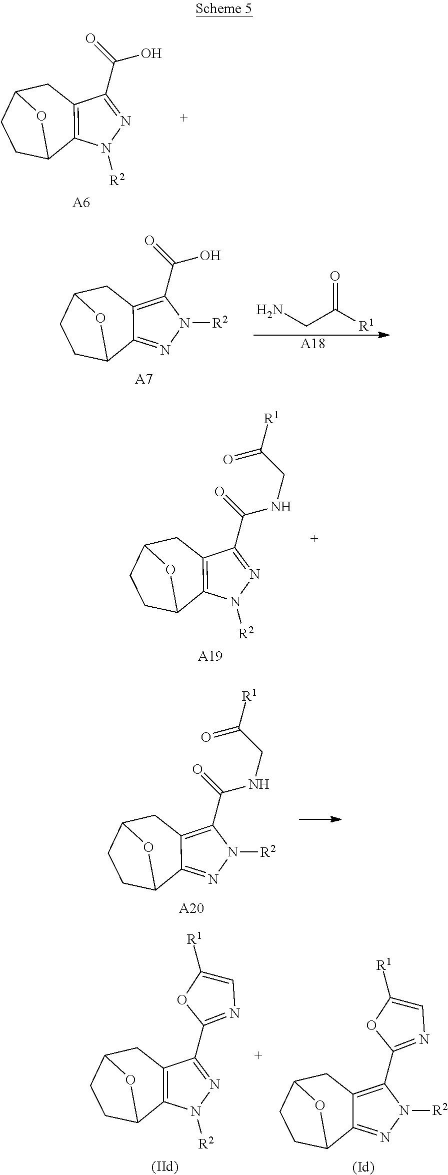

Accordingly, a mixture of suitably substituted compound of formula A6 and formula A7 is reacted with a suitably substituted compound of formula A18, a known compound or compound prepared by known methods; in the presence of a suitably selected coupling agent such as 1-(3-dimethylaminopropyl)-3-ethylcarbodiimide hydrochloride (EDCl), 1-hydroxybenzotriazole-6-sulfonamidomethyl hydrochloride (HOBt), bromotripyrrolidinophosphonium hexafluorophosphate (PyBrOP), and the like; in the presence of a suitably selected base such as DIPEA, TEA, TIPA, and the like; in a suitably selected solvent such as DCM, DCE, DMF, and the like; to yield a mixture of the corresponding compound of formula A19 and the corresponding compound of formula A20.

The mixture of the compound of formula A19 and formula A20 is reacted with a suitably selected dehydrating reagent such as Burgess reagent, Martin's sulfurane, trifluoracetic anhydride, and the like; in a suitably selected solvent such as toluene, ethylbenzene, xylene, and the like; at a temperature of about 110° C.; to yield a mixture of the corresponding compound of formula (IId) and the corresponding compound of formula (Id) (compounds of formula (I) and formula (II) wherein -L¹-R¹ is

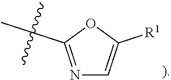

Compounds of formula (Ia) and compounds of formula (IIa) wherein -L¹-R¹ is

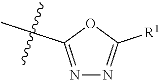

may be prepared as described in Scheme 6, below.

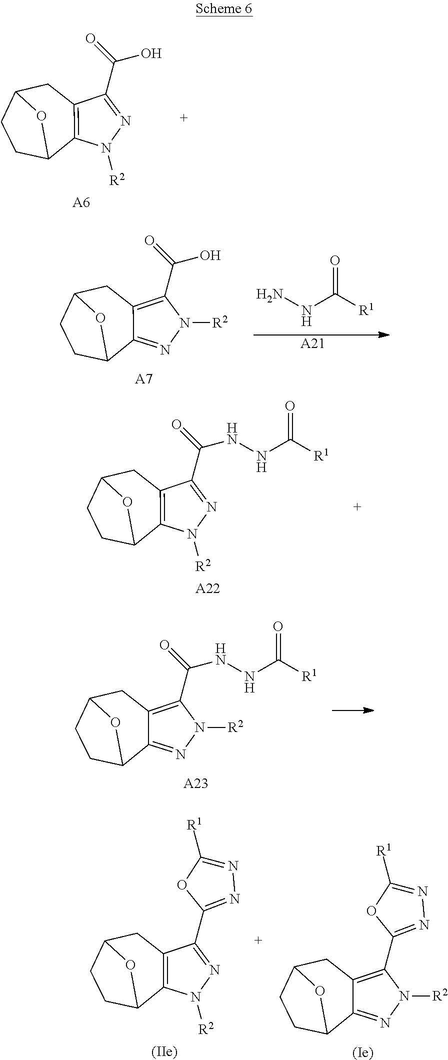

Accordingly, a mixture of suitably substituted compound of formula A6 and formula A7 is reacted with a suitably substituted compound of formula A21, a known compound or compound prepared by known methods, in the presence of a suitably selected coupling agent such as 1-(3-dimethylaminopropyl)-3-ethylcarbodiimide hydrochloride (EDCl), 1-hydroxybenzotriazole-6-sulfonamidomethyl hydrochloride (HOBt), bromotripyrrolidinophosphonium hexafluorophosphate (PyBrOP), and the like; in the presence of a suitably selected base such as DIPEA, TEA, TIPA, and the like; in a suitably selected solvent such as DCM, DCE, DMF, and the like; to yield a mixture of the corresponding compound of formula A22 and corresponding compound of formula A23.

The mixture of the compound of formula A22 and formula A23 is reacted with a suitably selected dehydrating reagent such as Burgess reagent, Martin's sulfurane, trifluoracetic anhydride, and the like; in a suitably selected solvent such as toluene, ethylbenzene, xylene, and the like; at a temperature of about 110° C.; to yield a mixture of the corresponding compound of formula (IIe) and corresponding compound of formula (Id) (compounds of formula (Ia) and (IIa) wherein -L¹-R¹ is

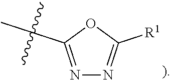

Compounds of formula (Ia) and compounds of formula (IIa) wherein -L¹-R¹ is

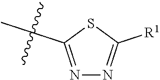

may be prepared as described in Scheme 7, below.

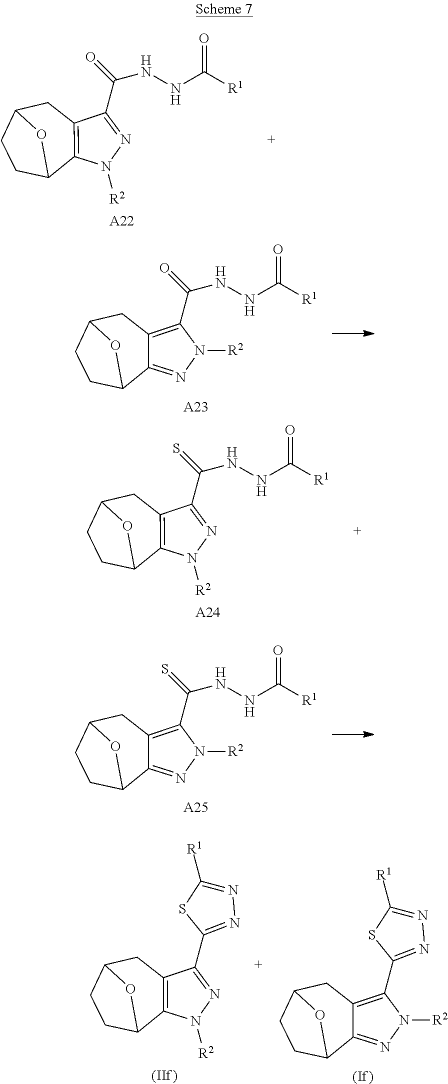

Accordingly, a mixture of suitably substituted compound of formula A22 and formula A23 is reacted with a suitably selected thiolating reagent such as Lawesson's reagent, POSCl₃, elemental S, and the like, preferably Lawesson's reagent; in a solvent such as 1,4-dioxane, THF, DME, and the like; at a temperature of for example, about 100° C.; to yield a mixture of the corresponding compound of formula A24 and the corresponding compound of formula A25.

The mixture of compound of formula A24 and formula A25 is reacted to effect thermal cyclization, for example heating to about 120° C.; in a solvent such as toluene, ethylbenzene, xylene, and the like; to yield a mixture of the corresponding compound of formula (IIf) and the corresponding compound of formula (If) (compounds of formula (Ia) and formula (IIa) wherein -L¹-R¹ is

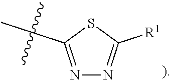

Compounds of formula (Ia) and compounds of formula (IIa) wherein -L¹-R¹ is

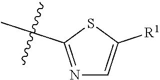

may be prepared as described in Scheme 8, below.

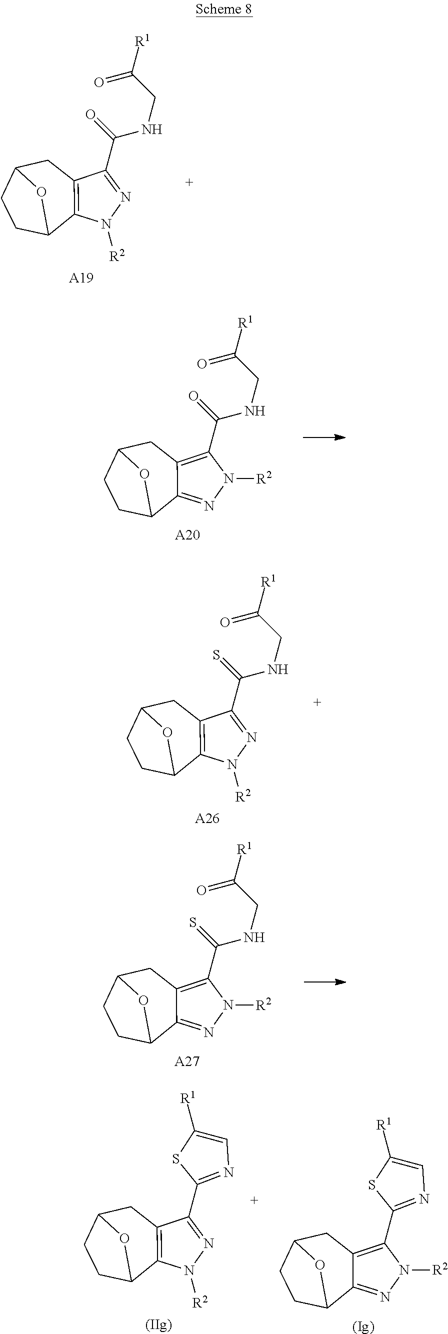

Accordingly, a mixture of suitably substituted compound of formula A19 and formula A20, prepared for example as described herein is reacted with a suitably selected thiolating reagent such as Lawesson's reagent, POSCl₃, elemental S, and the like, preferably Lawesson's reagent; in a solvent such as 1,4-dioxane, THF, DME, and the like; at a temperature of, for example about 100° C.; to yield a mixture of the corresponding compound of formula A26 and the corresponding compound of formula A27.

The mixture of the compound of formula A26 and the compound of formula A27 is reacted to effect thermal cyclization, for example heating to about 120° C.; in a solvent such as toluene, ethylbenzene, xylene, and the like; to yield a mixture of the corresponding compound of formula (IIg) and the corresponding compound of formula (Ig) (compounds of formula (Ia) and formula (IIa) wherein -L¹-R¹ is

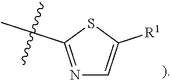

Compounds of formula (Ia) and compounds of formula (IIa) wherein -L¹-R¹ is

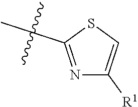

may alternatively be prepared as described in Scheme 9, below.

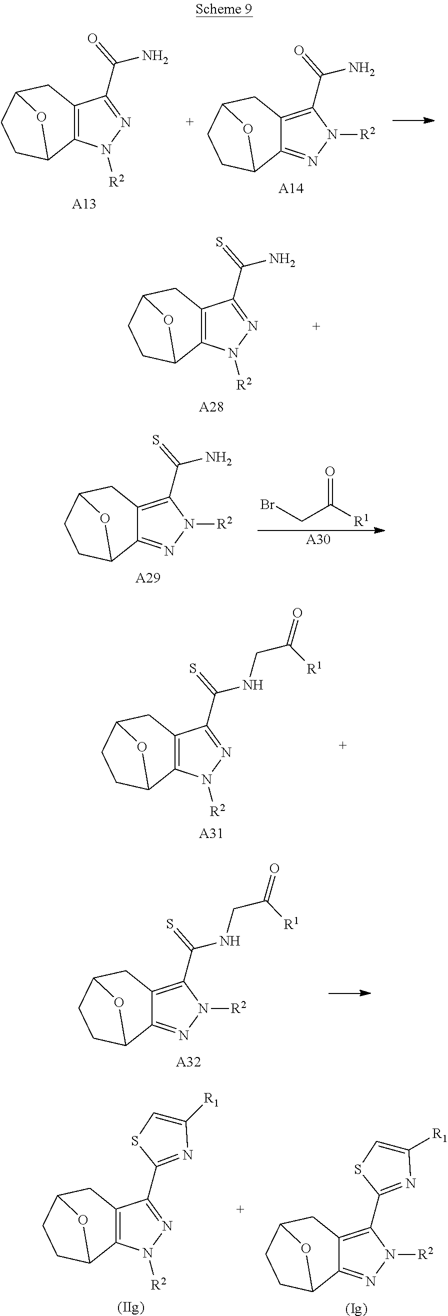

Accordingly, a mixture of suitably substituted compound of formula A13 and formula A14 is reacted with a suitably selected thiolation agent such as Lawesson's reagent, POSCl₃, elemental S, and the like, preferably Lawesson's reagent; in a solvent such as 1,4-dioxane, THF, DME, and the like; at a temperature of, for example, about 100° C.; to yield a mixture of the corresponding compound of formula A28 and the corresponding compound of formula A29.

The mixture of compound of formula A28 and formula A29 is reacted with a suitably substituted compound of formula A30, a known compound or compound prepared according to known methods; in s suitably selected solvent such as EtOH, propanol, tert-BuOH and the like; at an elevated temperature in the range of from about 70° C. to about 100° C.; to yield a mixture of the corresponding compound of formula A31 and the corresponding compound of formula A32.

The mixture of compound of formula A31 and formula A32 is reacted to effect thermal cyclization, for example heating to about 70° C.; in a suitably selected solvent such as EtOH, propanol, tert-BuOH and the like; to yield a mixture of the corresponding compound of formula (IIg) and the corresponding compound of formula (Ig) (compounds of formula (Ia) and formula (IIa) wherein -L¹-R¹ is

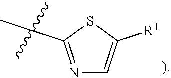

Compounds of formula (Ia) and compounds of formula (IIa) wherein -L¹-R¹ is

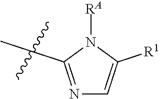

may be prepared as described in Scheme 10, below.

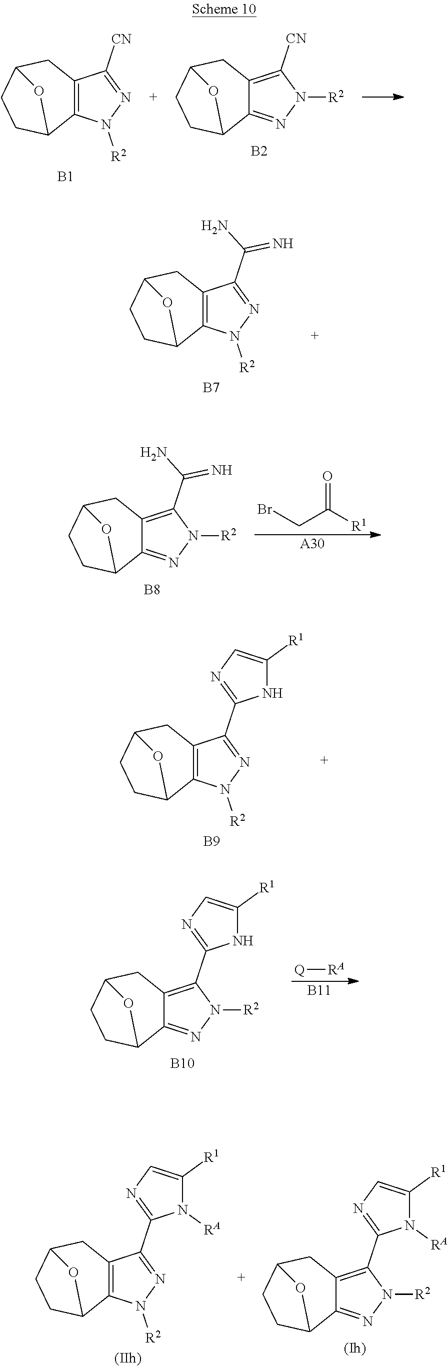

Accordingly, a mixture of suitably substituted compound of formula B1 and formula B2 is reacted with ammonia, according to known methods, for example, reacting the mixture of compound of formula B1 and formula B2 with a suitably selected acid such as conc. HCl, H₂SO₄, and the like; in a suitably selected solvent such as methanol, ethanol, IPA, and the like; to yield the corresponding imidate intermediate; which are then reacted with ammonia; to yield a mixture of the corresponding compound of formula B7 and the corresponding compound of formula B8.

The mixture of compound of formula B7 and formula B8 is reacted with a suitably substituted compound of formula A30, a known compound or compound prepared by known methods; in the presence of a suitably selected base such as K₂CO₃, Na₂CO₃, Cs₂CO₃, and the like; in a suitably selected solvent or mixture of solvent such as THF/water, 1,4-dioxane/H₂O, DME/H₂O and the like; at an elevated temperature in the range of from about 70° C. to about 100° C., for example at about 70° C.; to yield a mixture of the corresponding compound of formula B9 and the corresponding compound of formula B10.

The mixture of compounds of formula B9 and formula B10 is reacted with a suitably selected base such as NaH, LHMDS, LDA, and the like; in a suitably selected solvent such as THF, DMF, DME, and the like; to yield the corresponding de-protonated compound, as its corresponding salt; which is then reacted with a suitably substituted compound of formula B11, wherein Q is a suitably selected leaving group such as Cl, Br, I, and the like, a known compound or compound prepared by known methods, in a suitably selected solvent such as such as THF, DMF, DME, and the like; to yield a mixture of the corresponding compounds of formula (IIh) and the corresponding compound of formula (Ih) (compounds of formula (Ia) and formula (Ia) wherein -L¹-R¹ is

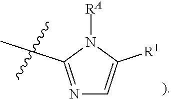

Compounds of formula (I) and compounds of formula (II) wherein R² is pyrazine-1-oxide may be prepared by reacted a mixture of a compound of formula (Ia) and a compound of formula (IIa) wherein R² is pyrazin-2-yl with a suitably selected oxidizing agent such as mCPBA, peracetic acid, urea hydrogen peroxide complex, methylrhenium trioxide and sodium percarbonate, sodium perborate, and the like; in a suitably selected solvent such as DCM, DCE, acetic acid, acetonitrile and the like; at a temperature in the range of from about 0° C. to about 65° C., preferably at about room temperature.

One skilled in the art will further recognize that wherein the compounds of formula (Ia) and formula (IIa) there are other oxidizable groups present (e.g. —NH₂), said oxidizable group(s) may protected prior to oxidation and then de-protected or alternatively may also be oxidized. For example, a thiazole substituent group may be oxidized to the corresponding thiazole-oxide, a thiadiazole may be oxidized to the corresponding thiadiazoleoxide, etc. Thus, one skilled in the art will recognize that compounds of formula (I) and formula (II) wherein -L¹-R¹ is selected from the group consisting of

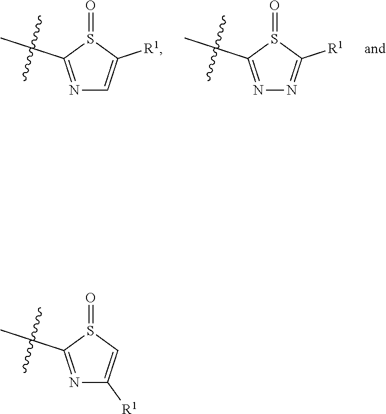

are prepared from the corresponding compound of formula (I) and formula (II) wherein -L¹-R¹ is selected from the group consisting of

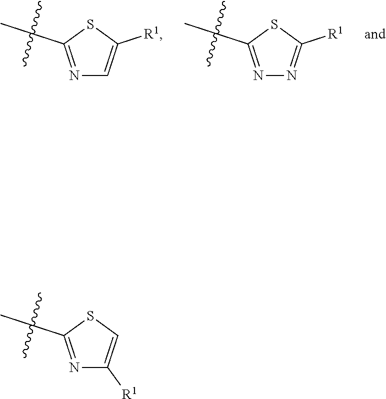

by reacting with a suitably selected oxidizing agent as described above.

One skilled in the art will recognize that wherein a mixture of regio-isomers is prepared, said regio-isomers may be separated at any step of the synthesis, and the individual regio-isomers isolated and/or further reacted a described herein, to yield the desired compound(s) of the present invention. Said separation may be affected according to known methods, for example by preparative HPLC, eluting with a suitably selected solvent such as water/acetonitrile and water/MeOH gradient with additives such ammonium bicarbonate and the like.

One skilled in the art will further recognize that the regio-isomer ratios (for the mixture of compounds prepared as describe in herein) may be achieved by optimizing the reaction conditions.

### Pharmaceutical Compositions

The present invention further comprises pharmaceutical compositions containing one or more compounds of formula (I) and/or compounds of formula (II) with a pharmaceutically acceptable carrier. Pharmaceutical compositions containing one or more of the compounds of the invention described herein as the active ingredient can be prepared by intimately mixing the compound or compounds with a pharmaceutical carrier according to conventional pharmaceutical compounding techniques. The carrier may take a wide variety of forms depending upon the desired route of administration (e.g., oral, parenteral). Thus for liquid oral preparations such as suspensions, elixirs and solutions, suitable carriers and additives include water, glycols, oils, alcohols, flavoring agents, preservatives, stabilizers, coloring agents and the like; for solid oral preparations, such as powders, capsules and tablets, suitable carriers and additives include starches, sugars, diluents, granulating agents, lubricants, binders, disintegrating agents and the like. Solid oral preparations may also be coated with substances such as sugars or be enteric-coated so as to modulate major site of absorption. For parenteral administration, the carrier will usually consist of sterile water and other ingredients may be added to increase solubility or preservation. Injectable suspensions or solutions may also be prepared utilizing aqueous carriers along with appropriate additives.

To prepare the pharmaceutical compositions of this invention, one or more compounds of the present invention as the active ingredient is intimately admixed with a pharmaceutical carrier according to conventional pharmaceutical compounding techniques, which carrier may take a wide variety of forms depending of the form of preparation desired for administration, e.g., oral or parenteral such as intramuscular. In preparing the compositions in oral dosage form, any of the usual pharmaceutical media may be employed. Thus, for liquid oral preparations, such as for example, suspensions, elixirs and solutions, suitable carriers and additives include water, glycols, oils, alcohols, flavoring agents, preservatives, coloring agents and the like; for solid oral preparations such as, for example, powders, capsules, caplets, gelcaps and tablets, suitable carriers and additives include starches, sugars, diluents, granulating agents, lubricants, binders, disintegrating agents and the like. Because of their ease in administration, tablets and capsules represent the most advantageous oral dosage unit form, in which case solid pharmaceutical carriers are obviously employed. If desired, tablets may be sugar coated or enteric coated by standard techniques. For parenterals, the carrier will usually comprise sterile water, through other ingredients, for example, for purposes such as aiding solubility or for preservation, may be included. Injectable suspensions may also be prepared, in which case appropriate liquid carriers, suspending agents and the like may be employed. The pharmaceutical compositions herein will contain, per dosage unit, e.g., tablet, capsule, powder, injection, teaspoonful and the like, an amount of the active ingredient necessary to deliver an effective dose as described above The pharmaceutical compositions herein will contain, per unit dosage unit, e.g., tablet, capsule, powder, injection, suppository, teaspoonful and the like, of from about 0.01 mg to about 1000 mg or any amount or range therein, and may be given at a dosage of from about 0.01 mg/kg/day to about 300 mg/kg/day, or any amount or range therein, preferably from about 0.1 mg/kg/day to about 100 mg/kg/day, or any amount or range therein, preferably from about 0.5 mg/kg/day to about 50 mg/kg/day, preferably from about 1.0 mg/kg/day to about 25 mg/kg/day, or any amount or range therein. The dosages, however, may be varied depending upon the requirement of the patients, the severity of the condition being treated and the compound being employed. The use of either daily administration or post-periodic dosing may be employed.

Preferably these compositions are in unit dosage forms from such as tablets, pills, capsules, powders, granules, sterile parenteral solutions or suspensions, metered aerosol or liquid sprays, drops, ampoules, autoinjector devices or suppositories; for oral parenteral, intranasal, sublingual or rectal administration, or for administration by inhalation or insufflation. Alternatively, the composition may be presented in a form suitable for once-weekly or once-monthly administration; for example, an insoluble salt of the active compound, such as the decanoate salt, may be adapted to provide a depot preparation for intramuscular injection. For preparing solid compositions such as tablets, the principal active ingredient is mixed with a pharmaceutical carrier, e.g. conventional tableting ingredients such as corn starch, lactose, sucrose, sorbitol, talc, stearic acid, magnesium stearate, dicalcium phosphate or gums, and other pharmaceutical diluents, e.g. water, to form a solid pre-formulation composition containing a homogeneous mixture of a compound of the present invention, or a pharmaceutically acceptable salt thereof. When referring to these pre-formulation compositions as homogeneous, it is meant that the active ingredient is dispersed evenly throughout the composition so that the composition may be readily subdivided into equally effective dosage forms such as tablets, pills and capsules. This solid pre-formulation composition is then subdivided into unit dosage forms of the type described above containing from about 0.01 mg to about 1,000 mg, or any amount or range therein, of the active ingredient of the present invention. The tablets or pills of the novel composition can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permits the inner component to pass intact into the duodenum or to be delayed in release. A variety of material can be used for such enteric layers or coatings, such materials including a number of polymeric acids with such materials as shellac, cetyl alcohol and cellulose acetate.

The liquid forms in which the novel compositions of the present invention may be incorporated for administration orally or by injection include, aqueous solutions, suitably flavored syrups, aqueous or oil suspensions, and flavored emulsions with edible oils such as cottonseed oil, sesame oil, coconut oil or peanut oil, as well as elixirs and similar pharmaceutical vehicles. Suitable dispersing or suspending agents for aqueous suspensions, include synthetic and natural gums such as tragacanth, acacia, alginate, dextran, sodium carboxymethylcellulose, methylcellulose, polyvinyl-pyrrolidone or gelatin.

The method(s) of treating disorders as described herein may also be carried out using a pharmaceutical composition comprising any of the compounds as defined herein and a pharmaceutically acceptable carrier. The pharmaceutical composition may contain between about 0.01 mg and about 1000 mg of the compound, or any amount or range therein; preferably from about 1.0 mg to about 500 mg of the compound, or any amount or range therein, and may be constituted into any form suitable for the mode of administration selected. Carriers include necessary and inert pharmaceutical excipients, including, but not limited to, binders, suspending agents, lubricants, flavorants, sweeteners, preservatives, dyes, and coatings. Compositions suitable for oral administration include solid forms, such as pills, tablets, caplets, capsules (each including immediate release, timed release and sustained release formulations), granules, and powders, and liquid forms, such as solutions, syrups, elixirs, emulsions, and suspensions. Forms useful for parenteral administration include sterile solutions, emulsions and suspensions.

Advantageously, compounds of the present invention may be administered in a single daily dose, or the total daily dosage may be administered in divided doses of two, three or four times daily. Furthermore, compounds for the present invention can be administered in intranasal form via topical use of suitable intranasal vehicles, or via transdermal skin patches well known to those of ordinary skill in that art. To be administered in the form of a transdermal delivery system, the dosage administration will, of course, be continuous rather than intermittent throughout the dosage regimen.

For instance, for oral administration in the form of a tablet or capsule, the active drug component can be combined with an oral, non-toxic pharmaceutically acceptable inert carrier such as ethanol, glycerol, water and the like. Moreover, when desired or necessary, suitable binders; lubricants, disintegrating agents and coloring agents can also be incorporated into the mixture. Suitable binders include, without limitation, starch, gelatin, natural sugars such as glucose or beta-lactose, corn sweeteners, natural and synthetic gums such as acacia, tragacanth or sodium oleate, sodium stearate, magnesium stearate, sodium benzoate, sodium acetate, sodium chloride and the like. Disintegrators include, without limitation, starch, methyl cellulose, agar, bentonite, xanthan gum and the like.

The liquid forms in suitably flavored suspending or dispersing agents such as the synthetic and natural gums, for example, tragacanth, acacia, methyl-cellulose and the like. For parenteral administration, sterile suspensions and solutions are desired. Isotonic preparations which generally contain suitable preservatives are employed when intravenous administration is desired.

To prepare a pharmaceutical composition of the present invention, a compound of formula (I) and/or a compound of formula (II), as the active ingredient(s), is intimately admixed with a pharmaceutical carrier according to conventional pharmaceutical compounding techniques, which carrier may take a wide variety of forms depending of the form of preparation desired for administration (e.g. oral or parenteral). Suitable pharmaceutically acceptable carriers are well known in the art. Descriptions of some of these pharmaceutically acceptable carriers may be found in The Handbook of Pharmaceutical Excipients, published by the American Pharmaceutical Association and the Pharmaceutical Society of Great Britain.

Methods of formulating pharmaceutical compositions have been described in numerous publications such as Pharmaceutical Dosage Forms: Tablets, Second Edition, Revised and Expanded, Volumes 1-3, edited by Lieberman et al; Pharmaceutical Dosage Forms: Parenteral Medications, Volumes 1-2, edited by Avis et al; and Pharmaceutical Dosage Forms: Disperse Systems, Volumes 1-2, edited by Lieberman et al; published by Marcel Dekker, Inc.

Compounds of this invention may be administered in any of the foregoing compositions and according to dosage regimens established in the art whenever treatment of disorders, as described herein, is required.

The daily dosage of the products may be varied over a wide range from about 0.01 mg to about 1,000 mg per adult human per day, or any amount or range therein. For oral administration, the compositions are preferably provided in the form of tablets containing, 0.01, 0.05, 0.1, 0.5, 1.0, 2.5, 5.0, 10.0, 15.0, 25.0, 50.0, 100, 150, 200, 250 and 500 milligrams of the active ingredient for the symptomatic adjustment of the dosage to the patient to be treated. An effective amount of the drug is ordinarily supplied at a dosage level of from about 0.01 mg/kg to about 300 mg/kg of body weight per day, or any amount or range therein. Preferably, the range is from about 0.1 to about 100.0 mg/kg of body weight per day, or any amount or range therein. More preferably, from about 0.5 to about 50.0 mg/kg of body weight per day, or any amount or range therein. More preferably, from about 1.0 to about 25.0 mg/kg of body weight per day, or any amount or range therein. The compounds may be administered on a regimen of 1 to 4 times per day.

Optimal dosages to be administered may be readily determined by those skilled in the art, and will vary with the particular compound used, the mode of administration, the strength of the preparation, the mode of administration, and the advancement of the disease condition. In addition, factors associated with the particular patient being treated, including patient age, weight, diet and time of administration, will result in the need to adjust dosages.

One skilled in the art will recognize that, both in vivo and in vitro trials using suitable, known and generally accepted cell and/or animal models are predictive of the ability of a test compound to treat or prevent a given disorder.

One skilled in the art will further recognize that human clinical trials including first-in-human, dose ranging and efficacy trials, in healthy patients and/or those suffering from a disorder, may be completed according to methods well known in the clinical and medical arts.

### EXAMPLES

Representative compounds of formula (I) and/or compounds of formula (II) of the present invention were prepared according to the synthesis methods described herein.

The following Examples are set forth to aid in the understanding of the invention and are not intended and should not be construed to limit in any way the invention set forth in the claims which follow thereafter.

In the Examples which follow, some synthesis products are listed as having been isolated as a residue. It will be understood by one of ordinary skill in the art that the term “residue” does not limit the physical state in which the product was isolated and may include, for example, a solid, an oil, a foam, a gum, a syrup, and the like.

### Example 1

### N-(tert-butyl)-1-(2,4-difluorophenyl)-1,4,5,6,7,8-hexahydro-5,8-epoxycyclohepta[c]pyrazole-3-carboxamide (Cpd. 1) and N-(tert-butyl)-2-(2,4-difluorophenyl)-2,4,5,6,7,8-hexahydro-5,8-epoxycyclohepta[c]pyrazole-3-carboxamide (Cpd. 2)

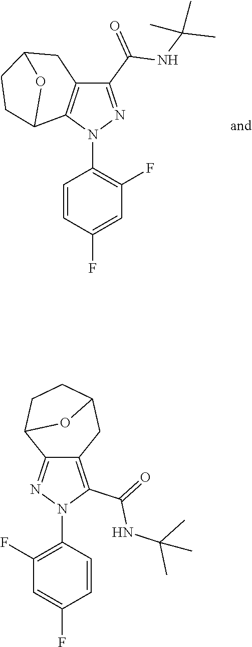 [SMILES: CC(C)(C)NC(=O)c1nn(-c2ccc(F)cc2F)c2c1CC1CCC2O1 ; CC(C)(C)NC(=O)c1c2c(nn1-c1ccc(F)cc1F)C1CCC(C2)O1]

### A. Ethyl 2-oxo-2-(2-oxo-8-oxabicyclo[3.2.1]octan-3-yl)acetate (1A)

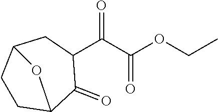 [SMILES: CCOC(=O)C(=O)C1CC2CCC(O2)C1=O]

To a solution of 8-oxa-bicyclo[3.2.1]octan-2-one (300 mg, 2.3 mmol) in tetrahydrofuran (5 mL) was added lithium bis(trimethylsilyl)amide (597 mg, 3.5 mmol). The mixture was stirred at −78 for 30 min, and diethyl oxalate (417 mg, 2.8 mmol) was added. The reaction mixture was allowed to warm to RT and stirred at rt overnight. The reaction was quenched with H₂O (50 mL). The resulting mixture was extracted with EtOAc (3×100 mL). The organic layers were combined, dried over Na₂SO₄, filtered and concentrated. The resulting residue was purified by silica gel chromatography (0-10% EtOAc/petroleum ether) to yield the title compound. Mass Spectrum (LCMS. ESI pos.): Calculated: for C₁₁H₁₄O₅: 227.08 (M+H), Measured: 227.1.

### B. Ethyl 1-(2,4-difluorophenyl)-1,4,5,6,7,8-hexahydro-5,8-epoxycyclohepta[c]pyrazole-3-carboxylate (1B) and Ethyl 2-(2,4-difluorophenyl) -2,4,5,6,7,8-hexahydro-5,8-epoxycyclohepta[c]pyrazole-3-carboxylate (1B′)

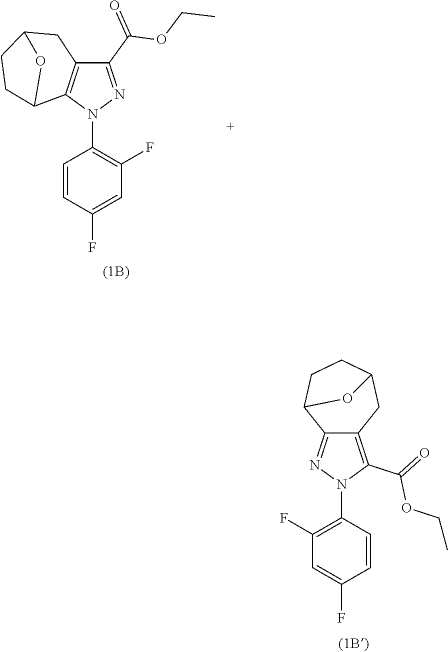 [SMILES: CCOC(=O)c1nn(-c2ccc(F)cc2F)c2c1CC1CCC2O1 ; CCOC(=O)c1c2c(nn1-c1ccc(F)cc1F)C1CCC(C2)O1]

To a solution of Ethyl 2-oxo-2-(2-oxo-8-oxabicyclo[3.2.1]octan-3-yl)acetate (1A) (200.0 mg, 0.884 mmol) in glacial acetic acid glacial (5 mL) was added (2,4-difluorophenyphydrazine (127 mg, 0.884 mmol). The mixture was stirred at 90° C. for 3 h. The reaction mixture was allowed to cool to RT and concentrated, and dried to yield the ethyl 1-(2,4-difluorophenyl)-1,4,5,6,7,8-hexahydro-5,8-epoxycyclohepta[c]pyrazole-3-carboxylate (1B) and ethyl 2-(2,4-difluorophenyl)-2,4,5,6,7,8-hexahydro-5,8-epoxycyclohepta[c]pyrazole-3-carboxylate (1B′). Mass Spectrum (LCMS. ESI pos): Calculated: for C₁₇H₁₆F₂N₂O₃: 335.1 (M+H), Measured: 335.1.

### C. 1-(2,4-difluorophenyl)-1,4,5,6,7,8-hexahydro-5,8-epoxycyclohepta[c]pyrazole-3-carboxylic acid (1C) and 2-(2,4-difluorophenyl)-2,4,5,6,7,8-hexahydro-5,8-epoxycyclohepta[c]pyrazole-3-carboxylic acid (1C′)

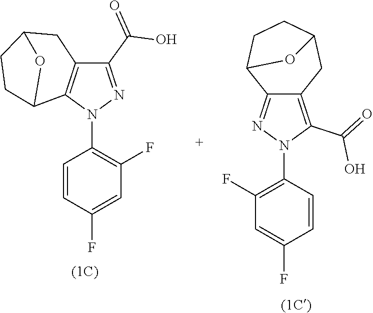 [SMILES: O=C(O)c1nn(-c2ccc(F)cc2F)c2c1CC1CCC2O1 ; O=C(O)c1c2c(nn1-c1ccc(F)cc1F)C1CCC(C2)O1]

To a solution of ethyl 1-(2,4-difluorophenyl)-1,4,5,6,7,8-hexahydro-5,8-epoxycyclohepta[c]pyrazole-3-carboxylate (1B) and ethyl 2-(2,4-difluorophenyl) -2,4,5,6,7,8-hexahydro-5,8-epoxycyclohepta[c]pyrazole-3-carboxylate (1B′) (450 mg, 1.5 mmol) in tetrahydrofuran, water and methanol (4:1:1, 12 mL) was added lithium hydroxide (215 mg, 8.9 mmol). The mixture was stirred at RT overnight. The organic solvents were removed under vacuum and the resultant was then treated with H₂O (4 mL). The pH of the solution was adjusted to 3 with aq. 2 M HCl. The white solid formed was collected and dried to yield the 1-(2,4-difluorophenyl)-1,4,5,6,7,8-hexahydro-5,8-epoxycyclohepta[c]pyrazole-3-carboxylic acid (1C) and 2-(2,4-difluorophenyl) -2,4,5,6,7,8-hexahydro-5,8-epoxycyclohepta[c]pyrazole-3-carboxylic acid (1C′). Mass Spectrum (LCMS. ESI pos.): Calculated: for C₁₅H₁₂F₂N₂O₃: 307.3 (M+H), Measured: 307.1.

### D. N-(tert-butyl)-1-(2,4-difluorophenyl)-1,4,5,6,7,8-hexahydro-5,8-epoxycyclohepta[c]pyrazole-3-carboxamide and N-(tert-butyl)-2-(2,4-difluorophenyl) -2,4,5,6,7,8-hexahydro-5,8-epoxycyclohepta[c]pyrazole-3-carboxamide

To a solution of a mixture of 1-(2,4-difluorophenyl)-1,4,5,6,7,8-hexahydro-5,8-epoxycyclohepta[c]pyrazole-3-carboxylic acid (1C) (100.0 mg, 0.327 mmol) and 2-(2,4-difluorophenyl)-2,4,5,6,7,8-hexahydro-5,8-epoxycyclohepta[c]pyrazole-3-carboxylic acid (1C′) (100.0 mg, 0.327 mmol) in DMF (5 mL) was added HATU (248 mg, 0.653 mmol) and DIEA (84.4 mg, 0.653 mmol), followed by 2-methylpropan-2-amine (47.8 mg, 0.653 mmol). The mixture was stirred at rt for overnight. The reaction was quenched with H₂O (100 mL). The resulting mixture was extracted with EtOAc (3×100 mL). The organic layers were combined, dried over Na₂SO₄, filtered and concentrated. The resulting residue was then purified by Prep-HPLC using H₂O (0.05% NH₄HCO₃) and CH₃CN to yield N-(tert-butyl)-1-(2,4-difluorophenyl)-1,4,5,6,7,8-hexahydro-5,8-epoxycyclohepta[c]pyrazole-3-carboxamide (Cpd. 1). ¹H-NMR (300 MHz, DMSO-d₆) δ (ppm): 7.66 (s, 1H), 7.39-7.58 (m, 2H), 7.17-7.23 (m, 1H), 5.11-5.12 (m, 1H), 4.70-4.74 (m, 1H), 3.09-3.16 (m, 1H), 2.45-2.51 (m, 1H), 1.94-2.16 (m, 2H), 1.87-1.94 (m, 1H), 1.69-1.71 (m, 1H), 1.26 (s, 9H). Mass Spectrum (LCMS. ESI pos.): Calculated: for C₁₉H₂₁F₂N₃O₂: 361.2 (M+H), Measured: 362.1 and N-(tert-butyl)-2-(2,4-difluorophenyl)-2,4,5,6,7,8-hexahydro-5,8-epoxycyclohepta[c]pyrazole-3-carboxamide (Cpd. 2). ¹H-NMR (300 MHz, DMSO-d₆) δ (ppm): 7.57-7.77 (m, 2H), 7.27-7.34 (m, 1H), 7.13 (s, 1H), 4.90 (s, 1H), 4.69-4.72 (m, 1H), 3.00-3.07 (m, 1H), 2.54-2.59 (m, 1H), 1.98-2.13 (m, 3H), 1.61-1.65 (m, 1H), 1.35 (s, 9H). Mass Spectrum (LCMS. ESI pos.): Calculated: for C₁₉H₂₁F₂N₃O₂; 361.2 (M+H), Measured: 362.1.

### Example 2

### N-(tert-butyl)-1-(pyrazin-2-yl)-1,4,5,6,7,8-hexahydro-5,8-epoxycyclohepta[c]pyrazole-3-carboxamide (Cpd. 3) and N-(tert-butyl)-2-(pyrazin-2-yl)-2,4,5,6,7,8-hexahydro-5,8-epoxycyclohepta[c]pyrazole-3-carboxamide (Cpd. 4)

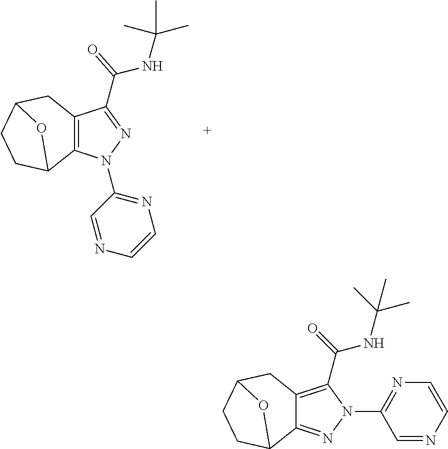 [SMILES: CC(C)(C)NC(=O)c1nn(-c2cnccn2)c2c1CC1CCC2O1 ; CC(C)(C)NC(=O)c1c2c(nn1-c1cnccn1)C1CCC(C2)O1]

### A. ethyl 1-(pyrazin-2-yl)-1,4,5,6,7,8-hexahydro-5,8-epoxycyclohepta[c]pyrazole-3-carboxylate (2 A) and ethyl 2-(pyrazin-2-yl)-2,4,5,6,7,8-nexahydro-5,8-epoxycyclohepta[c]pyrazole-3-carboxylate (2 A′)

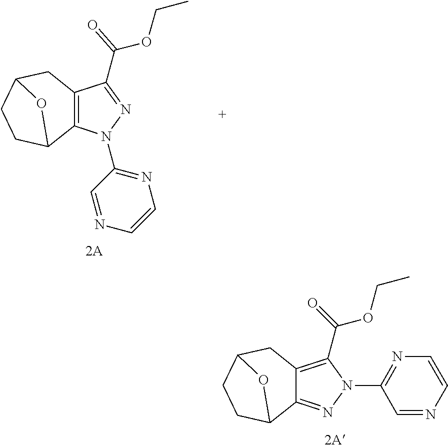 [SMILES: CCOC(=O)c1nn(-c2cnccn2)c2c1CC1CCC2O1 ; CCOC(=O)c1c2c(nn1-c1cnccn1)C1CCC(C2)O1]

The title compounds were prepared from ethyl 2-oxo-2-(2-oxo-8-oxabicyclo[3.2.1]octan-3-yl)acetate and 2-hydrazinylpyrazine using the procedure described in Example 1, step B. Mass Spectrum (LCMS. ESI pos.): Calculated: for C₁₅H₁₆N₄O₃: 301.1 (M+H), Measured: 301.0.

### B. 1-(pyrazin-2-yl)-1,4,5,6,7,8-hexahydro-5,8-epoxycyclohepta[c]pyrazole-3-carboxylic acid (2 B) and 2-(pyrazin-2-yl)-2,4,5,6,7,8-hexahydro-5,8-epoxycyclohepta[c]pyrazole-3-carboxylic acid (2 B′)

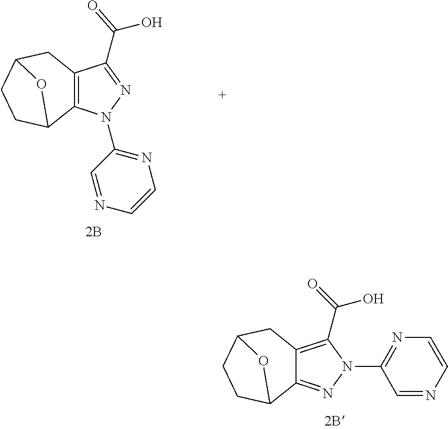 [SMILES: O=C(O)c1nn(-c2cnccn2)c2c1CC1CCC2O1 ; O=C(O)c1c2c(nn1-c1cnccn1)C1CCC(C2)O1]

The title compounds were prepared from ethyl 1-(pyrazin-2-y)-1,4,5,6,7,8-hexahydro-5,8-epoxycyclohepta[c]pyrazole-3-carboxylate (2 A) and ethyl 2-(pyrazin-2-yl)-2,4,5,6,7,8-hexahydro-5,8-epoxycyclohepta[c]pyrazole-3-carboxylate (2 A′) using the procedure described in Example 1, step C. Mass Spectrum (LCMS. ESI pos.): Calculated: for C₁₃H₁₂N₄O₃: 273.1 (M+H), Measured: 273.1.

### C. N-(tert-butyl)-1-(pyrazin-2-yl)-1,4,5,6,7,8-hexahydro-5,8-epoxycyclohepta[c]pyrazole-3-carboxamide (Cpd. 3) and N-(tert-butyl)-2-(pyrazin-2-yl) -2,4,5,6,7,8-hexahydro-5,8-epoxycyclohepta[c]pyrazole-3-carboxamide (Cpd. 4)

The title compounds were prepared from 1-(pyrazin-2-yl)-1,4,5,6,7,8-hexahydro -5,8-epoxycyclohepta[c]pyrazole-3-carboxylic acid (2B) and 2-(pyrazin-2-yl)-2,4,5,6,7,8-hexahydro-5,8-epoxycyclohepta[c]pyrazole-3-carboxylic acid (2 B′) using the procedure described in Example 1, step D. N-(tert-butyl)-1-(pyrazin-2-yl)-1,4,5,6,7,8-hexahydro -5,8-epoxycyclohepta[c]pyrazole-3-carboxamide (Cpd. 3), ¹H-NMR (300 MHz, DMSO-d₆) δ (ppm): 8.98 (s, 1H), 8.56-8.57 (m, 1H), 8.43-8.45 (m, 1H), 8.13 (s, 1H), 5.17-5.20 (m, 1H), 4.72-4.75 (m, 1H), 2.96-3.03 (m, 1H), 2.40-2.45 (m, 1H), 2.08-2.18 (m, 2H), 1.90-1.96 (m, 1H), 1.66-1.68 (m, 1H), 1.32 (s, 9H). Mass Spectrum (LCMS. ESI pos,): Calculated: for C₁₇H₂₁N₅O₂: 328.2 (M+H), Measured: 328.2 and N-(tert-butyl)-2-(pyrazin-2-yl)-2,4,5,6,7,8-hexahydro-5,8-epoxycyclohepta[c]pyrazole-3-carboxamide (Cpd. 4), ¹H-NMR (300 MHz, DMSO-d₆) δ (ppm): 9.45 (s, 1H), 8.62-8.63 (m, 1H), 8.55-8.56 (m, 1H), 7.50 (s, 1H), 5.79 (s, 1H), 4.70-4.74 (m, 1H), 3.01-3.08 (m, 1H), 2.51-2.61 (m, 1H), 2.14 (s, 3H), 1.50-1.65 (m, 1H), 1.40 (s, 9H). Mass Spectrum (LCMS. ESI pos.): Calculated: for C₁₇H₂₁N₅O₂: 328.2 (M+H), Measured: 328.2.

Following the procedures described in Example 1 and substituting suitably selected starting materials, reagents and conditions, as it would be readily apparent to those skilled in the art, the following representative compounds of the present invention were prepared.

| | |
|---|---|
| Example 3 | 2-(2,4-difluorophenyl)-N-((2R,3as,5S,6as)-hexahydro-2,5- |
| | methanopentalen-3a(1H)-yl)-2,4,5,6,7,8-hexahydro-5,8- |
| | epoxycyclohepta[c]pyrazole-3-carboxamide (Cpd. 5).¹H-NMR (300 |
| | MHz, CD₃OD) δ (ppm): 7.20-7.62 (m, 1H), 7.10-7.19 (m, 2H), 4.89- |
| | 5.18 (m, 1H), 3.98-4.82 (m, 1H), 3.17-3.34 (m, 1H), 2.56-2.61 (m, |
| | 1H), 1.90-1.91 (m, 4H), 1.82-2.03 (m, 9H), 1.58-1.68 (m, 4H). |
| | Mass Spectrum (LCMS. ESI pos.): Calculated: for C₂₄H₂₅F₂N₃O₂: |
| | 426.2 (M + H), Measured: 426.2 and |
| | 1-(2,4-difluorophenyl)-N-((2R,3as,5S,6as)-hexahydro-2,5- |
| | methanopentalen-3a(1H)-yl)-1,4,5,6,7,8-hexahydro-5,8- |
| | epoxycyclohepta[c]pyrazole-3-carboxamide (Cpd. 41). ¹H-NMR (300 |
| | MHz, CD₃OD) δ (ppm): 7.20-7.63 (m, 1H), 7.30-7.41 (m, 1H), 7.18- |
| | 7.28 (m, 1H), 4.89-5.01 (m, 1H), 4.75-4.85 (m, 1H), 3.12-3.25 |
| | (m, 1H), 2.71-2.80 (m, 1H), 2.56-2.69 (m, 1H), 2.01-2.49 (m, |
| | 12H), 1.58-1.86 (m, 5H). Mass Spectrum (LCMS. ESI pos.): |
| | Calculated: for C₂₄H₂₅F₂N₃O₂: 426.2 (M + H), Measured: 426.2. |
| Example 4 | N-(((3r,5r,7r)-adamantan-1-yl)methyl)-2-(2,4-difluorophenyl)- |
| | 2,4,5,6,7,8-hexahydro-5,8-epoxycyclohepta[c]pyrazole-3-carboxamide |
| | (Cpd. 6). ¹H-NMR (300 MHz, DMSO-d₆) δ (ppm): 8.05-8.10 (m, 1H), |
| | 7.59-7.51 (m, 1H), 7.41-7.45 (m, 1H), 7.17-7.39 (m, 1H), 5.12- |
| | 5.14 (m, 1H), 4.77-4.78 (m, 1H), 3.14-3.21 (m, 1H), 2.83-2.86 (m, |
| | 2H), 2.51-2.61 (m, 1H), 2.05-2.18 (m, 2H), 1.90-1.91 (m, 4H), 1.51- |
| | 1.71 (m, 7H), 1.38-1.42 (m, 6H). Mass Spectrum (LCMS. ESI pos.): |
| | Calculated: for C₂₆H₂₉F₂N₃O₂: 454.2 (M + H), Measured: 454.2. and |
| | N-(((3r,5r,7r)-adamantan-1-yl)methyl)-1-(2,4-difluorophenyl)- |
| | 1,4,5,6,7,8-hexahydro-5,8-epoxycyclohepta[c]pyrazole-3-carboxamide |
| | (Cpd. 42). ¹H-NMR (400 MHz, DMSO-d₆) δ (ppm): 7.71-7.83 (m, |
| | 2H), 7.59-7.64 (m, 1H), 7.29-7.34 (m, 1H), 4.90-4.91 (m, 1H), 4.70- |
| | 4.72 (m, 1H), 2.87-3.64 (m, 3H), 2.55-2.59 (m, 1H), 2.09-2.14 (m, |
| | 1H), 1.97-2.05 (m, 2H), 1.91 (s, 3H), 1.45-1.66 (m, 7H), 1.45 (s, |
| | 6H). Mass Spectrum (LCMS. ESI pos.): Calculated: for C₂₆H₂₉F₂N₃O₂: |
| | 454.2 (M + H), Measured: 454.2. |
| Example 5 | (5S,8R)-N-((S)-1-hydroxy-3,3-dimethylbutan-2-yl)-1-(pyrazin-2- |
| | yl)-1,4,5,6,7,8-hexahydro-5,8-epoxycyclohepta[c]pyrazole-3- |
| | carboxamide (Cpd. 7) ¹H-NMR (400 MHz, CD₃OD) δ (ppm): 9.40 (d, |
| | J = 1.2 Hz, 1H), 8.56 (d, J = 1.2 Hz, 1H), 8.50-8.51 (m, 1H), 5.93- |
| | 5.94 (m, 1H), 4.79-4.82 (m, 1H), 3.97-4.00 (m, 1H), 3.84-3.88 |
| | (m, 1H), 3.70-3.73 (m, 1H), 3.20-3.25 (m, 1H), 2.66-2.70 (m, 1H), |
| | 2.25-2.28 (m, 3H), 1.70-1.71 (m, 1H), 1.00-1.07 (m, 9H). Mass |
| | Spectrum (LCMS. ESI pos.): Calculated: for C₁₉H₂₅N₅O₃: 372.1 |
| | Measured: 372.1 and |
| | (5R,8S)-N-((S)-1-hydroxy-3,3-dimethylbutan-2-yl)-1-(pyrazin-2- |
| | yl)-1,4,5,6,7,8-hexahydro-5,8-epoxycyclohepta[c]pyrazole-3- |
| | carboxamide (Cpd. 8). ¹H-NMR (400 MHz, CD₃OD) δ (ppm): 9.40 (d, |
| | J = 1.2 Hz, 1H), 8.56 (d, J = 1.2 Hz, 1H), 8.50-8.51 (m, 1H), 5.90- |
| | 5.92 (m, 1H), 4.79-4.82 (m, 1H), 3.97-4.00 (m, 1H), 3.85-3.89 |
| | (m, 1H), 3.70-3.75 (m, 1H), 3.15-3.33 (m, 1H), 2.70-2.74 (m, 1H), |
| | 2.25-2.28 (m, 3H), 1.70-1.76 (m, 1H), 1.01-1.06 (m, 9 H). Mass |
| | Spectrum (LCMS. ESI pos.): Calculated: For C₁₉H₂₅N₅O₃ (M + H): |
| | 372.1 Measured: 372.1 |
| Example 6 | 1-(pyrazin-2-yl)-N-(1-(trifluoromethyl)cyclopropyl)-1,4,5,6,7,8- |
| | hexahydro-5,8-epoxycyclohepta[c]pyrazole-3-carboxamide (Cpd. 9). |
| | ¹H-NMR (300 MHz, DMSO-d₆) δ (ppm): 9.48 (d, J = 1.2 Hz, 1H), 9.09- |
| | 9.21 (m, 1H), 8.64-8.65 (m, 1H), 8.57 (dd, J₁ = 1.2 Hz, J₂ = 1.5 Hz, |
| | 1H), 5.78-5.89 (m, 1H), 4.74 (t, J = 4.8 Hz, 1H), 3.01-3.09 (m, 1H), |
| | 2.59-2.63 (m, 1H), 2.07-2.21 (m, 3H), 1.57-1.65 (m, 1H), 1.31- |
| | 1.36 (m, 2H), 1.11-1.21 (m, 2H). Mass Spectrum (LCMS. ESI pos.): |
| | Calculated: For C₁₇H₁₆F₃N₅O₂ (M + H): 380.1 Measured: 380.1 |
| Example 7 | 1-(pyrazin-2-yl)-N-(1-(trifluoromethyl)cyclobutyl)-1,4,5,6,7,8- |
| | hexahydro-5,8-epoxycyclohepta[c]pyrazole-3-carboxamide (Cpd. 10). |
| | ¹H-NMR (300 MHz, DMSO-d₆) δ (ppm): 9.52 (d, J = 1.5 Hz, 1H), 8.75 |
| | (s, 1H), 8.64-8.66 (m, 1H), 8.57 (dd, J₁ = 1.5 Hz, J₂ = 1.5 Hz, 1H), |
| | 5.80-5.87 (m, 1H), 4.74 (m, 1H), 3.02 (dd, J₁ = 4.8 Hz, J₂ = 4.8 Hz, |
| | 1H), 2.60-2.69 (m, 4H), 2.50-2.52 (m, 1H), 2.10-2.18 (m, 3H), 1.98- |
| | 2.03 (m, 2H), 1.64-1.66 (m, 1H). Mass Spectrum (LCMS. ESI pos.): |
| | Calculated: For C₁₈H₁₈F₃N₅O₂ (M + H): 394.1 Measured: 394.1 |
| Example 8 | N-(1-hydroxy-2-methylpropan-2-yl)-1-(pyrazin-2-yl)-1,4,5,6,7,8- |
| | hexahydro-5,8-epoxycyclohepta[c]pyrazole-3-carboxamide (Cpd. 11). |
| | ¹H-NMR (400 MHz, CD₃OD) δ (ppm): 9.35 (s, 1 H), 8.55 (d, J = 2.8 |
| | Hz, 1 H), 8.49-8.50 (m, 1 H), 5.90 (d, J = 2.4 Hz, 1 H), 4.80 (t, J = 4.8 |
| | Hz, 1 H), 3.66 (s, 2 H), 3.16-3.21 (m, 1 H), 2.66-2.70 (m, 1 H), 2.23- |
| | 2.29 (m, 3 H), 1.69-1.73 (m, 1 H), 1.44 (s, 6 H). Mass Spectrum |
| | (LCMS. ESI pos.): Calculated: For C₁₇H₂₁N₅O₃(M + H): 344.2 |
| | Measured: 344.1 |
| Example 9 | N-(bicyclo[1.1.1]pentan-1-yl)-1-(pyrazin-2-yl)-1,4,5,6,7,8- |
| | hexahydro-5,8-epoxycyclohepta[c]pyrazole-3-carboxamide (Cpd. 12). |
| | ¹H-NMR (400 MHz, DMSO-d₆) δ (ppm): 9.42 (d, J = 1.6 Hz, 1H), 8.80- |
| | 8.89 (m, 1H), 8.62 (d, J = 2.4 Hz, 1H), 8.56 (dd, J₁ = 1.2 Hz, J₂ = 1.6 |
| | Hz, 1H), 5.76-5.83 (m, 1H), 4.71-4.74 (m, 1H), 3.05 (dd, J₁ = 4.8 |
| | Hz, J₂ = 5.2 Hz, 1H), 2.55-2.60 (m, 1H), 2.43-2.47 (m, 1H), 2.12- |
| | 2.16 (m, 3H), 2.03-2.11 (m, 6H), 1.60-1.63 (m, 1H). Mass Spectrum |
| | (LCMS. ESI pos.): Calculated: For C₁₈H₁₉N₅O₂(M + H): 338.2 |
| | Measured: 338.1. |
| Example 10 | N-((2R,3as,5S,6as)-hexahydro-2,5-methanopentalen-3a(1H)- |
| | yl)-1-(pyrazin-2-yl)-1,4,5,6,7,8-hexahydro-5,8- |
| | epoxycyclohepta[c]pyrazole-3-carboxamide (Cpd. 13). ¹H-NMR (400 |
| | MHz, CD₃OD) δ (ppm): 9.39 (d, J = 1.2 Hz, 1 H), 8.54-8.55 (m, 1 H), |
| | 8.49 (t, J = 1.2 Hz, 1 H), 5.91 (s, 1 H), 4.80 (t, J = 5.2 Hz, 1 H), 3.17- |
| | 3.22 (m, 1 H), 2.59-2.70 (m, 2 H), 2.28-2.32 (m, 7 H), 2.05-2.14 |
| | (m, 4 H), 1.61-1.74 (m, 5 H). Mass Spectrum (LCMS. ESI pos.): |
| | Calculated: For C₂₂H₂₅N₅O₂(M + H): 392.5 Measured: 392.1. |
| Example 11 | N-(bicyclo[2.2.2]octan-1-yl)-1-(pyrazin-2-yl)-1,4,5,6,7,8- |
| | hexahydro-5,8-epoxycyclohepta[c]pyrazole-3-carboxamide (Cpd. |
| | 14).¹H-NMR (300 MHz, CD₃OD) δ (ppm): 9.34 (s, 1H), 8.56 (d, J = 2.7 |
| | Hz, 1H), 8.49-8.50 (m, 1H), 5.90 (d, J = 3.0 Hz, 1H), 4.79 (t, J = 4.8 |
| | Hz, 1H), 3.13-3.19 (m, 1H), 2.63-2.68 (m, 1H), 2.20-2.32 (m, 3H), |
| | 2.00-2.05 (m, 6H), 1.70-1.79 (m, 7H), 1.59-1.66 (m, 1H). Mass |
| | Spectrum (LCMS. ESI pos.): Calculated: For C₂₁H₂₅N₅O₂(M + H): 380.2 |
| | Measured: 380.2. |

### Example 12

### 3-((5S, 8R)-3-(((S)-1-hydroxy-3,3-dimethylbutan-2-yl)carbamoyl)-5,6,7,8-tetrahydro-5,8-epoxycyclohepta[c]pyrazol-1(4H)-yl)pyrazine 1-oxide (Cpd. 15) and 3-((5R,8S)-3-(((S) -1-hydroxy-3,3-dimethylbutan-2-yl)carbamoyl)-5,6,7,8-tetrahydro-5,8-epoxycyclohepta[c]pyrazol-1(4H)-yl)pyrazine 1-oxide (Cpd. 16)

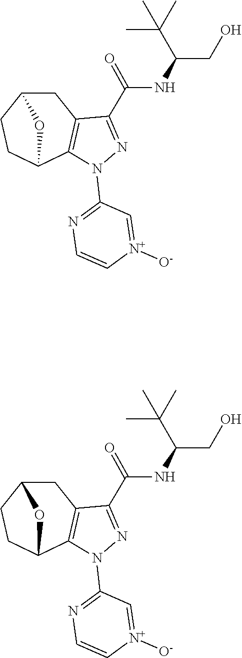 [SMILES: CC(C)(C)[C@@H](CO)NC(=O)c1nn(-c2c[n+]([O-])ccn2)c2c1C[C@@H]1CC[C@H]2O1 ; CC(C)(C)[C@@H](CO)NC(=O)c1nn(-c2c[n+]([O-])ccn2)c2c1C[C@H]1CC[C@@H]2O1]

To a solution of N-((S)-1-hydroxy-3,3-dimethylbutan-2-yl)-1-(pyrazin-2-yl) -1,4,5,6,7,8-hexahydro-5,8-epoxycyclohepta[c]pyrazole-3-carboxamide (compound 7, 50.0 mg, 0.404 mmol,) in dichloromethane (20 mL) was added 3-chlorobenzoperoxoic acid (288 mg, 1.62 mmol). The resulting mixture was stirred at 40° C. overnight. After cooling to r.t., the reaction mixture was concentrated and the resulting residue was purified by reversed phase Prep-HPLC using H₂O (0.05% NH₄HCO₃) and CH₃CN to yield 3-((5S,8R)-3-(((S)-1-hydroxy-3,3-dimethylbutan-2-yl)carbamoyl)-5,6,7,8-tetrahydro-5,8-epoxycyclohepta[c]pyrazol-1(4H)-yl)pyrazine 1-oxide (Cpd. 15). ¹H-NMR (300 MHz, DMSO-d₆) δ (ppm): 9.20 (d, 1H), 8.45-8.47 (m, 1H), 8.28 (dd, 1H), 7.78 (dd, 1H), 5.78-5.87 (m, 1H), 4.78-4.77 (m, 1H), 4.48-4.53 (m, 1H), 3.79-3.86 (m, 1H), 3.65-3.72 (m, 1H), 3.46-3.54 (m, 1H), 3.04 (dd, 1H), 2.50-2.51 (m, 1H), 2.07-2.17 (m, 3H), 1.59-1.62 (m, 1H), 0.90-0.99 (m, 9H). Mass Spectrum (LCMS. ESI pos.): Calculated: For C₁₉H₂₅N₅O₄ (M+H): 388.19, Measured: 388.2, and 3-((5R,8S)-3-(((S)-1-hydroxy-3,3-dimethylbutan-2-yl)carbamoyl)-5,6,7,8-tetrahydro-5,8-epoxycyclohepta[c]pyrazol-1(4H)-yl)pyrazine 1-oxide (Cpd. 16). ¹H-NMR (300 MHz, DMSO-d₆) δ (ppm): 9.20 (d, 1H), 8.45-8.47 (m, 1H), 8.29 (dd, 1H), 7.80 (dd, 1H), 5.79-5.80 (m, 1H), 4.73-4.74 (m, 1H), 4.50-4.55 (m, 1H), 3.79-3.86 (m, 1H), 3.65-3.72 (m, 1H), 3.47-3.55 (m, 1H), 3.00 (dd, 1H), 2.58-2.64 (m, 1H), 2.15-2.20 (m, 3H), 1.58-1.69 (m, 1H), 0.87-1.97 (m, 9H). Mass Spectrum (LCMS. ESI pos.): Calculated: For C₁₉H₂₅N₅O₄ (M+H): 388.19, Measured: 388.2.

Following the procedures described in Example 12 above, and substituting suitably selected reagents, starting materials and conditions as it would be readily apparent to those skilled in the art, the following representative compounds of the present invention were prepared.

| | |
|---|---|
| Example 13 | 3-(3-((1-(trifluoromethyl)cyclopropyl)carbamoyl)-5,6,7,8- |
| | tetrahydro-5,8-epoxycyclohepta[c]pyrazol-1(4H)-yl)pyrazine 1-oxide |
| | (Cpd. 17). ¹H-NMR (300 MHz, DMSO-d₆) δ (ppm): 9.20-9.26 (m, |
| | 2H), 8.44-8.46 (m, 1H), 8.29-8.31 (m, 1H), 5.78-5.86 (m, 1H), |
| | 4.68-4.76 (m, 1H), 2.99-3.07 (m, 1H), 2.54-2.62 (m, 1H), 2.08- |
| | 2.28 (m, 3H), 1.49-1.63 (m, 1H), 1.28-1.37 (m, 2H), 1.11-1.20 (m, |
| | 2H). Mass Spectrum (LCMS. ESI pos.): Calculated: For |
| | C₁₇H₁₆F₃N₅O₃ (M + H): 396.1, Measured: 396.2. |
| Example 14 | 3-(3-((1-(trifluoromethyl)cyclobutyl)carbamoyl)-5,6,7,8- |
| | tetrahydro-5,8-epoxycyclohepta[c]pyrazol-1(4H)-yl)pyrazine 1-oxide |
| | (Cpd. 18). ¹H-NMR (300 MHz, DMSO-d₆) δ (ppm): 9.25 (dd, 1H), |
| | 8.79-8.86 (m, 1H), 8.45 (dd, 1H), 8.30 (dd, 1H), 5.76-5.87 (m, |
| | 1H), 4.72-4.45 (m, 1H), 2.98-3.06 (m, 1H), 2.52-2.71 (m, 4H), |
| | 2.45-2.50 (m, 1H), 2.07-2.19 (m, 3H), 1.89-1.98 (m, 2H), 1.45- |
| | 1.72 (m, 1H). Mass Spectrum (LCMS. ESI pos.): Calculated: For |
| | C₁₈H₁₈F₃N₅O₃ (M + H): 410.14, Measured: 410.1. |
| Example 15 | 3-(3-((1-hydroxy-2-methylpropan-2-yl)carbamoyl)-5,6,7,8- |
| | tetrahydro-5,8-epoxycyclohepta[c]pyrazol-1(4H)-yl)pyrazine 1-oxide |
| | (Cpd. 19). ¹H-NMR (300 MHz, DMSO-d₆) δ (ppm): 9.07 (d, 1H), 8.44- |
| | 8.47 (m, 1H), 8.30 (dd, 1H), 7.45-7.59 (m, 1H), 5.73-5.84 (m, |
| | 1H), 5.02-5.06 (m, 1H), 4.72-4.74 (m, 1H), 3.47-3.49 (m, 2H), |
| | 3.01 (dd, 1H), 2.56-2.64 (m, 1H), 2.08-2.14 (m, 3H), 1.59-1.63 (m, |
| | 1H), 1.32 (s, 6H). Mass Spectrum (LCMS. ESI pos.): Calculated: For |
| | C₁₇H₂₁N₅O₄ (M⁺ + H): 360.2, Measured: 360.1. |
| Example 16 | 3-(3-(bicyclo[1.1.1]pentan-1-ylcarbamoyl)-5,6,7,8-tetrahydro- |
| | 5,8-epoxycyclohepta[c]pyrazol-1(4H)-yl)pyrazine 1-oxide (Cpd. 20). |
| | ¹H-NMR (400 MHz, DMSO-d₆) δ (ppm): 9.10 (d, 1H), 8.90-8.99 (m, |
| | 1 H), 8.43-8.45 (m, 1H), 8.28 (dd, 1H), 5.74-5.85 (m, 1H), 4.70- |
| | 4.73 (m, 1H), 3.03 (dd, 1H), 2.54-2.58 (m, 1H), 2.45-2.52 (m, 1H), |
| | 2.15-2.16 (m, 3H), 2.09-2.13 (m, 6H), 1.57-1.61 (m, 1H). Mass |
| | Spectrum (LCMS. ESI pos.): Calculated: For C₁₈H₁₉N₅O₃ (M⁺ + H): |
| | 354.1, Measured: 354.1. |
| Example 17 | 3-(3-(((2R,3as,5S,6as)-hexahydro-2,5-methanopentalen- |
| | 3a(1H)-yl)carbamoyl)-5,6,7,8-tetrahydro-5,8- |
| | epoxycyclohepta[c]pyrazol-1(4H)-yl)pyrazine 1-oxide (Cpd. 21). ¹H- |
| | NMR (300 MHz, DMSO-d₆) δ (ppm): 9.24 (d, 1H), 8.44-8.45 (m, |
| | 1H), 8.28-8.30 (m, 1H), 8.20-8.27 (m, 1H), 5.74-5.86 (m, 1H); |
| | 4.66-4.77 (m, 1H), 2.99-3.07 (m, 1H), 2.56-2.67 (m, 1H), 2.18- |
| | 2.29 (m, 2H), 2.07-2.19 (m, 5H), 1.92-2.13 (m, 4H), 1.53-1.63 |
| | (m, 5H), 1.17-2.28 (m, 1H). Mass Spectrum (LCMS. ESI pos.): |
| | Calculated: For C₂₂H₂₅N₅O₃ (M⁺ + H): 408.2, Measured: 408.2. |
| Example 18 | 3-(3-(bicyclo[2.2.2]octan-1-ylcarbamoyl)-5,6,7,8-tetrahydro- |
| | 5,8-epoxycyclohepta[c]pyrazol-1(4H)-yl)pyrazine 1-oxide (Cpd. 22). |
| | ¹H-NMR (400 MHz, DMSO-d₆) δ (ppm): 9.17-9.26 (m, 1H), 8.42 (d, |
| | 1H), 8.27 (t, 1H), 7.40-7.49 (m, 1H), 5.77-5.86 (m, 1H), 4.63- |
| | 4.75 (m, 1H), 3.03 (d, 1H), 2.17-2.16 (m, 3H), 1.82-1.93 (m, 6H), |
| | 1.57-1.66 (m, 8H). Mass Spectrum (LCMS. ESI pos.): Calculated: |
| | For C₂₁H₂₅N₅O₃ (M⁺ + H): 396.2, Measured: 396.2. |
| Example 19 | 3-(3-(5-(tert-butyl)-1,2,4-oxadiazol-3-yl)-5,6,7,8-tetrahydro-5,8- |
| | epoxycyclohepta[c]pyrazol-1(4H)-yl)pyrazine 1-oxide (Cpd. 23). ¹H- |
| | NMR (300 MHz, DMSO-d₆) δ (ppm): 8.62 (s, 1H), 8.48 (d, J = 4.0 |
| | Hz, 1H), 8.32-8.34 (m, 1H), 5.81 (d, J = 4.4 Hz, 1H), 4.80 (d, J = 4.8 |
| | Hz, 1H), 3.08-3.13 (m, 1H), 2.58 (d, J = 16.0 Hz, 1H), 2.16-2.22 |
| | (m, 3H), 1.43-1.45 (m, 1H), 1.42 (s, 9H). Mass Spectrum (LCMS. |
| | ESI pos.): Calculated: For C₁₈H₂₀N₆O₃ (M⁺ + H): 369.1, Measured: |
| | 369.2. |
| Example 20 | 3-(3-(5-(tert-butyl)thiazol-2-yl)-5,6,7,8-tetrahydro-5,8- |
| | epoxycyclohepta[c]pyrazol-1(4H)-yl)pyrazine 1-oxide (Cpd. 24). ¹H- |
| | NMR (400 MHz, DMSO-d₆) δ (ppm): 8.61 (s, 1H), 8.45-8.46 (m, |
| | 1H), 8.28-8.29 (m, 1H), 7.71 (s, 1H), 5.81-5.83 (m, 1H), 4.78- |
| | 4.79 (m, 1H), 3.11-3.16 (m, 1H), 2.64-2.68 (m, 1H), 2.15-2.21 (m, |
| | 3H), 1.60-1.63 (m, 1H), 1.40 (s, 9H). Mass Spectrum (LCMS. ESI |
| | pos.): Calculated: For C₁₉H₂₁N₅O₂S (M⁺ + H): 384.1, Measured: |
| | 384.1. |
| Example 21 | 3-(3-(4-(tert-butyl)thiazol-2-yl)-5,6,7,8-tetrahydro-5,8- |
| | epoxycyclohepta[c]pyrazol-1(4H)-yl)pyrazine 1-oxide (Cpd. 44). ¹H- |
| | NMR (300 MHz, DMSO-d₆) δ (ppm): 8.62 (s, 1H), 8.46-8.47 (m, |
| | 1H), 8.28-8.30 (m, 1H), 7.37 (s, 1H), 5.82-5.83 (m, 1H), 4.81- |
| | 4.85 (m, 1H), 3.14-3.22 (m, 1H), 2.68-2.73 (m, 1H), 2.15-2.22 (m, |
| | 3H), 1.66-1.69 (m, 1H), 1.34 (s, 9H). Mass Spectrum (LCMS. ESI |
| | pos.): Calculated: For C₁₉H₂₁N₅O₂S (M⁺ + H): 384.1, Measured: |
| | 384.1. |
| Example 22 | 3-(3-(3-(tert-pentyl)-1,2,4-oxadiazol-5-yl)-5,6,7,8-tetrahydro- |
| | 5,8-epoxycyclohepta[c]pyrazol-1(4H)-y)pyrazine 1-oxide (Cpd. 25). |
| | ¹H-NMR (300 MHz, DMSO-d₆) δ (ppm): 8.68 (dd, 1H), 8.52 (dd, 1H), |
| | 8.38 (dd, 1H), 5.82-5.84 (m, 1H), 4.83-4.85 (m, 1H), 3.13-3.20 |
| | (m, 1H), 2.64-2.71 (m, 1H), 2.16-2.28 (m, 3H), 1.65-1.77 (m, 3H), |
| | 1.31-1.37 (m, 6H), 1.71-1.82 (m, 3H). (LCMS. ESI pos.): Mass |
| | Spectrum Calculated: For C₁₉H₂₁N₅O₂S (M⁺ + H): 383.2, Measured: |
| | 383.2. |
| Example 23 | 3-(3-(3-(tert-butyl)-1,2,4-oxadiazol-5-yl)-5,6,7,8-tetrahydro-5,8- |
| | epoxycyclohepta[c]pyrazol-1(4H)-yl)pyrazine 1-oxide (Cpd. 26). ¹H- |
| | NMR (300 MHz, DMSO-d₆) δ (ppm): 8.68 (dd, 1H), 8.50 (dd, 1H), |
| | 8.38 (dd, 1H), 8.52-8.54 (m, 1H), 4.83-4.85 (m, 1H), 3.13-3.21(m, |
| | 1H), 2.49-2.71 (m, 1H), 2.14-2.28 (m, 3H), 1.61-1.78 (m, 1H), |
| | 1.31-1.42(m, 9H). Mass Spectrum (LCMS. ESI pos.): Calculated: |
| | For C₁₈H₂₀N₆O₃ (M⁺ + H): 369.2, Measured: 369.2. |
| Example 24 | 3-(3-(5-(tert-butyl)-1,3,4-thiadiazol-2-yl)-5,6,7,8-tetrahydro-5,8- |
| | epoxycyclohepta[c]pyrazol-1(4H)-yl)pyrazine 1-oxide (Cpd. 27). ¹H- |
| | NMR (400 MHz, DMSO-d₆) δ (ppm): 8.65 (s, 1H), 8.47-8.48 (m, |
| | 1H), 8.31-8.32 (m, 1H), 5.83-5.84 (m, 1H), 4.82-4.83 (m, 1H), |
| | 3.15-3.20 (m, 1H), 2.68-2.72 (m, 1H), 2.20-2.35 (m, 3H), 1.60- |
| | 1.63 (m, 1H), 1.47(s, 9H). (LCMS. ESI pos.): Calculated: For |
| | C₁₈H₂₀N₆O₂S (M⁺ + H): 385.1, Measured: 385.1 and |
| | 3-(3-(5-(tert-butyl)-1-oxido-1,3,4-thiadiazol-2-yl)-5,6,7,8- |
| | tetrahydro-5,8-epoxycyclohepta[c]pyrazol-1(4H)-yl)pyrazine 1-oxide |
| | (Cpd. 28). ¹H-NMR (400 MHz, DMSO-d₆) δ (ppm): 8.71 (s, 1H), 8.48- |
| | 8.49 (m, 1H), 8.33-8.35 (m, 1H), 5.82-5.83 (m, 1H), 4.81-4.82 |
| | (m, 1H), 3.08-3.12 (m, 1H), 2.60-2.67 (m, 1H), 2.15-2.23 (m, 3H), |
| | 1.63-1.67 (m, 1H), 1.47(s, 9H). Mass Spectrum (LCMS. ESI pos.): |
| | Calculated: For C₁₈H₂₀N₆O₃S (M⁺ + H): 401.1, Measured: 401.1 |
| Example 25 | 3-(3-(5-(tert-butyl)-1,3,4-oxadiazol-2-yl)-5,6,7,8-tetrahydro-5,8- |
| | epoxycyclohepta[c]pyrazol-1(4H)-yl)pyrazine 1-oxide (Cpd. 29). ¹H- |
| | NMR (400 MHz, DMSO-d₆) δ (ppm): 8.65 (s, 1H), 8.49-8.50 (m, |
| | 1H), 8.33-8.35 (m, 1H), 5.82-5.83 (m, 1H), 4.82-4.83 (m, 1H), |
| | 3.11-3.16 (m, 1H), 2.62-2.76 (m, 1H), 2.16-2.22 (m, 3H), 1.60- |
| | 1.67(m, 1H), 1.43(s, 9H). Mass Spectrum (LCMS. ESI pos.): |
| | Calculated: For C₁₈H₂₀N₆O₃ (M⁺ + H): 369.2, Measured: 396.1 |
| Example 26 | 3-(3-(tert-butylcarbamoyl)-5,6,7,8-tetrahydro-5,8- |
| | epoxycyclohepta[c]pyrazol-2(4H)-yl)pyrazine 1-oxide (Cpd. 30). ¹H- |
| | NMR (300 MHz, CD₃OD) δ (ppm): 9.05 (s, 1H), 8.42-8.43 (m, 1H), |
| | 8.19-8.21 (m, 1H), 5.90-5.91 (m, 1H), 4.79-4.81 (m, 1H), 3.14- |
| | 3.21 (m, 1H), 2.63-2.69 (m, 1H), 2.23-2.27 (m, 3H), 1.65-1.75 (m, |
| | 1H), 1.46 (s, 9H). Mass Spectrum (LCMS. ESI pos.): Calculated: For |
| | C₁₇H₂₁N₅O₃ (M⁺ + H): 344.2, Measured: 344.1 |
| Example 27 | 3-(3-(tert-butylcarbamoyl)-5,6,7,8-tetrahydro-5,8- |
| | epoxycyclohepta[c]pyrazol-1(4H)-yl)pyrazine 1-oxide (Cpd. 31). ¹H- |
| | NMR (300 MHz, CD₃OD) δ (ppm): 8.70-8.71 (m, 1H), 8.31-8.33 |
| | (m, 1H), 8.15-8.17 (m, 1H), 5.18-5.20 (m, 1H), 4.78-4.82 (m, 1H), |
| | 3.03-3.09 (m, 1H), 2.47-2.52 (m, 1H), 2.20-2.27 (m, 2H), 2.00- |
| | 2.07 (m, 1H), 1.70-1.85 (m, 1H), 1.44 (s, 9H). Mass Spectrum |
| | (LCMS. ESI pos.): Calculated: For C₁₇H₂₁N₅O₃ (M⁺ + H): 344.2, |
| | Measured: 344.1 |

### Example 28

### 3-(5-(tert-butyl)-1,2,4-oxadiazol-3-yl)-1-(pyrazin-2-yl)-1,4,5,6,7,8-hexahydro-5,8-epoxycyclohepta[c]pyrazole (Cpd. 32)

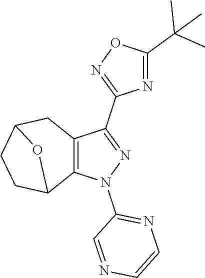 [SMILES: CC(C)(C)c1nc(-c2nn(-c3cnccn3)c3c2CC2CCC3O2)no1]

### A. 1-(pyrazin-2-yl)-1,4,5,6,7,8-hexahydro-5,8-epoxycyclohepta[c]pyrazole-3-carbonitrile (28 A)

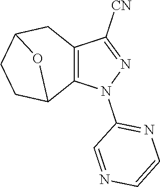 [SMILES: N#Cc1nn(-c2cnccn2)c2c1CC1CCC2O1]

To a stirred solution of 1-(pyrazin-2-yl)-1,4,5,6,7,8-hexahydro-5,8-epoxycyclohepta[c]pyrazole-3-carboxamide (37 A) (200.0 mg, 0.737 mmol) in dichloromethane (2 mL) and triethylamine (486 mg, 1.84 mmol) at 0° C., trifluoromethanesulfonic anhydride (232.0 mg, 1.11 mmol) was added. The resulting mixture was stirred at rt overnight. The reaction was then quenched with NaHCO₃ (10 mL) and extracted with ethyl acetate (3×50 mL). The organic layers were combined, dried over Na₂SO₄, filtered and concentrated. The resulting residue was purified by silica gel chromatography (Ethyl acetate/petroleum ether) to yield the title compound (28 A). Mass Spectrum (LCMS. ESI pos.): Calculated: For C₁₃H₁₁N₅O (M⁺+H): 254.1, Measured: 254.1.

### B. N-hydroxy-1-(pyrazin-2-yl)-1,4,5,6,7,8-hexahydro-5,8-epoxycyclohepta[c]pyrazole-3-carboximidam ide (28 B)

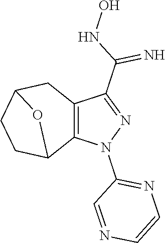 [SMILES: N=C(NO)c1nn(-c2cnccn2)c2c1CC1CCC2O1]

To a solution of 1-(pyrazin-2-yl)-1,4,5,6,7,8-hexahydro-5,8-epoxycyclohepta[c]pyrazole-3-carbonitrile (28 A) (300.0 mg, 0.137 mmol) in ethanol (2 mL) was added hydroxylamine hydrochloride (247 mg, 3.55 mmol) and sodium bicarbonate (299 mg, 3.55 mmol). The resulting mixture was stirred at 90° C. overnight. The solid was filtered out. The filtrate was concentrated under vacuum to yield the title compound (28 B). Mass Spectrum (LCMS. ESI pos.): Calculated: For C₁₇H₂₁N₅O₂ (M⁺+H): 287.1, Measured: 287.1.

### C. N-(pivaloyloxy)-1-(pyrazin-2-yl)-1,4,5,6,7,8-hexahydro-5,8-epoxycyclohepta[c]pyrazole-3-carboximidamide (28 C).

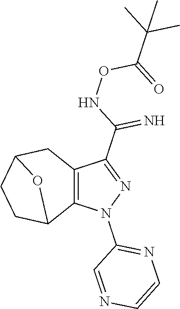 [SMILES: CC(C)(C)C(=O)ONC(=N)c1nn(-c2cnccn2)c2c1CC1CCC2O1]

To a solution of N-hydroxy-1-(pyrazin-2-yl)-1,4,5,6,7,8-hexahydro-5,8-epoxycyclohepta[c]pyrazole-3-carboximidamide (28 B) (150.0 mg, 0.524 mmol) in DCM (5 mL) was added pivaloyl chloride (75.8 mg, 0.629 mmol) and Et₃N (106.0 mg, 1.04 mmol). The resulting solution was then stirred at rt for 3 h and diluted with DCM (50 mL), washed with 2N HCl (16 mL×3). The combined DCM layers were dried over Na₂SO₄ and concentrated to yield N-(pivaloyloxy)-1-(pyrazin-2-yl)-1,4,5,6,7,8-hexahydro-5,8-epoxycyclohepta[c]pyrazole-3-carboximidamide (28 C) which was used in the next step without further purification.

### D. 3-(5-(tert-butyl)-1,2,4-oxadiazol-3-yl)-1-(pyrazin-2-yl)-1,4,5,6,7,8-hexahydro-5,8-epoxycyclohepta[c]pyrazole (Cpd. 32)

A solution N-(pivaloyloxy)-1-(pyrazin-2-yl)-1,4,5,6,7,8-hexahydro-5,8-epoxycyclohepta[c]pyrazole-3-carboximidamide (100.0 mg, 0.270 mmol) and toluene (5 mL) was heated at reflux for 5 h. The reaction mixture was cooled to rt and concentrated. The resulting residue was purified by reversed phase Prep-HPLC with Water (0.05% TEA)/ACN to yield the title compound 3-(5-(tert-butyl)-1,2,4-oxadiazol-3-yl)-1-(pyrazin-2yl)-1,4,5,6,7,8-hexahydro-5,8-epoxycyclohepta[c]pyrazole (Cpd. 32) after lyophilization. ¹H-NMR (300 MHz, CD₃OD) δ (ppm): 9.40 (s, 1H), 8.54 (d,1H), 8.49-8.50 (m, 1H), 5.93-5.95 (m, 1H), 4.86 (s, 1H), 3.18 (d, 1H), 2.66 (d, 1H), 2.27-2.33 (m, 3H), 1.70-1.73 (m, 1H), 1.54 (s, 9H). Mass Spectrum (LCMS. ESI pos.): Calculated: For C₁₈H₂₀N₆O₂ (M⁺+H): 353.2, Measured: 353.1.

### Example 29

### 3-(5-(tert-butyl)-1-methyl-1H-imidazol-2-yl)-1-(pyrazin-2-yl)-1,4,5,6,7,8-hexahydro-5,8-epoxycyclohepta[c]pyrazole (Cpd. 33)

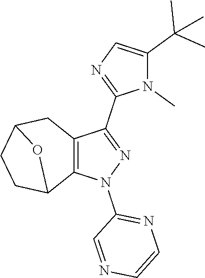 [SMILES: Cn1c(C(C)(C)C)cnc1-c1nn(-c2cnccn2)c2c1CC1CCC2O1]

### A. 1-(pyrazin-2-yl)-1,4,5,6,7,8-hexahydro-5,8-epoxycyclohepta[c]pyrazole-3-carboximidamide (29 A)

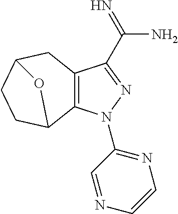 [SMILES: N=C(N)c1nn(-c2cnccn2)c2c1CC1CCC2O1]

To a solution of 1-(pyrazin-2-yl)-1,4,5,6,7,8-hexahydro-5,8-epoxycyclohepta[c]pyrazole-3-carbonitrile (28 A) (300.0 mg, 1.18 mmol) was added HCl (10 mL, 40 mmol, 4M in ethanol). The mixture was stirred at rt for 3 h and concentrated under vacuum. NH₃ (10 mL, 70 mmol, 7M in methanol) was then added and stirred for 10 min. The reaction was quenched with H₂O (100 mL). The resulting mixture was extracted with EtOAc (3×100 mL). The organic layers were combined, dried over Na₂SO₄, filtered and concentrated. The resulting residue was purified by silica gel chromatography (0-15% MeOH/DCM) to yield the title compound (26 A). Mass Spectrum (LCMS. ESI pos.): Calculated: For C₁₃H₁₄N₆O(M⁺+H): 271.1, Measured: 271.1.

### B. 3-(5-(tert-butyl)-1H-imidazol-2-yl)-1-(pyrazin-2-yl)-1,4,5,6,7,8-hexahydro-5,8-epoxycyclohepta[c]pyrazole (29 B)

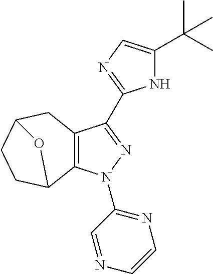 [SMILES: CC(C)(C)c1cnc(-c2nn(-c3cnccn3)c3c2CC2CCC3O2)[nH]1]

To a solution of 1-(pyrazin-2-yl)-1,4,5,6,7,8-hexahydro-5,8-epoxycyclohepta[c]pyrazole-3-carboximidamide (29 A) (190.0 mg, 0.703 mmol) in THF: water (4:1, 2.5 mL) was added 4-bromo-2,2-dimethylpentan-3-one (126 mg, 0.703 mmol) and K₂CO₃ (97.2 mg, 0.703 mmol). The mixture was stirred at 70° C. for 3 h and treated with H₂O (100 mL). The resulting mixture was extracted with EtOAc (100 mL×3). The organic layers were combined, dried over Na₂SO₄, filtered and concentrated. The resulting residue was purified by silica gel chromatography (5-40% Ethyl acetate/Petroleum ether) to yield the title compound (29 B). Mass Spectrum (LCMS. ESI pos.): Calculated: For C₁₉H₂₂N₆O (M⁺+H): 351.2, Measured: 351.1.

### C. 3-(5-(tert-butyl)-1-methyl-1H-imidazol-2-yl)-1-(pyrazin-2-yl)-1,4,5,6,7,8-hexahydro-5,8-epoxycyclohepta[c]pyrazole (Cpd. 33)

To a solution of 3-(5-(tert-butyl)-1H-imidazol-2-yl)-1-(pyrazin-2-yl)-1,4,5,6,7,8-hexahydro-5,8-epoxycyclohepta[c]pyrazole (29 B) (50.0 mg, 0.143 mmol) in THF (3 mL) was added NaH (10.3 mg, 0.428 mmol) at 0° C. The resulting mixture was stirred at 0° C. for 0.5 h, followed by the addition of iodomethane (60.7 mg, 0.428 mmol). The reaction was quenched with satd, NH₄Cl (100 mL). The resulting mixture was extracted with EtOAc (100 mL×3). The organic layers were combined, dried over Na₂SO₄, filtered and concentrated. The resulting product was then purified by reversed phase Prep-HPLC using H₂O (0.05% NH₄HCO₃) and CH₃CN to yield the 3-(5-(tert-butyl)-1-methyl -1H-imidazol-2-yl)-1-(pyrazin-2-yl)-1,4,5,6,7,8-hexahydro-5,8-epoxycyclohepta[c]pyrazole (Cpd. 33). ¹H-NMR (300 MHz, DMSO-d₆) δ (ppm): 9.28 (s, 1H), 8.55-8.58 (m, 1H), 8.53-8.54 (m, 1H), 6.97 (s, 1H), 5.85 (s, 1H), 4.76-4.80 (m, 1H), 3.98 (s, 3H), 3.10-3.17 (m, 1H), 2.67-2.71 (m, 1H), 2.16 (s, 3H), 1.59-1.67 (m, 1H), 1.25 (s, 9H). Mass Spectrum (LCMS. ESI pos.): Calculated: For C₂₀H₂₄N₆O (M⁺+H): 365.2, Measured: 365.2.

### Example 30

### 3-(5-(tert-butyl)thiazol-2-yl)-1-(pyrazin-2-yl)-1,4,5,6,7,8-hexahydro-5,8-epoxycyclohepta[c]pyrazole (Cpd. 34)

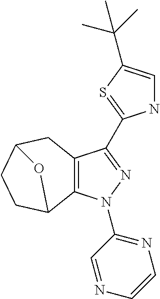

### A. N-(3,3-dimethyl-2-oxobuty)-1-(pyrazin-2-yl)-1,4,5,6,7,8-hexahydro-5,8-epoxycyclohepta[c]pyrazole-3-carboxamide (30 A)

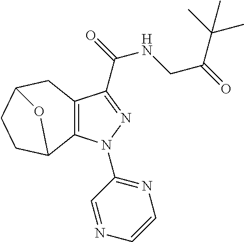 [SMILES: CC(C)(C)C(=O)CNC(=O)c1nn(-c2cnccn2)c2c1CC1CCC2O1]

To a solution of 1-(pyrazin-2-yl)-1,4,5,6,7,8-hexahydro-5,8-epoxycyclohepta[c]pyrazole-3-carboxylic acid (2 B) (300.0 mg, 1 mmol) in DCM (5 mL) was added HATU (838 mg, 2.20 mmol) and DEA (285 mg, 2.20 mmol), followed by 1-amino-3,3-dimethylbutan-2-one (254 mg, 2.20 mmol). The mixture was stirred at rt for 3 h. The reaction was quenched with H₂O (100 mL). The resulting mixture was extracted with EtOAc (3×100 mL). The organic layers were combined, dried over Na₂SO₄, filtered and concentrated. The resulting residue was purified by silica gel chromatography (5-10% EtOAc/heptane) to yield the title compound (30 A). Mass Spectrum (LCMS. ESI pos.): Calculated: For C₁₉H₂₃N₅O₃ (M⁺+H): 370.2, Measured: 370.2.

### B. 3-(5-(tert-butyl)thiazol-2-yl)-1-(pyrazin-2-yl)-1,4,5,6,7,8-hexahydro-5,8-epoxycyclohepta[c]pyrazole (Cpd. 34)

To a solution of N-(3,3-dimethyl-2-oxobutyl)-1-(pyrazin-2-yl)-1,4,5,6,7,8-hexahydro-5,8 epoxycyclohepta[c]pyrazole-3-carboxamide (30 A) (20.0 mg, 0.054 mmol) in toluene (1 mL) was added Lawesson's Reagent (29 mg, 0.07 mmol). The mixture was stirred at 120° C. overnight. The reaction was allowed to cool to RT and quenched with H₂O (50 mL). The resulting mixture was extracted with EtOAc (3×100 mL). The organic layers were combined, dried over Na₂SO₄, filtered and concentrated. The resulting product was then purified by reversed phase Prep-HPLC using H₂O (0.05% NH₄HCO₃) and CH₃CN to yield 3-(5-(tert-butyl)thiazol-2-yl)-1-(pyrazin-2-yl) -1,4,5,6,7,8-hexahydro-5,8-epoxycyclohepta[c]pyrazole (Cpd. 34). ¹H-NMR (400 MHz, CD₃OD) δ (ppm): 9.31 (s, 1H), 8.49-8.52 (m, 2H), 7.63 (s, 1H), 5.95-5.96 (m, 1H), 4.87 (s, 1H), 3.25-3.29 (m, 1H), 2.73-2.77 (m, 1H), 2.28-2.33 (m, 3H), 1.72-1.76 (m, 1H), 1.47 (s, 9H). Mass Spectrum (LCMS. ESI pos.): Calculated: For C₁₉H₂₁N₅OS (M⁺+H): 368.2, Measured: 368.1.

### Example 31

### 3-(4-(tert-butypthiazol-2-yl)-1-(pyrazin-2-yl)-1,4,5,6,7,8-hexahydro-5,8-epoxycyclohepta[c]pyrazole (Cpd. 35)

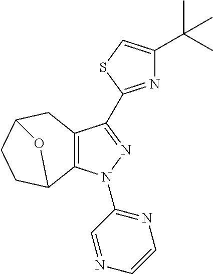 [SMILES: CC(C)(C)c1csc(-c2nn(-c3cnccn3)c3c2CC2CCC3O2)n1]

### A. 1-(pyrazin-2-yl)-1,4,5,6,7,8-hexahydro-5,8-epoxycyclohepta[c]pyrazole-3-carbothioamide (31 A)

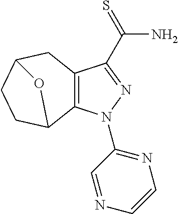 [SMILES: NC(=S)c1nn(-c2cnccn2)c2c1CC1CCC2O1]

To a solution of 1-(pyrazin-2-yl)-1,4,5,6,7,8-hexahydro-5,8-epoxycyclohepta[c]pyrazole-3-carboxamide (37 A) (300.0 mg, 1.11 mmol) in 1,4-dioxane (10 mL) was added Lawesson's Reagent (581 mg, 1.44 mmol). The mixture was stirred at 100° C. for 3 h. The reaction mixture was allowed to cool to RT and treated with H₂O (50 mL). The resulting mixture was extracted with EtOAc (3×100 mL). The organic layers were combined, dried over Na₂SO₄, filtered and concentrated. The resulting residue was purified by silica gel chromatography (1-5% DCM/MeOH) to yield the title compound (31 A). Mass Spectrum (LCMS. ESI pos.): Calculated: For C₁₃H₁₃N₅OS (M⁺+H): 388.1, Measured: 288.1.

### B. 3-(4-(tert-butyl)thiazol-2yl)-1-(pyrazin-2-yl)-1,4,5,6,7,8-hexahydro-5,8-epoxycyclohepta[c]pyrazole (Cpd. 35)

To a solution of 1-(pyrazin-2-yl)-1,4,5,6,7,8-hexahydro-5,8-epoxycyclohepta[c]pyrazole-3-carbothioamide (31 A) (200.0 mg, 0.696 mmol) in ethanol (5 mL) was added 4-bromo-2,2-dimethylpentan-3-one (201 mg, 1.04 mmol). The mixture was stirred at 70° C. for 3 h. The reaction was quenched with H₂O (100 mL) after cooling to RT. The resulting mixture was extracted with EtOAc (3×100 mL). The organic layers were combined, dried over Na₂SO₄, filtered and concentrated. The resulting residue was then purified by reversed phase Prep-HPLC using H₂O (0.05% NH₄HCO₃) and CH₃CN to yield the 3-(4-(tert-butyl)thiazol-2-yl)-1-(pyrazin-2-yl) -1,4,5,6,7,8-hexahydro-5,8-epoxycyclohepta[c]pyrazole (Cpd. 35). ¹H-NMR (400 MHz, DMSO-d₆) δ (ppm): 9.22 (s, 1H), 8.57-8.62 (d, 2H), 7.35 (s, 1H), 5.84 (s. 1H), 4.82 (s, 1H), 3.17-3.22 (m, 1H), 2.68-2.74 (m, 1H), 2.23-2.35 (m, 3H), 1.66-1.67 (m, 1H), 1.35 (s, 9H). Mass Spectrum (LCMS. ESI pos.): Calculated: For C₁₉H₂₁N₅OS (M⁺+H): 368.1, Measured: 268.1.

### Example 32

### 3-(3-(tert-pentyl)-1,2,4-oxadiazol-5-yl)-1-(pyrazin-2-yl)-1,4,5,6,7,8-hexahydro-5,8-epoxycyclohepta[c]pyrazole (Cpd, 36)

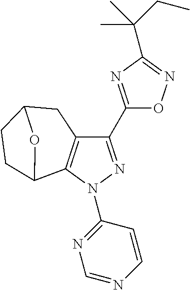 [SMILES: CCC(C)(C)c1noc(-c2nn(-c3ccncn3)c3c2CC2CCC3O2)n1]

### A. N-hydroxy-2,2-dimethylbutanimidamide (32 A)

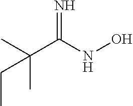

To a solution of 2,2-dimethylbutanenitrile (1.0 g, 0.01 mol) in ethanol (15 mL) was added hydroxylamine hydrochloride (2.1 g, 0.03 mol) and NaHCO₃ (2.6 g, 0.03 mol) at r.t. The resulting mixture was stirred at 90° C. overnight. The reaction mixture was allowed to cool to RT, filtered and concentrated to yield N-hydroxy-2,2-dimethylbutanimidamide. Mass Spectrum (LCMS. ESI pos.): Calculated: For C₆H₁₄N₂O (M⁺+H): 131.1, Measured: 131.2.

### B. 3-(3-(tert-pentyl)-1,2,4-oxadiazol-5-yl)-1-(pyrazin-2-yl)-1,4,5,6,7,8-hexahydro-5,8-epoxycyclohepta[c]pyrazole (Cpd. 36)

To a solution of 1-(pyrazin-2-yl)-1,4,5,6,7,8-hexahydro-5,8-epoxycyclohepta[c]pyrazole-3-carboxylic acid (2 B) (150.0 mg, 0.550 mmol) and HATU (419 mg, 1.10 mmol) in DMF (10 mL) was added DIEA (142 mg, 1.10 mmol). The resulting mixture was stirred at RT for 5 min. and treated with N-hydroxy-2,2-dimethylbutanimidamide (32 A) (143 mg, 1.10 mmol). The resulting mixture was stirred at 40° C. overnight and at 120° C. for 2 h. After cooling down to r.t., the reaction was quenched with water (10 ml). The resulting mixture was extracted with ethyl acetate (3×50 ml). The organic layers were combined, dried over anhydrous sodium sulfate, filtered and concentrated. The resulting residue was purified reversed phase Prep-HPLC using H₂O (0.05% NH₄HCO₃) and CH₃CN to yield the 3-(3-(tert-pentyl)-1,2,4-oxadiazol-5-yl) -1-(pyrazin-2-yl)-1,4,5,6,7,8-hexahydro-5,8-epoxycyclohepta[c]pyrazole (Cpd. 36). ¹H-NMR (300 MHz, DMSO-d₆) δ (ppm): 9.28 (d, 1H), 8.71-8.72 (m, 1H), 8.63 (dd, 1H), 5.83-5.85 (m, 1H), 4.77-4.86 (m, 1H), 3.14-3.22 (m, 1H), 2.66-2.72 (m, 1H), 2.17-2.29 (m, 3H), 1.70-1.78 (m, 3H), 1.35-1.38 (m, 6H), 0.78-0.81 (m, 3H). Mass Spectrum (LCMS. ESI pos.): Calculated: For C₁₉H₂₂N₆O₂ (M⁺+H): 367.2, Measured: 367.2.

### Example 33

### 3-(3-(tert-butyl)8-hexahydro-5,8-epoxycyclohepta[c]pyrazole (Cpd. 37)

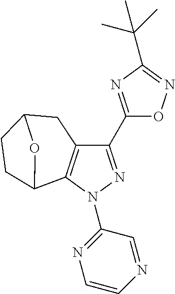 [SMILES: CC(C)(C)c1noc(-c2nn(-c3cnccn3)c3c2CC2CCC3O2)n1]

To a stirred solution of 1-(pyrazin-2-yl)-1,4,5,6,7,8-hexahydro-5,8-epoxycyclohepta[c]pyrazole-3-carboxylic acid (2 B) (150.0 mg, 0.551 mmol) and HATU (419 mg, 1.10 mmol) in DMF (10 mL) was added ©IEA (142 mg, 1.10 mmol). The resulting mixture was stirred for 5 min and treated with N′-hydroxypivalimidamide (128 mg, 1.10 mmol). The resulting mixture was stirred at 40° C. overnight followed by stirring 120° C. for 2 h. After cooling down to r.t., the reaction was quenched with water (10 mL). The resulting mixture was extracted with ethyl acetate (3×50 ml). The organic layers were combined, dried over anhydrous sodium sulfate, filtered and concentrated. The resulting residue was purified by reversed phase Prep-HPLC using H₂O (0.05% NH₄HCO₃) and CH₃CN to yield the 3-(3-(tert-butyl)-1,2,4-oxadiazol-5-yl)-1-(pyrazin-2-yl)-1,4,5,6,7,8-hexahydro-5,8-epoxycyclohepta[c]pyrazole (Cpd. 37). ¹H-NMR (300 MHz, DMSO-d₆) δ (ppm): 9.28 (d, 1H), 8.70-8.72 (m, 1H), 8.65 (dd, 1H), 5.83-5.85 (m, 1H), 4.77-4.86 (m, 1H), 3.16 (dd, 1H), 2.66-2.72 (m, 1H), 2.17-2.26 (m, 3H), 1.61-1.79 (m, 1H), 1.33-1.43 (m, 9H). Mass Spectrum (LCMS. ESI pos.): Calculated: For C₁₈H₂₀N₆O₂ (M⁺+H): 353.1, Measured: 353.2.

### Example 34

### 3-(5-(tert-butyl)-1,3,4-thiadiazol-2-yl)-1-(pyrazin-2-yl)-1,4,5,6,7,8-hexahydro-5,8-epoxycyclohepta[c]pyrazole (Cpd. 38)

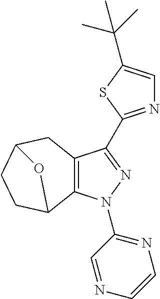 [SMILES: CC(C)(C)c1cnc(-c2nn(-c3cnccn3)c3c2CC2CCC3O2)s1]

### A. N′-pivaloyl-1-(pyrazin-2-yl)-1,4,5,6,7,8-hexahydro-5,8-epoxycyclohepta[c]pyrazole-3-carbohydrazide (34 A)

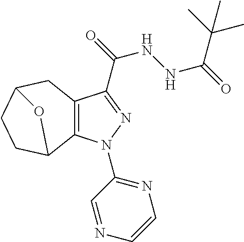 [SMILES: CC(C)(C)C(=O)NNC(=O)c1nn(-c2cnccn2)c2c1CC1CCC2O1]

To a solution of 1-(pyrazin-2-yl)-1,4,5,6,7,8-hexahydro-5,8-epoxycyclohepta[c]pyrazole-3-carboxylic acid (2 B) (0.5 g, 2 mmol) in DCM (10 mL) was added HATU (1.4 g, 3.6 mmol) and DIEA (0.5 g, 3.6 mmol), followed by the addition of pivalohydrazide (0.4 g, 3.6 mmol). The mixture was stirred at rt for 3 h. The reaction was quenched with H₂O (100 mL). The resulting mixture was extracted with EtOAc (3×100 mL). The organic layers were combined, dried over Na₂SO₄, filtered and concentrated. The resulting residue was purified by silica gel chromatography (5-10% EtOAc/heptane) to yield the title compound. Mass Spectrum (LCMS. ESI pos.): Calculated: For C₁₈H₂₂N₆O₃ (M⁺+H); 371.2, Measured: 371.1.

### B. 3-(5-(tert-butyl)-1,3,4-thiadiazol-2-yl)-1-(pyrazin-2-yl)-1,4,5,6,7,8-hexahydro-5,8-epoxycyclohepta[c]pyrazole (Cpd. 38)

To a solution of N′-pivaloyl-1-(pyrazin-2-yl)-1,4,5,6,7,8-hexahydro-5,8-epoxycyclohepta[c]pyrazole-3-carbohydrazide (34 A) (200.0 mg, 0.540 mmol) in toluene (3 mL) was added Lawesson's Reagent (284 mg, 0.702 mmol). The mixture was stirred at 120° C. overnight. The reaction was quenched with H₂O (50 mL) after cooling to rt. The resulting mixture was extracted with EtOAc (3×100 mL). The organic layers were combined, dried over Na₂SO₄, filtered and concentrated. The resulting residue was then purified by reversed phase Prep-HPLC using H₂O (0.05% NH₄HCO₃) and CH₃CN to yield the 3-(5-(tert-butyl)-1,3,4-thiadiazol-2-yl)-1-(pyrazin-2-yl)-1,4,5,6,7,8-hexahydro -5,8-epoxycyclohepta[c]pyrazole (Cpd. 38). ¹H-NMR (400 MHz, DMSO-d₆) δ (ppm): 9.21 (s, 1H), 8.66 (s, 1H), 8.59 (s, 1H), 5.83-5.84 (m, 1H), 4.82 (s, 1H), 3.16-3.22 (m, 1H), 2.69-2.73 (m, 1H), 2.18-2.25 (m, 3H), 1.64-1.68 (m, 1H), 1.48 (s, 9H). Mass Spectrum (LCMS. ESI pos.): Calculated: For C₁₈H₂₀N₆OS (M⁺+H): 369.1, Measured: 369.1.

### Example 35

### 3-(5-(tert-butyl)-1,3,4-oxadiazol-2-yl)-1-(pyrazin-2-yl)-1,4,5,6,7,8-hexahydro-5,8-epoxycyclohepta[c]pyrazole (Cpd. 39)

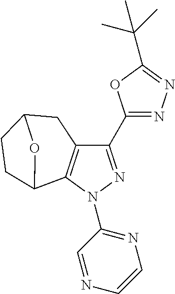 [SMILES: CC(C)(C)c1nnc(-c2nn(-c3cnccn3)c3c2CC2CCC3O2)o1]

To a solution of N′-pivaloyl-1-(pyrazin-2-yl)-1,4,5,6,7,8-hexahydro-5,8-epoxycyclohepta[c]pyrazole-3-carbohydrazide (34 A) (250.0 mg, 0.675 mmol) in toluene (3 mL) was added Burgess reagent (482 mg, 2.02 mmol). The mixture was stirred at 120° C. for 3 h. After cooling to rt the reaction was quenched with H₂O (50 mL). The resulting mixture was extracted with EtOAc (3×100 mL). The organic layers were combined, dried over Na₂SO₄, filtered and concentrated. The resulting residue was then purified by reversed phase Prep-HPLC using H₂O (0.05% NH₄HCO₃) and CH₃CN to yield 3-(5-(tert-butyl)-1,3,4-oxadiazol-2-yl)-1-(pyrazin-2-yl)-1,4,5,6,7,8-hexahydro-5,8-epoxycyclohepta[c]pyrazole (Cpd. 39). ¹H-NMR (400 MHz, CD₃OD) δ (ppm): 9.37 (s, 1H), 8.59 (s, 1H), 8.53 (s, 1H), 5.95-5.96 (m, 1H), 4.87 (s, 1H), 3.25-3.29 (m, 1H), 2.71-2.76 (m, 1H), 2.29-2.35 (m, 3H), 1.72-1.76 (m, 1H), 1.52 (s, 9H). Mass Spectrum (LCMS. ESI pos.): Calculated: For C₁₈H₂₀N₆O₂ (M⁺+H): 353.2, Measured: 353.1.

### Example 36

### 3-(5-(tert-butyl)oxazol-2-yl)-1-(pyrazin-2-yl)-1,4,5,6,7,8-hexahydro-5,8-epoxycyclohepta[c]pyrazole (Cpd. 40)

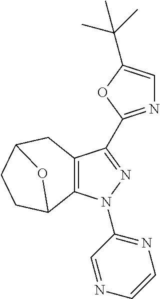 [SMILES: CC(C)(C)c1cnc(-c2nn(-c3cnccn3)c3c2CC2CCC3O2)o1]

### A. N-(3,3-dimethyl-2-oxobutyl)-1-(pyrazin-2-yl)-1,4,5,6,7,8-hexahydro-5,8-epoxycyclohepta[c]pyrazole-3-carboxamide (36 A)

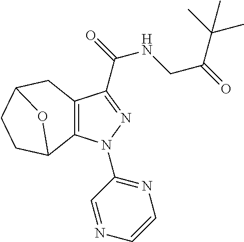 [SMILES: CC(C)(C)C(=O)CNC(=O)c1nn(-c2cnccn2)c2c1CC1CCC2O1]

To a solution of 1-(pyrazin-2-yl)-1,4,5,6,7,8-hexahydro-5,8-epoxycyclohepta[c]pyrazole-3-carboxylic acid (2 B) (200.0 mg, 0.735 mmol) in DCM (5 mL) was added HATU (419 mg, 1.10 mmol), ethyldiisopropylamine (380 mg, 2.9 mmol) and 1-amino-3,3-dimethylbutan-2-one (223 mg, 1.47 mmol). The resulting mixture was stirred at rt overnight. The reaction was quenched with H₂O (20 mL), the resulting mixture was extracted with DCM (3×15 mL). The organic layers were combined, dried over Na₂SO₄, filtered and concentrated. The resulting residue was purified by silica gel chromatography (30-40% EtOAc/petroleum ether) to yield the title compound (36 A). Mass Spectrum (LCMS. ESI pos.): Calculated: For C₁₉H₂₃N₅O₃ (M⁺+H): 370.1, Measured: 370.2.

### B. 3-(5-(tert-butypoxazol-2-yl)-1-(pyrazin-2-yl)-1,4,5,6,7,8-hexahydro-5,8 epoxycyclohepta[c]pyrazole (Cpd. 40)

To a solution of N-(3,3-dimethyl-2-oxobutyl)-1-(pyrazin-2-yl)-1,4,5,6,7,8-hexahydro-5,8-epoxycyclohepta[c]pyrazole-3-carboxamide (36 A) (200.0 mg, 0.541 mmol) in toluene (8 mL) was added Burgess reagent (258 mg, 1.08 mmol). The resulting mixture was stirred at 110° C. overnight. After cooling to r.t., the solvent was removed under vacuum. The resulting residue was purified by reverse-phase flash chromatography using CH₃CN:H₂O (0.05% NH₄HCO₃) to yield 3-(5-(tert-butypoxazol-2-yl)-1-(pyrazin-2-yl)-1,4,5,6,7,8-hexahydro-5,8-epoxycyclohepta[c]pyrazole (Cpd. 40). ¹H-NMR (400 MHz, DMSO-d₆) δ (ppm): 9.24 (d, 1 H), 8.64 (d, 1 H), 8.58 (dd, 1 H), 7.05 (s, 1H), 5.80 5.83 (m, 1H), 4.78-4.82 (m, 1 H), 3.11-3.17 (m, 1 H), 2.62-2.65 (m, 1H), 2.12-2.21 (m, 3H), 1.62-1.70 (m, 1H), 1.34 (s, 9H). Mass Spectrum (LCMS. ESI pos.): Calculated: For C₁₉H₂₁N₅O₂ (M⁺+H): 352.1, Measured: 352.2.

### Example 37

### 1-(pyrazin-2-yl)-1,4,5,6,7,8-hexahydro-5,8-epoxycyclohepta[c]pyrazole-3-carboxamide

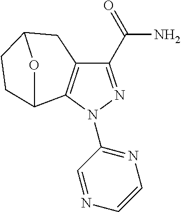 [SMILES: NC(=O)c1nn(-c2cnccn2)c2c1CC1CCC2O1]

1-(pyrazin-2-yl)-1,4,5,6,7,8-hexahydro-5,8-epoxycyclohepta[c]pyrazole-3-carboxamide (37 A) was prepared by reacting 1-(pyrazin-2-yl)-1,4,5,6,7,8-hexahydro -5,8-epoxycyclohepta[c]pyrazole-3-carboxylic acid (2 B) with NH₄Cl, according to the procedure described in Example 1, Step D. Mass Spectrum (LCMS. ESI pos.): Calculated. For C₁₃H₁₃N₅O₂ (M⁺+H): 272.1, Measured: 272.0.

### Biological Example 1

### Method for Assaying Agonism of CB1 or CB2 by Detection of cAMP Using Homogeneous Time-Resolved Fluorescence (HTRF®)

CHOP-K1 cells stably expressing the CB1 receptor (PerkinElmer, ES-110-C) or the CB2 receptor (PerkinElmer, ES-111-CF) were seeded into 384 well white TC-treated plates (Greiner 781080) at 7000 cells per well in Ham's media (Gibco #31765-035) supplemented with 10% FBS (Gibco #16000-044) and 500 μg/mL Geneticin (Gibco #10131-027) and cultured overnight. The next day, plates were washed one time in assay buffer (HBSS with Ca²⁺ and Mg²⁺ (Corning #21-023-CV)+20 mM HEPES (Corning #25-060-Cl) and 0.01% BSA (Sigma A2153)). In a separate compound plate (Greiner 781201-906), 50 nL of test compound or DMSO was transferred using the Echo 550 (Labcyte), then 40 μL/well of assay buffer with 0.5 mM IBMX, 2 μM Forskolin (Sigma F3917), and 0.01% BSA was added. For the CB1 assay, assay buffer was dumped from the cell plate and fresh assay buffer containing 0.5 mM IBMX (Sigma I5679) was added at 30 μL/well, and the plate was incubated for 1 hr at 37° C., 5% CO₂. Following the IBMX pre-treatment step, buffer was dumped from the cell plate, and 20 μL/well of the assay buffer with test compound was transferred into the cell plate with a subsequent incubation of 20 min at room temperature. Reagents for cAMP detection (cisbio 62AM4PEJ) were prepared and subsequently transferred to the cell plate at 20 μL/well. Following a 1 hr incubation at room temperature, the plate was read on the Pherastar FX (BMG, excitation 320 nm, detection at 620 nm (donor) and 665 nm (acceptor)). The ratio of signal at 665 nm/620 nm was determined for each well in the plate. For each assay day, a cAMP standard curve was run in parallel to assess the range of response and determine the concentration of cAMP in each well. Control wells (n=16 of each) with no activation of CB1 receptor (DMSO) and maximal activation (5 nM CP55940, Sigma) were used to normalize the data and to determine signal-to-background ratio and Z′. For the CB2 assay, conditions were as described above for the CB1 assay, except that the IBMX preincubation step was not used and that the 20 min compound incubation step occurred in the incubator at 37° C., 5% CO₂.

EC₅₀ values were calculated from dose response curves by nonlinear regression analysis using PRISM software.

Representative compounds of of the present invention were tested according to the procedures described in Biological Example 1 above, with results as listed in Table 3, below. If a compound was tested more than once (due to multiple samples or batches), then the value listed in Table 3 below represents the average of the measured values. For a measured EC₅₀>5 μM and/or % Eₘₐₓ<50, the compound is denoted as “LA” in the table below.

TABLE 3
Measured CB1 and CB2 Activity

| | | hCB₂ CHO | hCB₁ CHO |
|---|---|---|---|
| Compound # | Structure | EC₅₀ nM (Eₘₐₓ %) | EC₅₀ nM (Eₘₐₓ %) |
| 1 | 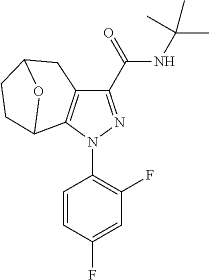 [SMILES: CC(C)(C)NC(=O)c1nn(-c2ccc(F)cc2F)c2c1CC1CCC2O1] | 1.2 (95.6) | 107.5 (99) |
| 2 | 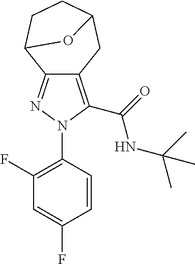 [SMILES: CC(C)(C)NC(=O)c1c2c(nn1-c1ccc(F)cc1F)C1CCC(C2)O1] | 2980 (72.8) | LA |
| 3 | 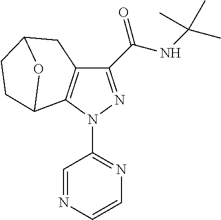 [SMILES: CC(C)(C)NC(=O)c1nn(-c2cnccn2)c2c1CC1CCC2O1] | 3.8 (99.7) | 2080 (94.8) |
| 4 | 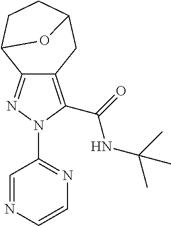 [SMILES: CC(C)(C)NC(=O)c1c2c(nn1-c1cnccn1)C1CCC(C2)O1] | 4070 (75.8) | LA |
| 5 | 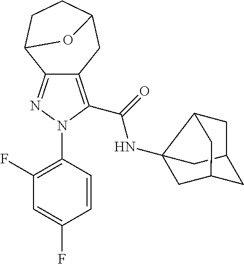 [SMILES: O=C(NC12CC3CC(CC1C3)C2)c1c2c(nn1-c1ccc(F)cc1F)C1CCC(C2)O1] | LA | LA |
| 6 | 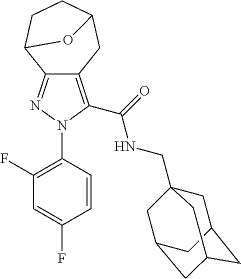 [SMILES: O=C(NCC12CC3CC(CC(C3)C1)C2)c1c2c(nn1-c1ccc(F)cc1F)C1CCC(C2)O1] | LA | LA |
| 7 | 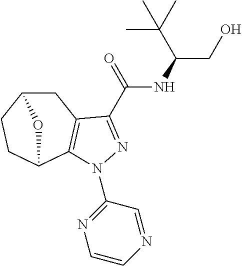 [SMILES: CC(C)(C)[C@@H](CO)NC(=O)c1nn(-c2cnccn2)c2c1C[C@@H]1CC[C@H]2O1] | 0.56 (100.5) | LA |
| 8 | 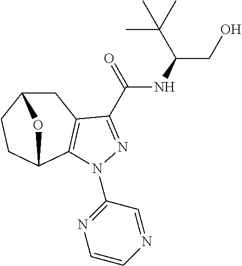 [SMILES: CC(C)(C)[C@@H](CO)NC(=O)c1nn(-c2cnccn2)c2c1C[C@H]1CC[C@@H]2O1] | 0.09 (100.1) | 422 (111.1) |
| 9 | 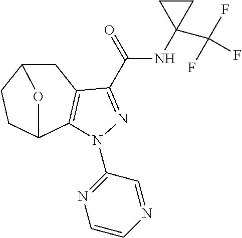 [SMILES: O=C(NC1(C(F)(F)F)CC1)c1nn(-c2cnccn2)c2c1CC1CCC2O1] | 0.24 (100.4) | LA |
| 10 | 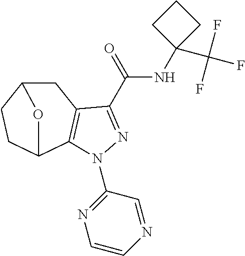 [SMILES: O=C(NC1(C(F)(F)F)CCC1)c1nn(-c2cnccn2)c2c1CC1CCC2O1] | 0.04 (100.3) | 571 (90.3) |
| 11 | 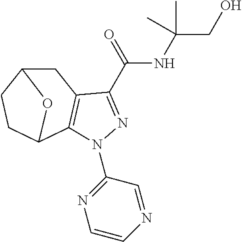 [SMILES: CC(C)(CO)NC(=O)c1nn(-c2cnccn2)c2c1CC1CCC2O1] | 7.1 (99.2) | LA |
| 12 | 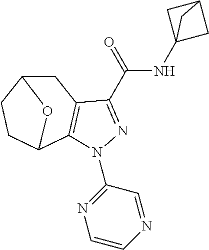 [SMILES: O=C(NC12CC(C1)C2)c1nn(-c2cnccn2)c2c1CC1CCC2O1] | 0.24 (99.5) | 1505 (90.8) |
| 13 | 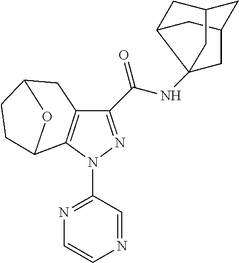 [SMILES: O=C(NC12CC3CC(CC1C3)C2)c1nn(-c2cnccn2)c2c1CC1CCC2O1] | 0.02 (100.1) | 16.6 (111.4) |
| 14 | 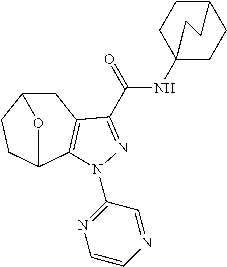 [SMILES: O=C(NC12CCC(CC1)CC2)c1nn(-c2cnccn2)c2c1CC1CCC2O1] | 0.06 (100.4) | 61.8 (102.8) |
| 15 | 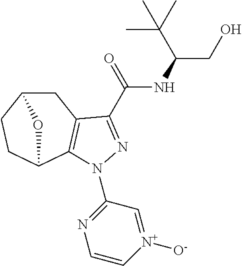 [SMILES: CC(C)(C)[C@@H](CO)NC(=O)c1nn(-c2c[n+]([O-])ccn2)c2c1C[C@@H]1CC[C@H]2O1] | 0.34 (100.7) | LA |
| 16 | 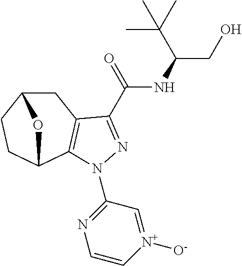 [SMILES: CC(C)(C)[C@@H](CO)NC(=O)c1nn(-c2c[n+]([O-])ccn2)c2c1C[C@H]1CC[C@@H]2O1] | 0.09 (100.7) | 352 (91.3) |
| 17 | 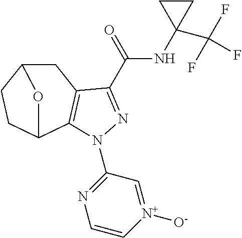 [SMILES: O=C(NC1(C(F)(F)F)CC1)c1nn(-c2c[n+]([O-])ccn2)c2c1CC1CCC2O1] | 0.12 (100.2) | LA |
| 18 | 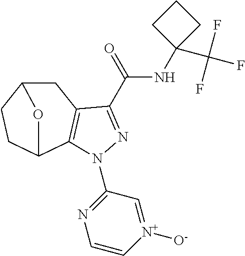 [SMILES: O=C(NC1(C(F)(F)F)CCC1)c1nn(-c2c[n+]([O-])ccn2)c2c1CC1CCC2O1] | 0.04 (99.9) | 198 (90.7) |
| 19 | 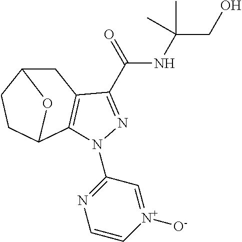 [SMILES: CC(C)(CO)NC(=O)c1nn(-c2c[n+]([O-])ccn2)c2c1CC1CCC2O1] | 1.0 (100.3) | LA |
| 20 | 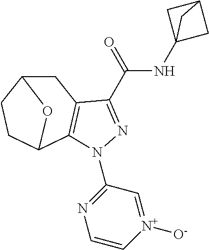 [SMILES: O=C(NC12CC(C1)C2)c1nn(-c2c[n+]([O-])ccn2)c2c1CC1CCC2O1] | 0.05 (100.2) | 1781 (91) |
| 21 | 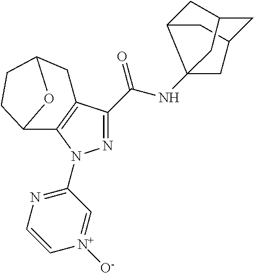 [SMILES: O=C(NC12CC3CC(CC1C3)C2)c1nn(-c2c[n+]([O-])ccn2)c2c1CC1CCC2O1] | 0.02 (99.9) | 28.2 (94.9) |
| 22 | 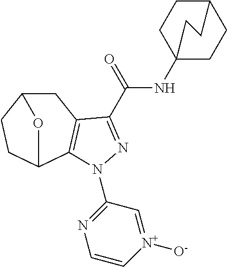 [SMILES: O=C(NC12CCC(CC1)CC2)c1nn(-c2c[n+]([O-])ccn2)c2c1CC1CCC2O1] | 0.02 (99.7) | 48.5 (96.5) |
| 23 | 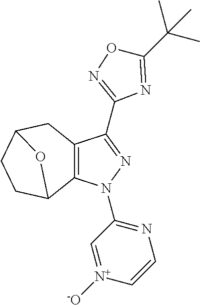 | 0.10 (100.3) | LA |
| 24 | 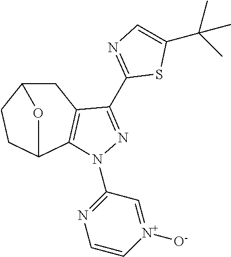 [SMILES: CC(C)(C)c1cnc(-c2nn(-c3c[n+]([O-])ccn3)c3c2CC2CCC3O2)s1] | 0.06 (97.8) | 65.7 (95.5) |
| 25 | 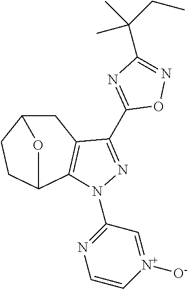 [SMILES: CCC(C)(C)c1noc(-c2nn(-c3c[n+]([O-])ccn3)c3c2CC2CCC3O2)n1] | 0.02 (99.5) | 113.6 (98.9) |
| 26 | 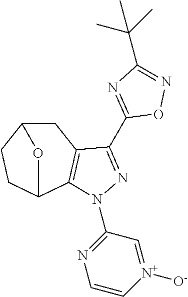 [SMILES: CC(C)(C)c1noc(-c2nn(-c3c[n+]([O-])ccn3)c3c2CC2CCC3O2)n1] | 0.13 (100.1) | 4917 (73.3) |
| 27 | 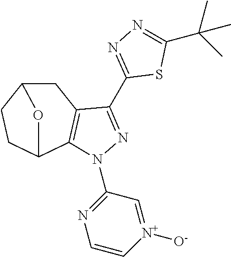 [SMILES: CC(C)(C)c1nnc(-c2nn(-c3c[n+]([O-])ccn3)c3c2CC2CCC3O2)s1] | 0.06 (99) | 130.3 (94.9) |
| 28 | 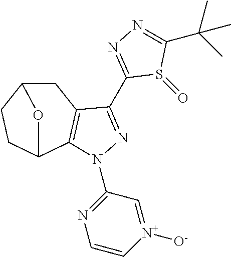 [SMILES: CC(C)(C)C1=NN=C(c2nn(-c3c[n+]([O-])ccn3)c3c2CC2CCC3O2)S1=O] | 0.74 (99) | LA |
| 29 | 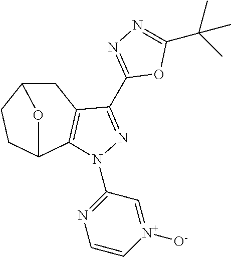 [SMILES: CC(C)(C)c1nnc(-c2nn(-c3c[n+]([O-])ccn3)c3c2CC2CCC3O2)o1] | 0.20 (100.7) | 2372 (79.3) |
| 30 | 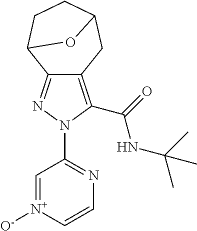 [SMILES: CC(C)(C)NC(=O)c1c2c(nn1-c1c[n+]([O-])ccn1)C1CCC(C2)O1] | 7974 (66.3) | LA |
| 31 | 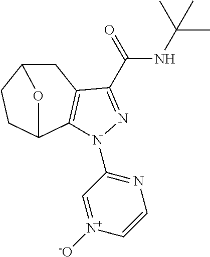 | 0.33 (99.7) | 1429 (83.2) |
| 32 | 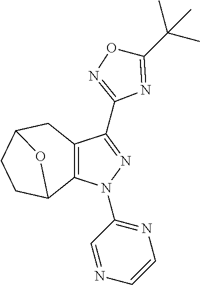 [SMILES: CC(C)(C)c1nc(-c2nn(-c3cnccn3)c3c2CC2CCC3O2)no1] | 0.33 (100.4) | LA |
| 33 | 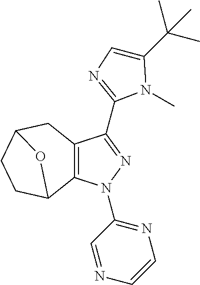 [SMILES: Cn1c(C(C)(C)C)cnc1-c1nn(-c2cnccn2)c2c1CC1CCC2O1] | LA | LA |
| 34 | 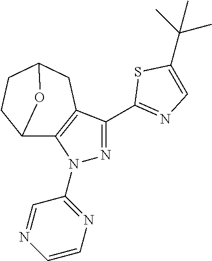 [SMILES: CC(C)(C)c1cnc(-c2nn(-c3cnccn3)c3c2CC2CCC3O2)s1] | 0.24 (99.7) | LA |
| 35 | 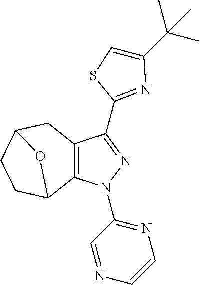 [SMILES: CC(C)(C)c1csc(-c2nn(-c3cnccn3)c3c2CC2CCC3O2)n1] | LA | LA |
| 36 | 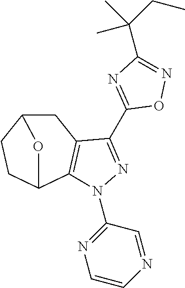 [SMILES: CCC(C)(C)c1noc(-c2nn(-c3cnccn3)c3c2CC2CCC3O2)n1] | 0.04 (99.8) | LA |
| 37 | 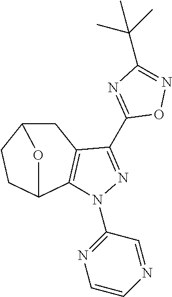 [SMILES: CC(C)(C)c1noc(-c2nn(-c3cnccn3)c3c2CC2CCC3O2)n1] | 0.22 (99.2) | 177 (34.8) |
| 38 | 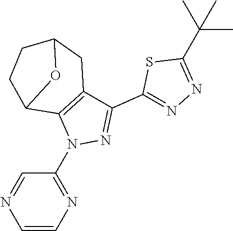 [SMILES: CC(C)(C)c1nnc(-c2nn(-c3cnccn3)c3c2CC2CCC3O2)s1] | 0.04 (100.4) | 283 (113.9) |
| 39 | 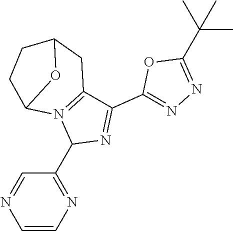 | 0.26 (100.1) | LA |
| 40 | 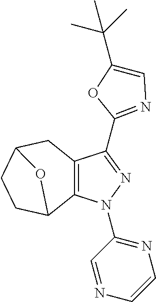 [SMILES: CC(C)(C)c1cnc(-c2nn(-c3cnccn3)c3c2CC2CCC3O2)o1] | 0.13 (99.7) | LA |
| 41 | 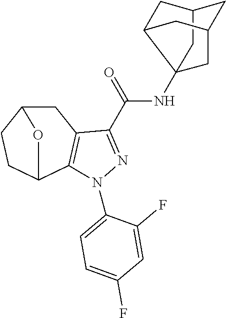 [SMILES: O=C(NC12CC3CC(CC1C3)C2)c1nn(-c2ccc(F)cc2F)c2c1CC1CCC2O1] | 0.08 (99.1) | 0.70 (98) |
| 42 | 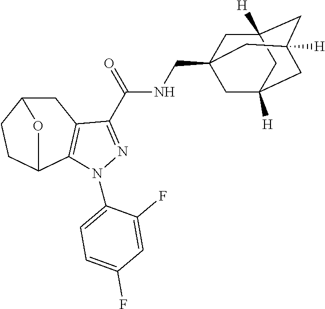 [SMILES: O=C(NC[C@]12C[C@@H]3C[C@@H](C[C@@H](C3)C1)C2)c1nn(-c2ccc(F)cc2F)c2c1CC1CCC2O1] | 10.3 (77.1) | 260 (98.2) |
| 44 | 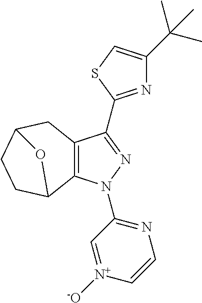 | 46.3 (84.6) | LA |
| 45 | 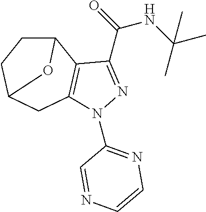 [SMILES: CC(C)(C)NC(=O)c1nn(-c2cnccn2)c2c1C1CCC(C2)O1] | 22.9(98.1) | LA |
| 46 | 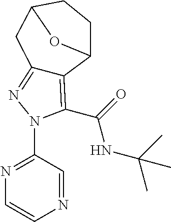 [SMILES: CC(C)(C)NC(=O)c1c2c(nn1-c1cnccn1)CC1CCC2O1] | 1650 (85.8) | LA |
| 47 | 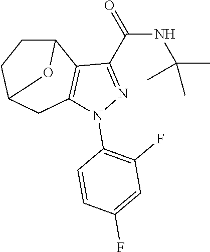 [SMILES: CC(C)(C)NC(=O)c1nn(-c2ccc(F)cc2F)c2c1C1CCC(C2)O1] | 559 (82.5) | LA |
| 48 | 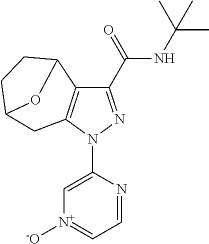 | 1.98 (99.9) | LA |
| 49 | 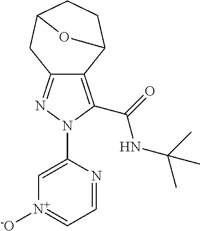 | 2673 (80.3) | LA |
| 50 | 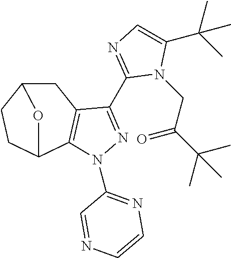 [SMILES: CC(C)(C)C(=O)Cn1c(C(C)(C)C)cnc1-c1nn(-c2cnccn2)c2c1CC1CCC2O1] | LA | LA |

### Formulation Example 1—Prophetic Example

### Solid, Oral Dosage Form

As a specific embodiment of an oral composition, 100 mg of the Cpd. 3, prepared as in Example 2 above, is formulated with sufficient finely divided lactose to provide a total amount of 580 to 590 mg to fill a size O hard gel capsule.

While the foregoing specification teaches the principles of the present invention, with examples provided for the purpose of illustration, it will be understood that the practice of the invention encompasses all of the usual variations, adaptations and/or modifications as come within the scope of the following claims and their equivalents.

Throughout this application, various publications may be cited. The disclosure of these publications is hereby incorporated by reference into this application to describe more fully the state of the art to which this invention pertains.

1. A compound of formula (I) 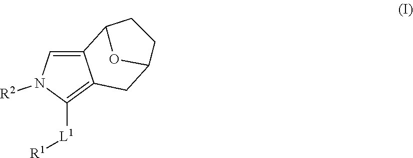
wherein
L¹-R¹ is selected from the group consisting of
—C(O)—NH—R¹, 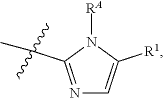
wherein R^{A} is hydrogen, methyl or ethyl; 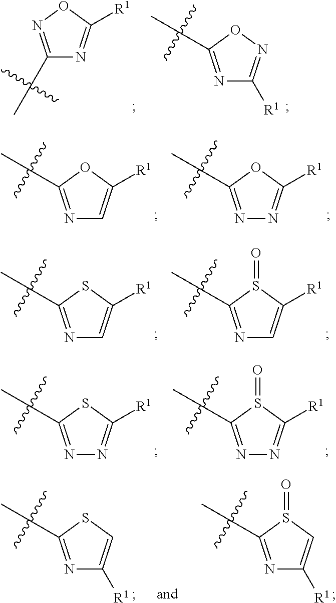
R¹ is selected from the group consisting of C₁₋₁₂alkyl, C₃₋₁₂cycloalkyl and -(C₁₋₂alkyl)-C₃₋₁₂-cycloalkyl;
wherein the C₁₋₁₂alkyl or C₃₋₁₂cycloalkyl, whether alone or as part of a substituent group may be optionally substituted with one to three substituents independently selected from the group consisting of halogen, hydroxy and fluorinated C₁₋₄alkyl;
provided that when -L¹-R¹ is other than —C(O)—NH—R¹, then R¹ is selected from the group consisting of C₁₋₁₂alkyl; wherein the C₁₋₁₂alkyl is optionally substituted with one to three substituents independently selected from the group consisting of halogen, hydroxy and fluorinated C₁₋₄alkyl;
R² is selected from the group consisting of phenyl, pyrazin-2-yl and pyrazin-2-yl-1-oxide;
wherein the phenyl is optionally substituted with one or more substituents independently selected from the group consisting of halogen, C₁₋₂alkyl and CF₃;
provided that when -L¹-R¹ is selected from the group consisting of 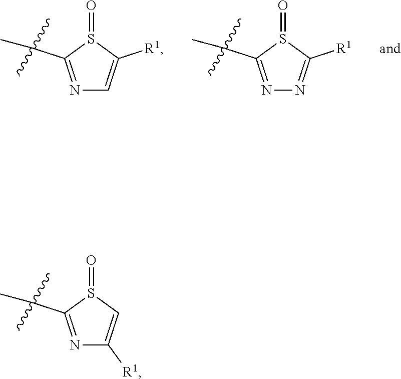
then R² is pyrazin-2-yl-1-oxide;
or a pharmaceutically acceptable salt thereof.

2. A compound as in claim 1, wherein
-L¹-R¹ is —C(O)—NH—R¹;
R¹ is selected from the group consisting of t-butyl, octahydro-2,5-methanopentalen-3-yl and adamant-1-yl-methyl-;
R² is selected from the group consisting of 2,4-difluoro-phenyl, pyrazin-2-yl and pyrazin-3-yl-1-oxide;
or a pharmaceutically acceptable salt thereof.

3. A compound of formula (II) 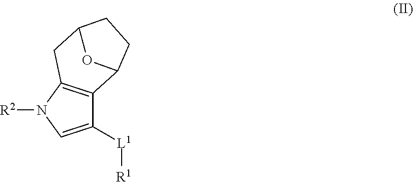
wherein
L¹-R¹ is selected from the group consisting of
—C(O)—NH—R¹, 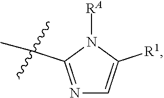
wherein R^{A} is hydrogen, methyl or ethyl; 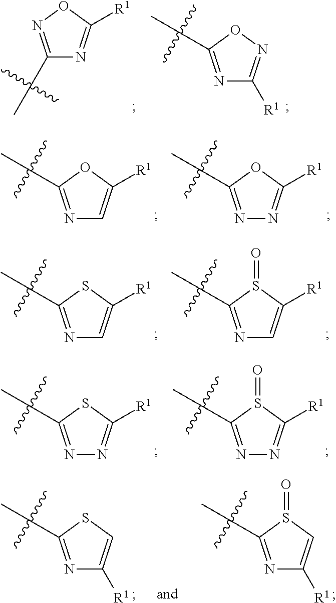
R¹ is selected from the group consisting of C₁₋₁₂alkyl, C₃₋₁₂cycloalkyl and -(C₁₋₂alkyl)-C₃-cycloalkyl;
wherein the C₁₋₁₂alkyl or C₃₋₁₂cycloalkyl, whether alone or as part of a substituent group may be optionally substituted with one to three substituents independently selected from the group consisting of halogen, hydroxy and fluorinated C₁₋₄alkyl;
provided that when -L¹-R¹ is other than —C(O)—NH—R¹, then R¹ is selected from the group consisting of C₁₋₁₂alkyl; wherein the C₁₋₁₂alkyl is optionally substituted with one to three substituents independently selected from the group consisting of halogen, hydroxy and fluorinated C₁₋₄alkyl;
R² is selected from the group consisting of phenyl, pyrazin-2-yl and pyrazin-2-yl-1-oxide;
wherein the phenyl is optionally substituted with one or more substituents independently selected from the group consisting of halogen, C₁₋₂alkyl and CF₃;
provided that when -L¹-R¹ is selected from the group consisting of 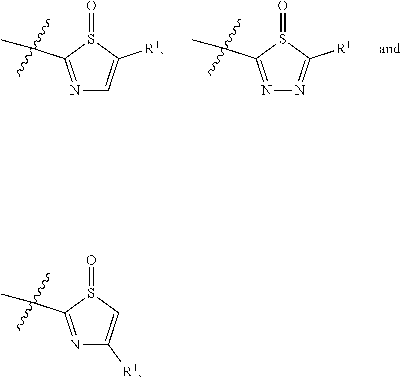
then R² is pyrazin-2-yl-1-oxide;
or a pharmaceutically acceptable salt thereof.

4. A compound as in claim 3, wherein
L¹-R¹ is selected from the group consisting of —C(O)—NH—R¹, 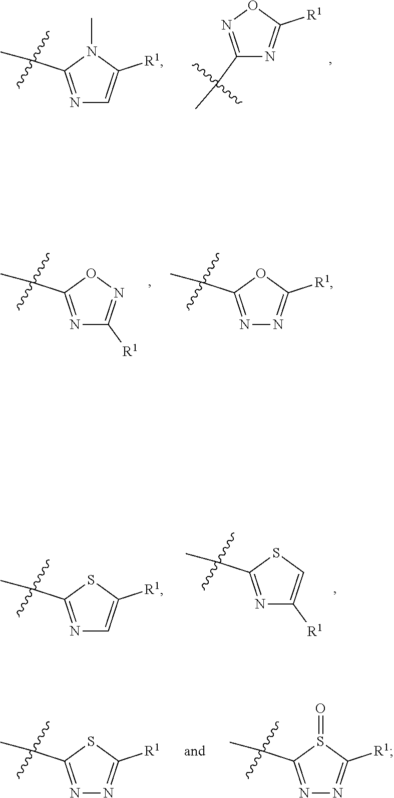
R¹ is selected from the group consisting of t-butyl, 2-methyl-n-but-2-yl, 1,1-dimethyl-2-hydroxy -ethyl, 1-hydroxy-3,3-dimethyl-n-but-2-yl, 1-(trifluoromethyl)-cycloprop-1-yl, 1-(trifluoromethyl)-cyclobut-1-yl, bicyclo[1.1.1]pent-1-yl, bicyclo[2.2.2]octan-1-yl, octahydro -2,5-methanopentalen-3-yl and (1R,3R,5S,7R)-tetracyclo[5.2.1.0^{3,8}.0^{5,8}]decan-3-yl-methyl;
R² is selected from the group consisting of 2,4-difluorophenyl, pyrazin-2-yl and pyrazin-3-yl-1-oxide;
provided that when -L¹-R¹ is 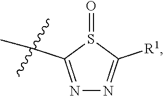
then R² is pyrazin-2-yl-1-oxide;
or a pharmaceutically acceptable salt thereof.

5. A compound as in claim 3, selected from the group consisting of N-(tert-butyl)-1-(pyrazin-2-yl)-1,4,5,6,7,8-hexahydro-5,8-epoxycyclohepta[c]pyrazole-3-carboxamide;
3-(3-(tert-butylcarbamoyl)-5,6,7,8-tetrahydro-5,8-epoxycyclohepta[c]pyrazol-1(4H)-yl)pyrazine 1-oxide;
N-(1-hydroxy-2-methylpropan-2-yl)-1-(pyrazin-2-yl)-1,4,5,6,7,8-hexahydro-5,8-epoxycyclohepta[c]pyrazole-3-carboxamide;
3-(3-((1-hydroxy-2-methylpropan-2-yl)carbamoyl)-5,6,7,8-tetrahydro-5,8-epoxycyclohepta[c]pyrazol-1(4H)-yl)pyrazine 1-oxide;
1-(pyrazin-2-yl)-N-(1-(trifluoromethyl)cyclopropyl)-1,4,5,6,7,8-hexahydro-5,8-epoxycyclohepta[c]pyrazole-3-carboxamide;
3-((5S,8R)-3-(((S)-1-hydroxy-3,3-dimethylbutan-2-yl)carbamoyl)-5,6,7,8-tetrahydro-5,8-epoxycyclohepta[c]pyrazol-1(4H)-yl)pyrazine 1-oxide;
3-((5R,8S)-3-(((S)-1-hydroxy-3,3-dimethylbutan-2-yl)carbamoyl)-5,6,7,8-tetrahydro-5,8-epoxycyclohepta[c]pyrazol-1(4H)-yl)pyrazine 1-oxide;
1-(pyrazin-2-yl)-N-(1-(trifluoromethyl)cyclobutyl)-1,4,5,6,7,8-hexahydro-5,8-epoxycyclohepta[c]pyrazole-3-carboxamide;
3-(3-((1-(trifluoromethyl)cyclopropyl)carbamoyl)-5,6,7,8-tetrahydro-5,8-epoxycyclohepta[c]pyrazol-1(4H)-yl)pyrazine 1-oxide;
and pharmaceutically acceptable salts thereof.

6. A compound as in claim 1, wherein
-L¹-R¹ is selected from the group consisting of —C(O)—NH—R¹, 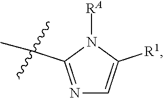
wherein R^{A} is selected from the group consisting of hydrogen, methyl and ethyl; 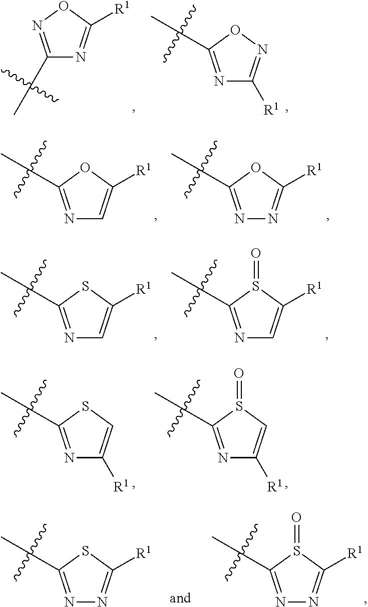
R¹ is selected from the group consisting of C₁₋₁₂alkyl, C₃₋₁₂cycloalkyl and -(C₁₋₂alkyl)-C₃₋₁₂cycloalkyl;
wherein the C₁₋₈alkyl or C₃₋₁₂cycloalkyl, whether alone or as part of a substituent group may be optionally substituted with one to two substituents independently selected from the group consisting of halogen, hydroxy and fluorinated C₁₋₂alkyl;
provided that when -L¹-R¹ is other than —C(O)—NH—R¹, then R¹ is C₁₋₈alkyl; wherein the C₁₋₁₂alkyl is optionally substituted with one to two substituents independently selected from the group consisting of halogen, hydroxy and fluorinated C₁₋₂alkyl;
R² is selected from the group consisting of phenyl, pyrazin-2-yl and pyrazin-2-yl-1-oxide;
wherein the phenyl is optionally substituted with one to two substituents independently selected from the group consisting of halogen, C₁₋₂alkyl and CF₃;
provided that when -L¹-R¹ is 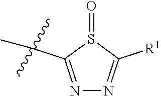
then R² is pyrazin-2-yl-1-oxide;
or a pharmaceutically acceptable salt thereof.

7. A compound as in claim 1, wherein
-L¹-R¹ is selected from the group consisting of —C(O)—NH—R¹, 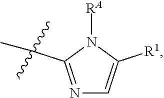
wherein R^{A} is selected from the group consisting of hydrogen, methyl and ethyl; 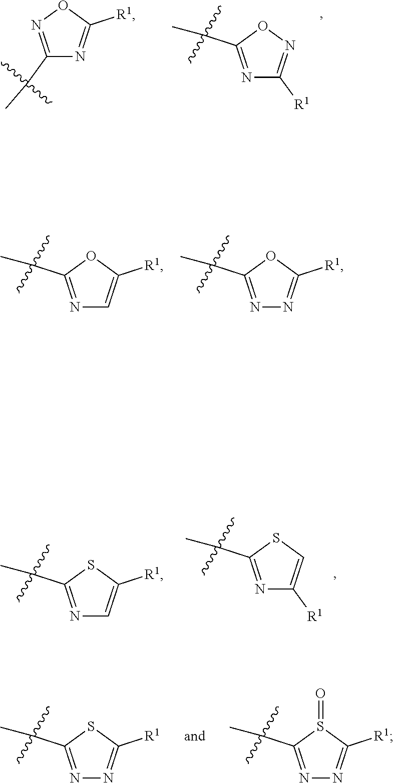
R¹ is selected from the group consisting of C₁₋₆alkyl, C₃₋₁₂cycloalkyl and —(CH₂)-C₃₋₁₂cycloalkyl;
wherein the C₁₋₆alkyl or C₃₋₁₂cycloalkyl, whether alone or as part of a substituent group may be optionally substituted a substituent selected from the group consisting of hydroxy and fluorinated C₁₋₂alkyl;
provided that when -L¹-R¹ is other than —C(O)—NH—R¹, then R¹ is selected from the group consisting of C₁₋₆alkyl;
R² is selected from the group consisting of phenyl, pyrazin-2-yl and pyrazin-2-yl-1-oxide;
wherein the phenyl is optionally substituted with one to two substituents independently selected from the group consisting of fluoro and CF₃;
provided that when -L¹-R¹ is 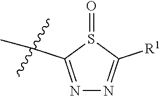
then R² is pyrazin-2-yl-1-oxide;
or a pharmaceutically acceptable salt thereof.

8. A compound as in claim 1, wherein
L¹-R¹ is selected from the group consisting of —C(O)—NH—R¹, 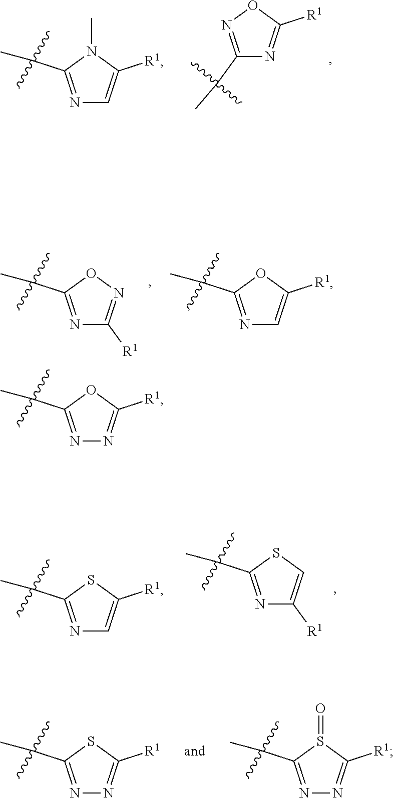
R¹ is selected from the group consisting of t-butyl, 2-methyl-n-but-2-yl, 1,1-dimethyl-2-hydroxy -ethyl, 1-hydroxy-3,3-dimethyl-n-but-2-yl, 1-(trifluoromethyl)-cycloprop-1-yl, 1-(trifluoromethyl)-cyclobut-1-yl, bicyclo[1.1.1]pent-1-yl, bicyclo[2.2.2]octan-1-yl, octahydro-2,5-methanopentalen-3-yl and (1R,3R,5S,7R)-tetracyclo[5.2.1.0^{3,8}.0^{5,8}]decan-3-yl-methyl and adamant-1-yl-methyl-;
provided that when -L¹-R¹ is other than —C(O)—NH—R¹, then R¹ is selected from the group consisting of t-butyl and 2-methyl-n-butry-2-yl;
R² is selected from the group consisting of 2,4-difluorophenyl, pyrazin-2-yl and pyrazin-3-yl-1-oxide;
provided that when -L¹-R¹ is 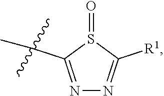
then R² is pyrazin-2-yl-1-oxide;
or a pharmaceutically acceptable salt thereof.

9. A compound selected from the group consisting 1-(5-(tert-butyl)-2-(1-(pyrazin-2-yl)-1,4,5,6,7,8-hexahydro-5,8-epoxycyclohepta[c]pyrazol-3-yl)-1H-imidazol-1-yl) -3,3-dimethylbutan-2-one and pharmaceutically acceptable salts thereof.

10. A pharmaceutical composition comprising a pharmaceutically acceptable carrier and a compound of Formula I, Formula II, or a combination thereof.

11-27. (canceled)

28. A method of treating a disorder, disease or condition modulated by the CB1, CB2 or dual CB1/CB2 receptor(s), selected from the group consisting of pain, neurodegenerative disorders, eating disorders, fibrotic diseases, or for weight loss, weight control or the treatment of obesity, comprising administering to a subject in need thereof a therapeutically effective amount of a compound of claim 1.

29. A method of treating a disorder, disease or condition modulated by the CB1, CB2 or dual CB1/CB2 receptor(s), selected from the group consisting of pain, neurodegenerative disorders, eating disorders, fibrotic diseases, or for weight loss, weight control or the treatment of obesity, comprising administering to a subject in need thereof a therapeutically effective amount of a compound of claim 3.

30. A method of treating a disorder, disease or condition modulated by the CB1, CB2 or dual CB1/CB2 receptor(s), selected from the group consisting of pain, neurodegenerative disorders, eating disorders, fibrotic diseases, or for weight loss, weight control or the treatment of obesity, comprising administering to a subject in need thereof a therapeutically effective amount of a composition of claim 10.
